(19) United States
(12) Patent Application Publication
(10) Pub. No.: US 2017/0088520 A1
(43) Pub. Date: Mar. 30, 2017

(54) DEUBIQUITINASE INHIBITORS

(71) Applicant: PharmAkea, Inc., San Diego, CA (US)

(72) Inventors: Andiliy LAI, San Diego, CA (US); John Howard HUTCHINSON, San Diego, CA (US); David LONERGAN, San Marcos, CA (US); Fei HUANG, La Mesa, CA (US)

(21) Appl. No.: 15/312,133

(22) PCT Filed: May 27, 2015
(86) PCT No.: PCT/US2015/032734

§ 371 Date: Nov. 17, 2016

Related U.S. Application Data
(60) Provisional application No. 62/006,767, filed on Jun. 2, 2014.

(51) Int. Cl. C07D 213/61; C07D 405/12; C07D 213/57; C07D 401/12; C07D 401/06; C07D 471/04; C07C 255/41; C07D 401/04

(52) U.S. Cl. CPC C07D 213/61; C07C 255/41; C07D 405/12; C07D 401/04; C07D 401/12; C07D 401/06; C07D 471/04; C07D 213/57; C07C 2101/08

(57) ABSTRACT

Described herein are compounds that are deubiquitinase inhibitors, methods of making pharmaceutical compositions and medicaments comprising such compounds, and methods of using such compounds in the treatment of conditions, diseases, or disorders that would benefit from inhibition of deubiquitinase activity.

### RELATED APPLICATIONS

This application claims the benefit of U.S. Provisional Patent Application No. 62/006,767 entitled “DEUBIQUITINASE INHIBITORS” filed on Jun. 2, 2014, which is incorporated herein by reference in its entirety.

### FIELD OF THE INVENTION

Described herein are compounds that are deubiquitinase inhibitors, methods of making pharmaceutical compositions and medicaments comprising such compounds, and methods of using such compounds in the treatment of conditions, diseases, or disorders that would benefit from inhibition of deubiquitinase activity.

### BACKGROUND OF THE INVENTION

Ubiquitination is a covalent posttranslational modification of cellular proteins involving a complex enzymatic cascade. Enzymes of the ubiquitination cascade are differentially expressed or activated in many diseases, including cancer. Protein ubiquitination is a dynamic two-way process that can be reversed or regulated by deubiquitinating enzymes (DUB). DUBs primarily serve to counterbalance ubiquitin-protein conjugation and also facilitate the cleavage of ubiquitin from its precursors and unanchored polyubiquitin chains. Thus, DUBs regulate and maintain the homeostasis of free ubiquitin pools in the cell. DUBs enhance protein stability by preventing protein degradation and dysregulation in the activity and expression of DUBs has been linked to cancer development and progression.

### SUMMARY OF THE INVENTION

In one aspect, described herein is a compound that has the structure of Formula (I), or a pharmaceutically acceptable salt, or solvate thereof:

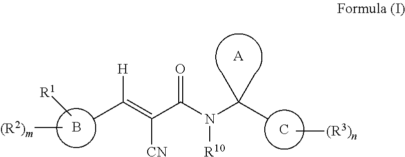

wherein,
ring A is a substituted or unsubstituted cycloalkylene, or substituted or unsubstituted heterocycloalkylene;
ring B is phenyl, naphthyl, or heteroaryl;
ring C is phenyl, naphthyl, or heteroaryl;
R¹ is selected from H, halogen, —OR⁴, —SR⁴, —N(R⁴)₂, —CN, substituted or unsubstituted alkyl, substituted or unsubstituted haloalkyl, substituted or unsubstituted phenyl, and substituted or unsubstituted heteroaryl;
each R² is independently selected from H, halogen, —OR⁴, —SR⁴, —N(R⁴)₂, —CN, —NO₂, substituted or unsubstituted alkyl, substituted or unsubstituted alkenyl, substituted or unsubstituted alkynyl, substituted or unsubstituted haloalkyl, —CO₂R⁴, C(═O)N(R⁴)₂, NHC(═O)R⁵, substituted or unsubstituted phenyl, and substituted or unsubstituted heteroaryl;
each R³ is independently selected from H, halogen, —CN, —NO₂, —OR⁴, —SR⁴, —S(═O)R⁵, —S(═O)₂R⁵, —S(═O)₂N(R⁴)₂, —NR⁴S(═O)₂R⁵, —C(═O)R⁵, —OC(═O)R⁵, —CO₂R⁴, —OCO₂R⁵, —N(R⁴)₂, —C(═O)N(R⁴)₂, —OC(═O)N(R⁴)₂, —NHC(═O)R⁵, —NHC(═O)OR⁵, unsubstituted or substituted alkyl, unsubstituted or substituted fluoroalkyl, unsubstituted or substituted alkenyl, unsubstituted or substituted alkynyl, unsubstituted or substituted heteroalkyl, unsubstituted or substituted cycloalkyl, unsubstituted or substituted aryl, unsubstituted or substituted heteroaryl, and -L³-L⁴-L⁵-L⁶-R⁹;
   L³ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁴ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, or —(OCH₂CH₂)ₛ—, s is 1, 2, 3, or 4;
   L⁵ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
   L⁶ is absent, unsubstituted or substituted alkylene, unsubstituted or substituted heteroalkylene;
   R⁹ is H, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, or unsubstituted or substituted heteroaryl;
each R⁴ is independently selected from H, C₁-C₆alkyl, C₁-C₆fluoroalkyl, C₁-C₆deuteroalkyl, C₃-C₆cycloalkyl, a substituted or unsubstituted phenyl, or a substituted or unsubstituted monocyclic heteroaryl; or two R⁴ groups attached to the same N atom are taken together with the N atom to which they are attached to form a substituted or unsubstituted heterocycle;
R⁵ is C₁-C₆alkyl, C₁-C₆fluoroalkyl, C₁-C₆deuteroalkyl, C₃-C₆cycloalkyl, a substituted or unsubstituted phenyl, a substituted or unsubstituted monocyclic heteroaryl, or a substituted or unsubstituted bicyclic heteroaryl;
R¹⁰ is H, C₁-C₆alkyl, or C₁-C₆haloalkyl;
m is 0, 1, 2, or 3;
n is 0, 1, 2 or 3.

For any and all of the embodiments, substituents are selected from among a subset of the listed alternatives. For example, in some embodiments, R¹⁰ is H, C₁-C₆alkyl, or C₁-C₆haloalkyl. In other embodiments, R¹⁰ is H or C₁-C₆alkyl. In other embodiments, R¹⁰ is H.

In some embodiments, ring A is a substituted or unsubstituted cycloalkylene.

In some embodiments, ring A is a substituted or unsubstituted monocyclic cycloalkylene, or substituted or unsubstituted bicyclic cycloalkylene.

In some embodiments, ring A is a substituted or unsubstituted monocyclic C₃-C₈cycloalkylene.

In some embodiments, ring A is a substituted or unsubstituted cyclopropylene, substituted or unsubstituted cyclobutylene, substituted or unsubstituted cyclopentylene, substituted or unsubstituted cyclohexylene, or substituted or unsubstituted cycloheptylene.

In some embodiments, the compound of Formula (I) has the structure of Formula (II), or a pharmaceutically acceptable salt or solvate thereof:

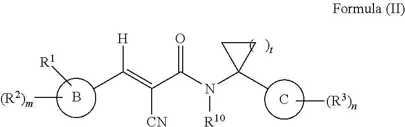

wherein,
t is 1, 2, 3, 4, or 5.

In some embodiments, ring A is a substituted or unsubstituted heterocycloalkylene.

In some embodiments, ring A is a substituted or unsubstituted monocyclic heterocycloalkylene, or substituted or unsubstituted bicyclic heterocycloalkylene.

In some embodiments, ring A is a substituted or unsubstituted monocyclic heterocycloalkylene.

In some embodiments, ring A is substituted or unsubstituted pyrrolidinyl, substituted or unsubstituted pyrrolidinonyl, substituted or unsubstituted tetrahydrofuranyl, substituted or unsubstituted tetrahydrofuranonyl, substituted or unsubstituted dihydrofuranonyl, substituted or unsubstituted dihydrofuranyl, substituted or unsubstituted tetrahydrothienyl, substituted or unsubstituted oxazolidinonyl, substituted or unsubstituted tetrahydropyranyl, substituted or unsubstituted dihydropyranyl, substituted or unsubstituted tetrahydrothiopyranyl, substituted or unsubstituted piperidinyl, substituted or unsubstituted morpholinyl, substituted or unsubstituted thiomorpholinyl, substituted or unsubstituted piperazinyl, substituted or unsubstituted aziridinyl, azetidinyl, substituted or unsubstituted oxetanyl, substituted or unsubstituted thietanyl, substituted or unsubstituted homopiperidinyl, substituted or unsubstituted oxepanyl, substituted or unsubstituted thiepanyl, substituted or unsubstituted oxazepinyl, substituted or unsubstituted diazepinyl, substituted or unsubstituted thiazepinyl, or substituted or unsubstituted 1,2,3,6-tetrahydropyridinyl.

In some embodiments, ring A is a substituted or unsubstituted bicyclic heterocycloalkylene.

In some embodiments, ring A is substituted or unsubstituted indolinyl, or substituted or unsubstituted indolinonyl.

In some embodiments, the compound of Formula (I) has the structure of Formula (III), or a pharmaceutically acceptable salt or solvate thereof:

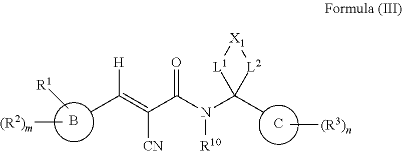

L¹ is —(C(R⁶)₂)ₚ—; p is 1, 2, 3, or 4;
L² is —(C(R⁶)₂)_{q}—; q is 0, 1, 2, 3, or 4;
X¹ is absent, —(C(R⁷)₂—, —O—, —S—, —S(O)—, —S(O)₂₋, —NR⁸—, —C(═O)—, —C(═O)NR⁴—, —C(═O)O—, —OC(═O)NR⁴—, or —NR⁴C(═O)NR⁴—;
   each R⁶ is independently selected from H, halogen, —OR⁴, substituted or unsubstituted alkyl, substituted or unsubstituted haloalkyl, substituted or unsubstituted phenyl, and substituted or unsubstituted heteroaryl;
   or two R⁶ on adjacent carbon atoms are taken together with the intervening atoms to form a monocyclic carbocycle or monocyclic heterocycle;
   each R⁷ is independently selected from H, halogen, —OR⁴, substituted or unsubstituted alkyl, substituted or unsubstituted haloalkyl, substituted or unsubstituted phenyl, and substituted or unsubstituted heteroaryl;
   or both R⁷ are taken together with the carbon atom to which they are attached to form a monocyclic carbocycle or monocyclic heterocycle;
   each R⁸ is independently selected from H, —S(═O)R⁵, —S(═O)₂R⁵, —S(═O)₂N(R⁴)₂, —C(═O)R⁵, —CO₂R⁵, or —C(═O)N(R⁴)₂.

In some embodiments, X¹ is absent, —(C(R⁷)₂—, —O—, —S—, —NR⁸—, or —C(═O)—.

In some embodiments, X¹ is absent.

In some embodiments, X¹ is —(C(R⁷)₂— or —C(═O)—.

In some embodiments, X¹ is —(C(R⁷)₂—; each R⁷ is independently selected from H, halogen, —O—C₁-C₆alkyl, —O—C₁-C₆haloalkyl, C₁-C₆alkyl, or C₁-C₆haloalkyl; or both R⁷ are taken together with the carbon atom to which they are attached to form a monocyclic carbocycle or monocyclic heterocycle.

In some embodiments, X¹ is —(C(R⁷)₂—; both R⁷ are taken together with the carbon atom to which they are attached to form a monocyclic carbocycle or monocyclic heterocycle.

In some embodiments, X¹ is —O— or —S.

In some embodiments, X¹ is —NR⁸—.

In some embodiments, each R⁸ is independently selected from H, —S(═O)₂R⁵, or —C(═O)R⁵.

In some embodiments, each R⁶ is independently selected from H, halogen, —O—C₁-C₆alkyl, —O—C₁-C₆haloalkyl, C₁-C₆alkyl, and C₁-C₆haloalkyl.

In some embodiments, each R⁶ is H.

In some embodiments, p is 1 or 2; and q is 1 or 2.

In some embodiments, ring B is phenyl.

In some embodiments, ring B is monocyclic heteroaryl.

In some embodiments, ring B is furanyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, or triazinyl.

In some embodiments, ring B is pyridinyl, pyrimidinyl, pyrazinyl, pyridazinyl, or triazinyl.

In some embodiments, ring is pyridinyl.

In some embodiments,

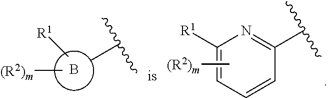

In some embodiments, ring B is furanyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, R¹ is selected from H, F, Cl, Br, —CN, substituted or unsubstituted alkyl, substituted or unsubstituted haloalkyl; each R² is independently selected from H, halogen, —OR⁴, —SR⁴, —N(R⁴)₂, —CN, —NO₂, substituted or unsubstituted alkyl, substituted or unsubstituted haloalkyl.

In some embodiments, R¹ is selected from H, F, Cl, or Br.

In some embodiments, ring C is phenyl, naphthyl, monocyclic heteroaryl or bicyclic heteroaryl.

In some embodiments, ring C is phenyl.

In some embodiments, ring C is monocyclic heteroaryl or bicyclic heteroaryl.

In some embodiments, ring C is monocyclic heteroaryl.

In some embodiments, ring C is furanyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, or triazinyl.

In some embodiments, ring C is furanyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, ring C is pyridinyl, pyrimidinyl, pyrazinyl, pyridazinyl, or triazinyl.

In some embodiments, ring C is bicyclic heteroaryl.

In some embodiments, ring C is quinolinyl, isoquinolinyl, quinazolinyl, quinoxalinyl, naphthyridinyl, indolyl, indazolyl, benzoxazolyl, benzisoxazolyl, benzofuranyl, benzothienyl, benzothiazolyl, benzimidazolyl, purinyl, cinnolinyl, phthalazinyl, pteridinyl, pyridopyrimidinyl, pyrazolopyrimidinyl, or azaindolyl.

In some embodiments, ring C is quinolinyl, isoquinolinyl, quinazolinyl, quinoxalinyl, indolyl, indazolyl, benzoxazolyl, benzisoxazolyl, benzofuranyl, benzothienyl, benzothiazolyl, or benzimidazolyl.

In some embodiments, each R³ is independently selected from H, halogen, —CN, —NO₂, —OR⁴, —SR⁴, —S(═O)R⁵, —S(═O)₂R⁵, —S(═O)₂N(R⁴)₂, —NR⁴S(═O)₂R⁵, —C(═O)R⁵, —OC(═O)R⁵, —CO₂R⁴, —OCO₂R⁵, —N(R⁴)₂, —C(═O)N(R⁴)₂, —OC(═O)N(R⁴)₂, —NHC(═O)R⁵, —NHC(═O)OR⁵, unsubstituted or substituted alkyl, unsubstituted or substituted fluroroalkyl, unsubstituted or substituted alkenyl, unsubstituted or substituted alkynyl, unsubstituted or substituted heteroalkyl, unsubstituted or substituted cycloalkyl, unsubstituted or substituted aryl, and unsubstituted or substituted heteroaryl.

In some embodiments, each R³ is independently selected from H, halogen, —CN, —NO₂, —OR⁴, —SR⁴, —S(═O)R⁵, —S(═O)₂R⁵, —S(═O)₂N(R⁴)₂, —NR⁴S(═O)₂R⁵, —C(═O)R⁵, —OC(═O)R⁵, —CO₂R⁴, —OCO₂R⁵, —N(R⁴)₂, —C(═O)N(R⁴)₂, —OC(═O)N(R⁴)₂, —NHC(═O)R⁵, —NHC(═O)OR⁵, C₁-C₆alkyl and C₁-C₆fluoroalkyl.

In some embodiments, each R³ is independently selected from H, halogen, —CN, —OR⁴, C₁-C₆alkyl and C₁-C₆fluroroalkyl.

In some embodiments, R³ is -L³-L⁴-L⁵-L⁶-R⁹; L and n is 1.

In some embodiments, -L³-L⁴-L⁵-L⁶- is C₁-C₆alkylene, —O—C₁-C₆alkylene, —NH—C₁-C₆alkylene, —(OCH₂CH₂)ᵣ—, —C₁-C₆alkylene-OC(═O)—C₁-C₆alkylene-, —C₁-C₆alkylene-NHC(═O)—C₁-C₆alkylene-, —C(═O)—C₁-C₆alkylene-C(═O)—, —(CH₂)ᵣ—OC(═O)—C₁-C₆alkylene-, —(CH₂)ᵣ—NHC(═O)—C₁-C₆alkylene-, —CH═N—NH—C(═O)—C₁-C₆alkylene-, or —C(CH₃)═N—NH—C(═O)—C₁-C₆alkylene-.

In some embodiments, R⁹ is unsubstituted or substituted monocyclic heterocycloalkyl or unsubstituted or substituted bicyclic heterocycloalkyl.

In some embodiments, R⁹ is unsubstituted or substituted aziridinyl, unsubstituted or substituted azetidinyl, unsubstituted or substituted pyrrolidinyl, unsubstituted or substituted pyrrolidinonyl, unsubstituted or substituted oxazolidinonyl, unsubstituted or substituted piperidinyl, unsubstituted or substituted morpholinyl, unsubstituted or substituted thiomorpholinyl, unsubstituted or substituted piperazinyl, unsubstituted or substituted maleimidyl, or unsubstituted or substituted biotinyl.

Any combination of the groups described above for the various variables is contemplated herein. Throughout the specification, groups and substituents thereof are chosen by one skilled in the field to provide stable moieties and compounds.

In one aspect, provided herein is a compound as described in Table 1 or Table 2 or Table 3, or a pharmaceutically acceptable salt or solvate thereof.

In another aspect, described herein is a pharmaceutical composition comprising a compound, or a pharmaceutically acceptable salt, or solvate thereof, as described herein, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is in the form of a tablet, a pill, a capsule, a liquid, a suspension, a gel, a dispersion, a solution, an emulsion, an ointment, or a lotion.

In one aspect, described herein is a method of treating a disease or condition in a mammal that would benefit from the inhibition of the activity of at least one deubiquitinating enzyme comprising administering to the mammal a compound, or pharmaceutically acceptable salt or solvate thereof, of Formula (I), or a pharmaceutically acceptable salt or solvate thereof. In some embodiments, the disease or condition is cancer, fibrosis, an autoimmune disease or condition, an inflammatory disease or condition, a neurodegenerative disease or condition or an infection.

In another aspect, described herein is a method of treating cancer in a mammal comprising administering to the mammal a compound of Formula (I), or a pharmaceutically acceptable salt or solvate thereof. In some embodiments, the cancer is a solid tumor. In some embodiments, the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, or skin cancer.

In any of the aforementioned aspects are further embodiments in which the effective amount of the compound of Formula (I), or a pharmaceutically acceptable salt thereof, is: (a) systemically administered to the mammal; and/or (b) administered orally to the mammal; and/or (c) intravenously administered to the mammal; and/or (d) administered by inhalation; and/or (e) t administered by nasal administration; or and/or (f) administered by injection to the mammal; and/or (g) administered topically to the mammal; and/or (h) administered by ophthalmic administration; and/or (i) administered rectally to the mammal; and/or (j) adminstered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the aforementioned aspects involving the treatment of diseases or conditions that would benefit from inhibition of the activity of at least one DUB enzyme are further embodiments comprising administering at least one additional agent in addition to the administration of a compound having the structure of Formula (I), or a pharmaceutically acceptable salt thereof. In various embodiments, each agent is administered in any order, including simultaneously.

In any of the embodiments disclosed herein, the mammal is a human.

In some embodiments, compounds provided herein are administered to a human.

In some embodiments, compounds provided herein are orally administered.

Articles of manufacture, which include packaging material, a compound of Formula (I), or a pharmaceutically acceptable salt thereof, within the packaging material, and a label that indicates that the compound or composition, or pharmaceutically acceptable salt, tautomers, pharmaceutically acceptable N-oxide, pharmaceutically active metabolite, pharmaceutically acceptable prodrug, or pharmaceutically acceptable solvate thereof, is used for inhibiting the activity of at least one DUB enzyme, or for the treatment, prevention or amelioration of one or more symptoms of a disease or condition that would benefit from inhibition of the activity of at least one DUB enzyme, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Ubiquitin is a 76 amino acid polypeptide that is covalently attached to proteins through the ubiquitination machinery. This machinery consists of an ubiquitin-activating enzyme (E1) that forms a thiol-ester intermediate with the C-terminal glycine residue of a ubiquitin monomer, a ubiquitin-conjugating enzyme (E2) to which the activated ubiquitin is transferred, and a ubiquitin ligase (E3) in which the final transfer of ubiquitin to a lysine residue of a protein substrate occurs. Ubiquitin addition can occur as a single molecule attachment to one or more lysine residues of the protein substrate (monoubiquitination) or as an ubiquitin chain (polyubiquitination). Ubiquitin contains seven internal lysine residues (K6, K11, K27, K29, K33, K48 and K63) and the ubiquitin chain can exist in several formats depending on the internal lysine residues utilized in the inter-ubiquitin linkage. These differently formatted ubiquitin chains further dictate the cellular fate of the ubiquitinated proteins. For example, ubiquitinated proteins containing the K48-linked chains are delivered to the 26S proteasome for degradation whereas ubiquitinated proteins containing the K63-linked chains alter target protein structure, localization and activity.

Ubiquitination is a reversible process and ubiquitin removal is tightly regulated by deubiquitinating enzymes. Deubiquitinating enzymes (DUBs) comprise a protease superfamily which mediates the removal of ubiquitin through a specific cleavage of isopeptide bonds at the C-terminus of ubiquitins. The human genome encodes ˜98 DUBs which are often part of large multi-protein complexes and are involved in several points along the ubiquitin pathway. For example, DUBs can process ubiquitins from polyubiquitin precursors, remove non-degradative ubiquitin signals, prevent protein degradation from the 26S proteasome and lysosomal pathways, maintain ubiquitin homeostasis and edit ubiquitin chains thereby modulating ubiquitin signals. DUB activity can enhance protein stability by preventing protein degradation and the dysregulation in the activity and expression of DUBs has been linked to multiple diseases.

DUB activities fall into three major functional categories. First, ubiquitin can be transcribed from several genes as a linear fusion of multiple ubiquitin molecules or with ribosomal proteins, such that the generation of free ubiquitin requires DUB activity. Second, DUBs can remove ubiquitin chains from post-translationally modified proteins, leading to reversal of ubiquitin signaling or to protein stabilization by rescue from either proteasomal (for example, cytosolic proteins) or lysosomal (for example, internalized receptors) degradation. However, once a commitment to these degradative machines has been made, associated DUB activities can recycle ubiquitin, thereby contributing to ubiquitin homeostasis. Third, DUBs can be used to edit the form of ubiquitin modification by trimming ubiquitin chains.

The process of ubiquitination-deubiquitination modulates a myriad of cellular processes including regulation of gene expression, cell-cycle progression, modifications of cell surface receptors, biogenesis of ribosomes, chromosomal segregation, protein degradation, DNA damage responses, apoptosis and signal transduction pathways. These cellular processes are particularly relevant in several disease states such as cancer, fibrosis, neurodegenerative disorders, autoimmune dysfunction and pathogenic infections. For example, the transforming growth factor beta (TGF-β) signaling pathway is a centralized command center for cell growth, cell differentiation, apoptosis and cellular homeostasis, and its disruption gives rise to neurodegenerative and fibrotic diseases, autoimmune dysfunction and cancer. Multiple deubiquitinases have been implicated in the regulation of the TGF-β pathway, including UCHL5 (also known as UCH37), USP4, USP9X, USP11 and USP15. Furthermore, mutations in several DUBs have been linked to diseases ranging from cancer to neurological disorders.

### Deubiquitinating Enzymes (DUBs)

DUBs are classified into five main families: ubiquitin-specific proteases (USPs), ubiquitin C-terminal hydrolases (UCHs), ovarian tumor proteases (OTUs), Machado-Joseph disease protein domain proteases (or Josephin domain) and JAB1/MPN/MOV34 metalloenzymes (JAMMs). The USP, UCH, OTU and Machado-Joseph disease protein domain proteases are characterized as cysteine proteases and the JAMM proteins are characterized as Zn²⁺-containing metalloproteases.

The USP family includes USP1, USP2, USP3, USP4, USP5, USP6, USP7, USP8, USP9X, USP9Y, USP10, USP11, USP12, USP13, USP14, USP15, USP16, USP17 (DUB3), USP18, USP19, USP20, USP21, USP22, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, USP47, USP48, USP49, USP50, USP51, USP52, USP53, USP54, CYLD, USPL1, and TL132. The UCH family includes UCHL1, UCHL3, UCHL5, and BAP1

The OUT family includes OTUB1, OTUB2, OTUD1, OTUD3, OTUD4, HIN1L, OTUD5, OTUD6A, OTUD6B, OTU1, A20, Cezanne-1, Cezanne-2, TRABID and VCPIP1.

The Machado-Joseph disease protein domain protease family includes ATXN3, ATXN3L, JOSD1 and JOSD2. The JAMM/MPN+ family includes BRCC36, CSN5, POH1 (also known as rpn11), AMSH, AMSH-LP, MPND, PSMD7, MYSM1, PRPF8, EIF3S3 and EIF3S5. Specific members of the DUB superfamily, for example USP and UCH family members such as USP9X, USP14, USP5, USP7, USP24, UCHL1 and UCHL5, have been linked to many diseases.

### USP9X

USP9X (or FAM) is associated with a variety of substrates such as SMAD4, MCL1, β-catenin, α-Synuclein, AF-6 and EFA6 which have been implicated in cancer and neurodegenerative diseases. For example, SMAD4 is a central transducer of TGFβ responses and interacts with various members of the R-SMADs forming heteromeric complexes which regulate gene expression. SMAD4 is the only coactivating SMAD (Co-SMAD) mediating signaling downstream of the TGFβ and BMP signaling pathways and its nuclear localization and activation are controlled by USP9x mediated deubiquitination (see Dupont et al, “FAM/USP9x, a deubiquitinating enzyme essential for TGF-β signaling, controls Smad4 monoubiquitination” Cell 136:123-135, 2009). Another critical protein for survival of stem and progenitor cells of multiple lineages is the pro-survival protein MCL1. It has been observed that down regulation or pharmacological inhibition of USP9x inhibits tumor cell growth by promoting degradation of MCL1 (see Schwickart et al., “Deubiquitinase USP9x stabilizes MCL1 and promotes tumour cell survival” Nature 463:103-107, 2010). In addition, USP9X has been observed to be upregulated in myeloma, breast, pancreatic, colon and prostate cancer cells.

The transcription factor E-twenty-six related gene (ERG) is overexpressed through gene fusion in ˜40% of prostate tumors and is a key driver of prostate cancer. USP9x deubiquitinates ERG leading to its accumulation. Pharmacological inhibition of USP9x results in the degradation of ERG and attenuates the growth of ERG positive prostate tumor cells in vitro and in vivo (see Wang et al., “Ablation of the oncogenic transcription factor ERG by deubiquitinase inhibition in prostate cancer” Proc Nat Acad Sci USA 111(11):4251-4256, 2014).

### USP24

USP24 is highly homologous to USP9x and in some cells has been localized in the nucleus where it plays a role in DNA repair by stabilizing DNA damage-binding protein 2 (Zhang et al., “The deubiquitinating protein USP24 interacts with DDB2 and regulates DDB2 stability” Cell Cycle 11:4378-4384, 2012). In addition USP24 gene transcription is tightly regulated by NFKβ, a protein which has significant roles in inflammation and apoptosis (see Wang et al., “Transcriptional regulation of human USP24 gene expression by NFKβ”. J Neurochem 128(6):818-828, 2014).

### USP7

USP7 (or HAUSP) is shown to interact with both viral and cellular proteins. In one aspect, USP7 interacts with PTEN, an antagonist in the AKT signaling pathway responsible for glucose metabolism, apoptosis, cell proliferation, transcription and cell migration. Reduced nuclear localization of PTEN is a common feature in aggressive cancers such as bladder, prostate, colon, liver, lung and prostate cancers. Indeed, overexpression of USP7 inhibits the nuclear localization of PTEN in prostate cancer cells (Song, et al., “The deubiquitinylation and localization of PTEN are regulated by a HAUSP-PML network,” Nature 455:813-817, 2008). In addition, inhibition of USP7 has been shown to induce apoptosis in multiple myeloma cells and to overcome cell resistance to chemotherapeutic agents such as bortezomib. Further, USP7 interacts with Vmw110, a viral protein that is required for the lytic cycle of herpes simplex virus, and with EBNA1, encoded by the Epstein-Barr virus, in which EBNA1 disrupts USP7's interaction with p53.

### USP14

USP14 is shown to be highly expressed in several cancer cell lines including multiple myeloma cells and colorectal carcinoma cells. Further, USP14 mediates the deubiquitination of Dishevelled (Dvl), a key regulator of the Wnt signaling pathway which shows abberant activation in diverse cancers including colorectal cancer (Jung, et al., “Deubiquitination of Dishevelled by Usp14 is required for Wnt signaling,” Oncogenesis, 2(8):e64, 2013). USP14 is one of three DUBs (the others are UCHL5 and POH1) in the 26S proteasome.

### USP5

USP5 (or isopeptidase T, ISOT) modulates the p53 pathway by preferentially processing unanchored polyubiquitin into free ubiquitins. The unanchored polyubiquitins compete with ubiquitinated p53 for proteasomal recognition and degradation to free ubiquitins. In one aspect, the inhibition of USP5 leads to the activation and stabilization of p53 by interfering with the degradation of ubiquitinated p53. (See, Dayal, et al, “Suppression of the deubiquitinating enzyme USP5 causes the accumulation of unanchored polyubiquitin and the activation of p53,” J. Biol. Chem. 284(8):5030-5041, 2009).

### UCHL1

UCHL1 (or PGP9.5) is upregulated in numerous cancers including lung, colorectal, pancreatic, thyroid, myeloma and B-lymphocyte cancers. For example, overexpression of UCHL1 is observed in 54% of all non-small-cell lung carcinomas and in 75% of stages II and III non-small-cell lung carcinomas. In colorectal cancer, 46% of the specimens expressed UCHL1. In addition, overexpression of UCHL1 has been correlated to increased tumor size and invasiveness in lung cancer patients and has been negatively correlated to postoperative survival in pancreatic cancer patients. Further, UCHL1 is shown to interact and colocalize with JAB1, a Jun activation domain-binding protein that interacts with p27. This interaction and colocalization may contribute to the degradation of p27, a cyclin-dependent kinase inhibitor whose loss in epithelial cancers has been correlated with pathological tumor grade where high-grade tumors exhibit significantly lower expression of p27 than their counterparts. The decreased expression of p27 has also been observed in breast, lung, PTEN prostate, colon, skin and ovarian cancers and has been correlated with cancer development and poor survival.

### UCHL5 (Also Known as UCH37)

UCHL5 interacts with glucose-regulated protein 78 which is essential for cell viability and can serve as a predictor of recurrence of hepatocellular carcinoma (see, Hirohashi, et al., “p78/MCRS1 forms a complex with centrosomal protein Nde1 and is essential for cell viability,” Oncogene 25:425-479, 2006). In some cases, UCHL5 can further serve as a predictor for overall survival (OS) and disease-free survival (DFS) in esophageal squamous cell carcinoma (ESCC) patients (see, Chen, et al., “Expression and clinical significance of UCH37 in human esophageal squamous cell carcinoma,” Dig Dis Sci 57:2310-2017, 2012). USHL5 is one of the three DUBs (the others are USP14 and POH1) in the 26S proteasome.

### DUBs and DUB Inhibition

Disclosed herein are compositions and methods of inhibiting a deubiquitinating enzyme (DUB). In some embodiments, a compound described herein inhibits all members of the DUB superfamily. In some embodiments, a compound described herein inhibits one or more members of the DUB superfamily. In some embodiments, the DUBs include USP1, USP2, USP3, USP4, USP5, USP6, USP7, USP8, USP9X, USP9Y, USP10, USP11, USP12, USP13, USP14, USP15, USP16, USP17 (DUB3), USP18, USP19, USP20, USP21, USP22, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, USP47, USP48, USP49, USP50, USP51, USP52, USP53, USP54, CYLD, USPL1, TL132, UCHL1, UCHL3, UCHL5, BAP1, UCH37, OTUB1, OTUB2, OTUD1, OTUD3, OTUD4, HIN1L, OTUD5, OTUD6A, OTUD6B, OTU1, A20, Cezanne1, Cezanne 2, TRABID, VCPIP1, ATXN3, ATXN3L, JOSD1, JOSD2, BRCC36, CSN5, POH1, AMSH, AMSH-LP, MPND, MYSM1, PRPF8, PSMD7, EIF3S3 and EIF3S5

In some embodiments, a compound described herein inhibits, USP1, USP2, USP3, USP4, USP5, USP6, USP7, USP8, USP9X, USP9Y, USP10, USP11, USP12, USP13, USP14, USP15, USP16, USP17 (DUB3), USP18, USP19, USP20, USP21, USP22, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, USP47, USP48, USP49, USP50, USP51, USP52, USP53, USP54, CYLD, USPL1, TL132, UCHL1, UCHL3, UCHL5, BAP1, UCH37, OTUB1, OTUB2, OTUD1, OTUD3, OTUD4, HIN1L, OTUD5, OTUD6A, OTUD6B, OTU1, A20, Cezanne1, Cezanne 2, TRABID, VCPIP1, ATXN3, ATXN3L, JOSD1, JOSD2, BRCC36, CSN5, POH1 (also known as rpn11), AMSH, AMSH-LP, MPND, MYSM1, PRPF8, PSMD7, EIF3S3 and EIF3S5 or any combinations thereof.

In some embodiments, a compound described herein inhibits USP9X, USP14, USP5, USP24, or UCHL5, or any combinations thereof

In some embodiments, the compound described herein inhibits a portion of a DUB. In some embodiments, the portion of the DUB comprises one or more domains of the DUB. In some embodiments, the compound described herein inhibits a portion of a DUB that is a conserved portion among the members of the DUB. In some embodiments, the compound described herein inhibits a portion of a DUB that is not conserved among the members of the DUB. In some embodiments, the compound described herein inhibits a portion that is conserved among USP9X, USP14, USP5, USP24, UCHL3 and UCHL5.

### DUBs and Diseases

Disclosed herein is a method of treating a disease or condition that would benefit from inhibition of the activity of one or more DUBs comprising administering a compound described herein, or a pharmaceutically acceptable salt thereof, to a mammal in need thereof. In some embodiments, disclosed herein is a method of reducing the progression of a disease associated with DUB activity, reversing the progression of a disease associated with DUB activity, extending the period of remission of a disease associated with DUB activity, or eliminating a disease associated with DUB activity in a mammal in need thereof comprising administering to the mammal a compound described herein. In some embodiments, the method of treatment refers to stopping the progression of a disease, partial or complete elimination of a disease, reversing progression of a disease, stopping, reducing or reversing episodes of worsening or relapses of a disease, or prolonging episodes of remission of a disease in a subject. In some embodiments, the disease is selected from a cancer, a fibrosis, an autoimmune disease or condition, a neurodegenerative disease or condition, or an infection.

### Cancer

In some embodiments, described herein is a method of treating cancer in a mammal comprising inhibiting the activity of one or more deubiquitinase enzymes in the mammal. In some embodiments, the method comprises administering a compound described herein, or a pharmaceutically acceptable salt thereof, to the mammal with a cancer. In some embodiments, the cancer is a solid tumor. In some embodiments, the solid tumor is a sarcoma or carcinoma. In some embodiments, the solid tumor is a sarcoma. In some embodiments, the solid tumor is a carcinoma. In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extraskeletal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. In some embodiments, the carcinoma is selected from anal cancer; appendix cancer; AIDS-related cancer; bile duct cancer (e.g. cholangiocarcinoma); bladder cancer; bone tumor; brain tumor; breast cancer; cervical cancer; colon cancer; cancer of Unknown Primary (CUP); esophageal cancer; eye cancer; fallopian tube cancer; gastroenterological cancer; head and neck cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the tumor is a metastatic tumor. In some embodiments, the cancer is a metastatic cancer.

In some embodiments, the cancer is selected from breast, bone, brain, colorectal, kidney, prostate and skin cancers. In some embodiments, the cancer is breast cancer. In some embodiments, the cancer is bone cancer. In some embodiments, the cancer is brain cancer. In some embodiments, the cancer is colorectal cancer. In some embodiments, the cancer is kidney cancer. In some embodiments, the cancer is prostate cancer. In some embodiments, the cancer is skin cancer.

In some embodiments, the breast cancer is ductal carcinoma in situ (intraductal carcinoma), lobular carcinoma in situ, invasive (or infiltrating) ductal carcinoma, invasive (or infiltrating) lobular carcinoma, inflammatory breast cancer, triple-negative breast cancer, paget disease of the nipple, phyllodes tumor, angiosarcoma or invasive breast carcinoma. In some embodiments, the invasive breast carcinoma is further categorized into subtypes. In some embodiments, the subtypes include adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, micropapillary carcinoma or mixed carcinoma.

In some embodiments, the bone cancer is chordrosarcoma, fibrosarcoma, lymphoma, malignant fibrous histiocytoma, metastatic bone disease, myeloma, osteosarcoma (OS) or Ewing's sarcoma. In some embodiments, chondrosarcoma is further classified as chondrosarcoma not otherwise specified, juxtacortical chondrosarcoma, myxoid chondrosarcoma, mesenchymal chondrosarcoma, clear cell chondrosarcoma and dedifferentiated chondrosarcoma. In some embodiments, osteosarcomas is further classified as conventional osteosarcomas (e.g. osteoblastic, chondroblastic, fibroblastic, epithelioid, giant-cell rich, small cell or telangiectatic), cortex-associated osteosarcomas (e.g. parosteal, dedifferentiated parosteal, periosteal, high-grade surface, or intracortical), low-grade (central) osteosarcomas, osteoblastoma-like osteosarcomas, disease-associated osteosarcoma (e.g. osteosarcoma in Paget's disease, osteosarcoma in fibrous dysplasia, or osteosarcomas in Mazabraud's disease), multicentric osteosarcomas, post-irradiation osteosarcoma, or osteosarcoma of the gnathic bones.

In some embodiments, the brain tumor is adenoma, astrocytoma, atypical teratoid rhaboid tumor (ATRT), chondrosarcoma, chordomas, choroid plexus, craniopharyngioma, cysts, ependymoma, germ cell tumor, glioblastoma, glioma, hemangioma, lipoma, medulloblastoma, meningioma, metastatic brain tumor, neurofibroma, neuronal and mixed neuronal-glial tumors, oligoastrocytoma, oligodendroglioma, pineal tumors, pituitary tumors, PNET or schwannoma.

In some embodiments, the colorectal cancer is adenocarcinoma (e.g. mucinous adenocarcinoma, signet ring cell adenocarcinoma), gastrointestinal carcinoid tumor, gastrointestinal stromal tumor, primary colorectal lymphoma, leiomyosarcoma, melanoma or squamous cell carcinoma.

In some embodiments, the kidney cancer is renal cell carcinoma, renal pelvis carcinoma, squamous cell carcinoma, juxtaglomerular cell tumor, angiomyolipoma, renal oncocytoma, Bellini duct carcinoma, clear cell sarcoma of the kidney, mesoblastic nephroma, Wilms' tumor, mixed epithelial stromal tumor, clear cell adenocarcinoma, transitional cell carcinoma (urothelial cell carcinoma), inverted papilloma, renal lymphoma, teratoma, carcinosarcoma, carcinoid tumor off the renal pelvis or oncocytomas. In some embodiments, renal cell carcinoma is further categorized into chromophobe renal cell carcinoma (CCRCC), papillary renal cell carcinoma (PRCC), clear cell renal cell carcinoma (CCRCC), multilocular cystic renal cell carcinoma, carcinoma of the collecting ducts of Bellini, medullary carcinoma, Xp11.2 translocation carcinoma, mucinous tubular spindle cell carcinoma and post-neuroblastoma renal cell carcinoma.

In some embodiments, the prostate cancer is acinar adenocarcinoma, ductal adenocarcinoma, transitional cell (or urothelial) cancer, squamous cell cancer, carcinoid, small cell cancer, sarcomas or sarcomatoid cancers.

In some embodiments, the skin cancer is basal cell carcinoma, squamous cell carcinoma or malignant melanoma. In some embodiments, the basal cell carcinoma is further classified as nodular and nodular-ulcerative basal cell carcinoma, pigmented basal cell carcinoma, superficial basal cell carcinoma and morphoeic basal cell carcinoma. In some embodiments, the squamous cell carcinoma is further classified as Bowen's disease, papillary thyroid carcinoma, verrucous squamous cell carcinoma, papillary squamous cell carcinoma, squamous cell carcinoma, large cell keratinizing squamous cell carcinoma, large cell nonkeratinizing squamous cell carcinoma, small cell keratinizing squamous cell carcinoma, spindle cell squamous cell carcinoma, adenoid/pseudoglandular squamous cell carcinoma, intraepidermal squamous cell carcinoma, lymphoepithelial carcinoma, keratoacanthoma, Erythroplasia of Queyrat and Marjolin's ulcer. In some embodiments, the malignant melanoma is further classified as lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, melanoma with small nevus-like cells, melanoma with features of a spitz nevus and uveal melanoma.

In some embodiments, the cancer is a hematologic cancer. In some embodiments, the hematologic cancer is a leukemia, a lymphoma, a myeloma, a non-Hodgkin's lymphoma, a Hodgkin's lymphoma, or a B-cell malignancy. In some embodiments, the hematologic cancer is selected from acute lymphoblastic leukemia (ALL), acute myelogenous leukemia (AML), chronic lymphocytic leukemia (CLL), chronic myelogenous leukemia (CML), hairy cell leukemia (HCL), T-cell prolymphocytic leukemia (T-PLL), large granular lymphocytic leukemia, adult T-cell leukemia, aggressive NK cell leukemia, small lymphocytic lymphoma (SLL), high risk CLL, non-CLL/SLL lymphoma, follicular lymphoma (FL), diffuse large B-cell lymphoma (DLBCL), activated B-cell diffuse large B-cell lymphoma (ABC-DLBCL), germinal center diffuse large B-cell lymphoma (GCB DLBCL), double-hit diffuse large B-cell lymphoma (DH-DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, lymphomatoid granulomatosis, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hepatosplenic gamma-delta T-cell lymphoma, lymphoblastic lymphoma, nasal NK/T-cell lymphomas, Sezary syndrome, AIDS-related lymphoma, treatment-related T-cell lymphomas, classical non-Hodgkin lymphoma (e.g. nodular sclerosing, mixed cellularity, lymphocyte rich and lymphocyte depleted) and nodular lymphocyte predominant type.

### Fibrosis

In some embodiments, described herein is a method of treating fibrosis in a mammal comprising inhibiting the activity of one or more deubiquitinase enzymes in the mammal. In some embodiments, the method comprises administering a compound described herein, or a pharmaceutically acceptable salt thereof, to the mammal with a fibrosis. In some embodiments, disclosed herein are methods of treating fibrosis with a compound disclosed herein.

“Fibrosis,” as used herein, refers to the accumulation of extracellular matrix constituents that occurs following trauma, inflammation, tissue repair, immunological reactions, cellular hyperplasia, and neoplasia.

In some embodiments, disclosed herein is a method of reducing fibrosis in a tissue comprising contacting a fibrotic cell or tissue with a compound disclosed herein, in an amount sufficient to decrease or inhibit the fibrosis. In some embodiments, the fibrosis includes a fibrotic condition.

In some embodiments, reducing fibrosis, or treatment of a fibrotic condition, includes reducing or inhibiting one or more of: formation or deposition of extracellular matrix proteins; the number of pro-fibrotic cell types (e.g., fibroblast or immune cell numbers); cellular collagen or hydroxyproline content within a fibrotic lesion; expression or activity of a fibrogenic protein; or reducing fibrosis associated with an inflammatory response.

In some embodiments, the fibrotic condition is primary fibrosis. In some embodiments, the fibrotic condition is idiopathic. In some embodiments, the fibrotic condition is associated with (e.g., is secondary to) a disease; a toxin; an insult (e.g., an environmental hazard); a medical treatment, or a combination thereof.

In some embodiments, the fibrotic condition is a fibrotic condition of the lung (pulmonary fibrosis), a fibrotic condition of the liver (renal fibrosis), a fibrotic condition of the heart or vasculature (cardiac fibrosis), a fibrotic condition of the kidney (renal fibrosis), a fibrotic condition of the skin, a fibrotic condition of the gastrointestinal tract, or a combination thereof.

In some embodiments, the fibrotic condition is a fibrotic condition of the lung. In some embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), bronchiolitis obliterans, or bronchiectasis. In some embodiments, the fibrotic condition of the lung treated with the methods of the invention is associated with (e.g., secondary to) a cancer treatment.

In some embodiments, the fibrotic condition is a fibrotic condition of the liver.

In some embodiments, the fibrotic condition is a fibrotic condition of the lung.

In some embodiments, the fibrotic condition is a fibrotic condition of the heart.

In some embodiments, the fibrotic condition is a fibrotic condition of the kidney.

In some embodiments, the fibrotic condition is a fibrotic condition of the skin.

In some embodiments, the fibrotic condition is a fibrotic condition of the gastrointestinal tract.

In some embodiments, the fibrotic condition is a fibrotic condition of the peritoneum.

In some embodiments, the fibrotic condition is a fibrotic condition of the bone marrow.

In some embodiments, the fibrotic condition is a fibrotic condition of the eye.

### Additional Diseases

In some embodiments, a deubiquitinase inhibitor is used in the treatment of an autoimmune disease or condition. In some embodiments, the autoimmune disease or condition is lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjögren's syndrome, multiple sclerosis, Guillain-Barré syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, Kawasaki's Disease, idiopathic thrombocytopenic purpura, multiple sclerosis, optic neuritis, uveitis, scleroderma, primary biliary cirrhosis, Reiter's syndrome, Takayasu's arteritis, temporal arteritis, optic neuritis, warm autoimmune hemolytic anemia, Wegener's granulomatosis, psoriasis, diabetes mellitus (Type I), alopecia universalis, Behçet's disease, chronic fatigue syndrome, coeliac disease, Crohn's disease, ulcerative colitis, dysautonomia, endometriosis, interstitial cystitis, neuromyotonia, scleroderma, or vulvodynia.

In some embodiments, a deubiquitinase inhibitor is used in the treatment of an inflammatory disease or condition. In some embodiments, the inflammatory disease or condition is asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In some embodiments, described herein is a method of treating a neurodegenerative disease or condition in a mammal comprising inhibiting the activity of one or more deubiquitinase enzymes in the mammal. In some embodiments, the method comprises administering a compound described herein, or a pharmaceutically acceptable salt thereof, to the mammal with a neurodegenerative disease.

In some embodiments, the neurodegenerative disease is selected from Alzheimer's disease, Parkinson's disease, Huntington disease, amyotrophic lateral sclerosis (ALS), spinocerebellar ataxias and Charcot-Marie-Tooth disease.

In some embodiments, described herein is a method of treating an infection in a mammal comprising inhibiting the activity of one or more deubiquitinase enzymes in the mammal. In some embodiments, the method comprises administering a compound described herein, or a pharmaceutically acceptable salt thereof, to the mammal with an infection.

In some embodiments, the infection is caused a bacterium, a virus, a fungus or a parasite. In some embodiments, the infection is caused by a virus. Examplary virus include, but are not limited to, cytomegalovirus, encephalomyocarditis virus (EMCV), Epstein-Barr virus, hepatitis B, hepatitis C, Kaposi's sarcoma herpesvirus, human immunodeficiency virus, human papillomavirus, human T-cell leukemia virus-1influenza virus, Merkel cell polyomavirus and respiratory syncytial virus.

In some embodiments, the infection is caused by a bacterium. Examplary bacteria include, but are not limited to, Burkholderia cenocepacia, Chlamydia pneumoniae, Escherichia coli, Helicobacter pylori, Listeria monocytogenes, Mycobacterium tuberculosis, Pseudomonas aeruginosa, Salmonella typhi, Salmonella enteric, Shigella flexneri, Stenotrophomonas maltopilia, Sthaphylococcus aureus and methicillin-resistant Staphylococcus Aureus (MRSA).

In some embodiments, the infection is caused by a fungus. Examplary fungi include, but are not limited to, Aspergillus fumigates, Aspergillus terreus, Peniciliun sp. Chrysonilia sp, Candida albicans, Candida glabrata and Exophiala dermatitidis.

In some embodiments, the infection is caused by a parasite. Examplary parasites include, but are not limited to, Clonorchis sinensis, liver flukes, Opisthorchis viverrini, Schistosoma haematobium and Schistosoma japonicum.

### Compounds

Compounds described herein, including pharmaceutically acceptable salts, prodrugs, active metabolites and pharmaceutically acceptable solvates thereof, are DUB inhibitors. In some embodiments, compounds described herein inhibit the activity of at least one deubiquitinase enzyme. In some embodiments, at least one deubiquitinase enzyme is USP9x, USP5, USP14, USP24 and/or UCHL5. In some embodiments, compounds described herein inhibit the activity of one or more kinases. In some embodiments, the one or more kinases include non-receptor tyrosine kinases. In some embodiments, the one or more kinases include one or more Janus kinases. In some embodiments, the one or more kinases include Jak/Stat kinases. In some embodiments, the one or more kinases include Jak/Stat, and/or Jak2/Stat3. In some embodiments, compounds described herein are used to treat any one of the diseases or conditions described herein by inhibiting the activity of one or more kinases. In some embodiments, compounds described herein are used to treat cancer in a mammal by inhibiting the activity of one or more kinases.

In one aspect, described herein is a compound that has the structure of Formula (I), or a pharmaceutically acceptable salt, or solvate thereof:

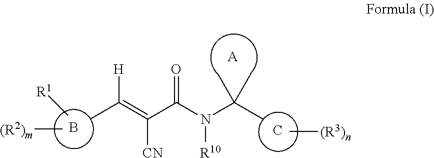

wherein,
ring A is a substituted or unsubstituted cycloalkylene, or substituted or unsubstituted heterocycloalkylene;
ring B is phenyl, naphthyl, or heteroaryl;
ring C is phenyl, naphthyl, or heteroaryl;
R¹ is selected from H, halogen, —OR⁴, —SR⁴, —N(R⁴)₂, —CN, substituted or unsubstituted alkyl, substituted or unsubstituted haloalkyl, substituted or unsubstituted phenyl, and substituted or unsubstituted heteroaryl;
each R² is independently selected from H, halogen, —OR⁴, —SR⁴, —N(R⁴)₂, —CN, —NO₂, substituted or unsubstituted alkyl, substituted or unsubstituted haloalkyl, —CO₂R⁴, C(═O)N(R⁴)₂, NHC(═O)R⁵, substituted or unsubstituted phenyl, and substituted or unsubstituted heteroaryl;
each R³ is independently selected from H, halogen, —CN, —NO₂, —OR⁴, —SR⁴, —S(═O)R⁵, —S(═O)₂R⁵, —S(═O)₂N(R⁴)₂, —NR⁴S(═O)₂R⁵, —C(═O)R⁵, —OC(═O)R⁵, —CO₂R⁴, —OCO₂R⁵, —N(R⁴)₂, —C(═O)N(R⁴)₂, —OC(═O)N(R⁴)₂, —NHC(═O)R⁵, —NHC(═O)OR⁵, unsubstituted or substituted alkyl, unsubstituted or substituted fluoroalkyl, unsubstituted or substituted alkenyl, unsubstituted or substituted alkynyl, unsubstituted or substituted heteroalkyl, unsubstituted or substituted cycloalkyl, unsubstituted or substituted aryl, unsubstituted or substituted heteroaryl, and -L³-L⁴-L⁵-L⁶-R⁹;
   L³ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁴ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, or —(OCH₂CH₂)ₛ—, s is 1, 2, 3, or 4;
   L⁵ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—;
   L⁶ is absent, unsubstituted or substituted alkylene, unsubstituted or substituted heteroalkylene;
   R⁹ is H, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, or unsubstituted or substituted heteroaryl;
each R⁴ is independently selected from H, C₁-C₆alkyl, C₁-C₆fluoroalkyl, C₁-C₆deuteroalkyl, C₃-C₆cycloalkyl, a substituted or unsubstituted phenyl, or a substituted or unsubstituted monocyclic heteroaryl; or two R⁴ groups attached to the same N atom are taken together with the N atom to which they are attached to form a substituted or unsubstituted heterocycle;
R⁵ is C₁-C₆alkyl, C₁-C₆fluoroalkyl, C₁-C₆deuteroalkyl, C₃-C₆cycloalkyl, a substituted or unsubstituted phenyl, a substituted or unsubstituted monocyclic heteroaryl, or a substituted or unsubstituted bicyclic heteroaryl;
R¹⁰ is H, C₁-C₆alkyl, or C₁-C₆haloalkyl;
m is 0, 1, 2, or 3;
n is 0, 1, 2 or 3.

For any and all of the embodiments, substituents are selected from among a subset of the listed alternatives. For example, in some embodiments, R¹⁰ is H, C₁-C₆alkyl, or C₁-C₆haloalkyl. In other embodiments, R¹⁰ is H or C₁-C₆alkyl. In other embodiments, R¹⁰ is H.

In some embodiments, m is 0, 1, 2, or 3. In some embodiments, m is 0, 1 or 2. In some embodiments, m is 0 or 1.

In some embodiments, n is 0, 1, 2, or 3. In some embodiments, n is 0, 1 or 2. In some embodiments, n is 0 or 1.

In some embodiments, ring A is a substituted or unsubstituted cycloalkylene. In some embodiments, ring A is a substituted or unsubstituted monocyclic cycloalkylene, or substituted or unsubstituted bicyclic cycloalkylene. In some embodiments, ring A is a substituted or unsubstituted monocyclic C₃-C₈cycloalkylene. In some embodiments, ring A is a substituted or unsubstituted monocyclic C₃-C₆cycloalkylene. In some embodiments, ring A is a substituted or unsubstituted cyclopropylene, substituted or unsubstituted cyclobutylene, substituted or unsubstituted cyclopentylene, substituted or unsubstituted cyclohexylene, or substituted or unsubstituted cycloheptylene. In some embodiments, ring A is a substituted or unsubstituted cyclopropylene, substituted or unsubstituted cyclobutylene, substituted or unsubstituted cyclopentylene, or substituted or unsubstituted cyclohexylene. In some embodiments, ring A is a substituted or unsubstituted cyclobutylene, substituted or unsubstituted cyclopentylene, or substituted or unsubstituted cyclohexylene. In some embodiments, ring A is a substituted or unsubstituted cyclopentylene.

In some embodiments, the compound of Formula (I) has the structure of Formula (II), or a pharmaceutically acceptable salt or solvate thereof:

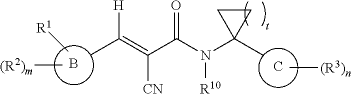

wherein,
t is 1, 2, 3, 4, or 5.

In some embodiments, t is 1, 2, 3, 4, or 5. In some embodiments, t is 1, 2, 3 or 4. In some embodiments, t is 1, 2 or 3. In some embodiments, t is 2, 3, 4, or 5. In some embodiments, t is 3, 4, or 5. In some embodiments, t is 1. In some embodiments, t is 2. In some embodiments, t is 3. In some embodiments, t is 4. In some embodiments, t is 5.

In some embodiments, ring A is a substituted or unsubstituted heterocycloalkylene. In some embodiments, ring A is a substituted or unsubstituted C₂-C₈heterocycloalkylene. In some embodiments, ring A is a substituted or unsubstituted monocyclic heterocycloalkylene, or substituted or unsubstituted bicyclic heterocycloalkylene. In some embodiments, ring A is a substituted or unsubstituted monocyclic heterocycloalkylene. In some embodiments, ring A is a substituted or unsubstituted monocyclic 5- or 6-membered heterocycloalkylene. In some embodiments, ring A is a substituted or unsubstituted monocyclic 5-membered heterocycloalkylene. In some embodiments, ring A is substituted or unsubstituted pyrrolidinyl, substituted or unsubstituted pyrrolidinonyl, substituted or unsubstituted tetrahydrofuranyl, substituted or unsubstituted tetrahydrofuranonyl, substituted or unsubstituted dihydrofuranonyl, substituted or unsubstituted dihydrofuranyl, substituted or unsubstituted tetrahydrothienyl, substituted or unsubstituted oxazolidinonyl, substituted or unsubstituted tetrahydropyranyl, substituted or unsubstituted dihydropyranyl, substituted or unsubstituted tetrahydrothiopyranyl, substituted or unsubstituted piperidinyl, substituted or unsubstituted morpholinyl, substituted or unsubstituted thiomorpholinyl, substituted or unsubstituted piperazinyl, substituted or unsubstituted aziridinyl, azetidinyl, substituted or unsubstituted oxetanyl, substituted or unsubstituted thietanyl, substituted or unsubstituted homopiperidinyl, substituted or unsubstituted oxepanyl, substituted or unsubstituted thiepanyl, substituted or unsubstituted oxazepinyl, substituted or unsubstituted diazepinyl, substituted or unsubstituted thiazepinyl, or substituted or unsubstituted 1,2,3,6-tetrahydropyridinyl. In some embodiments, ring A is a substituted or unsubstituted bicyclic heterocycloalkylene. In some embodiments, ring A is substituted or unsubstituted indolinyl, or substituted or unsubstituted indolinonyl.

In some embodiments, the compound of Formula (I) has the structure of Formula (III), or a pharmaceutically acceptable salt or solvate thereof:

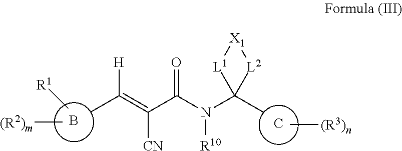

L¹ is —(C(R⁶)₂)ₚ—; p is 1, 2, 3, or 4;
L² is —(C(R⁶)₂)_{q}—; q is 0, 1, 2, 3, or 4;
X¹ is absent, —(C(R⁷)₂—, —O—, —S—, —S(O)—, —S(O)₂₋, —NR⁸—, —C(═O)—, —C(═O)NR⁴—, —C(═O)O—, —OC(═O)NR⁴—, or —NR⁴C(═O)NR⁴—;
   each R⁶ is independently selected from H, halogen, —OR⁴, substituted or unsubstituted alkyl, substituted or unsubstituted haloalkyl, substituted or unsubstituted phenyl, and substituted or unsubstituted heteroaryl;
   or two R⁶ on adjacent carbon atoms are taken together with the intervening atoms to form a monocyclic carbocycle or monocyclic heterocycle;
   each R⁷ is independently selected from H, halogen, —OR⁴, substituted or unsubstituted alkyl, substituted or unsubstituted haloalkyl, substituted or unsubstituted phenyl, and substituted or unsubstituted heteroaryl;
   or both R⁷ are taken together with the carbon atom to which they are attached to form a monocyclic carbocycle or monocyclic heterocycle;
   each R⁸ is independently selected from H, —S(═O)R⁵, —S(═O)₂R⁵, —S(═O)₂N(R⁴)₂, —C(═O)R⁵, —CO₂R⁵, or —C(═O)N(R⁴)₂.

In some embodiments, X¹ is absent, —(C(R⁷)₂—, —O—, —S—, —NR⁸—, or —C(═O)—.

In some embodiments, X¹ is absent.

In some embodiments, X¹ is —(C(R⁷)₂— or —C(═O)—.

In some embodiments, X¹ is —(C(R⁷)₂—; each R⁷ is independently selected from H, halogen, —O—C₁-C₆alkyl, —O—C₁-C₆haloalkyl, C₁-C₆alkyl, or C₁-C₆haloalkyl; or both R⁷ are taken together with the carbon atom to which they are attached to form a monocyclic carbocycle or monocyclic heterocycle.

In some embodiments, X¹ is —(C(R⁷)₂—; both R⁷ are taken together with the carbon atom to which they are attached to form a monocyclic carbocycle or monocyclic heterocycle.

In some embodiments, X¹ is —O— or —S.

In some embodiments, X¹ is —NR⁸—.

In some embodiments, each R⁸ is independently selected from H, —S(═O)₂R⁵, or —C(═O)R⁵.

In some embodiments, each R⁶ is independently selected from H, halogen, —O—C₁-C₆alkyl, —O—C₁-C₆haloalkyl, C₁-C₆alkyl, and C₁-C₆haloalkyl.

In some embodiments, each R⁶ is H.

In some embodiments, p is 1 or 2. In some embodiments, q is 1 or 2. In some embodiments, p is 1 or 2; and q is 1 or 2.

In some embodiments, ring B is phenyl.

In some embodiments, ring B is monocyclic heteroaryl.

In some embodiments, ring B is furanyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, or triazinyl.

In some embodiments, ring B is pyridinyl, pyrimidinyl, pyrazinyl, pyridazinyl, or triazinyl.

In some embodiments, ring is pyridinyl.

In some embodiments,

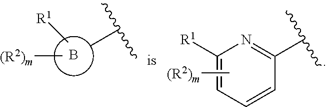

In some embodiments, ring B is furanyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, R¹ is selected from H, F, Cl, Br, —CN, substituted or unsubstituted alkyl, substituted or unsubstituted haloalkyl; each R² is independently selected from H, halogen, —OR⁴, —SR⁴, —N(R⁴)₂, —CN, —NO₂, substituted or unsubstituted alkyl, substituted or unsubstituted haloalkyl.

In some embodiments, R¹ is selected from H, F, Cl, or Br.

In some embodiments, ring C is phenyl, naphthyl, monocyclic heteroaryl or bicyclic heteroaryl.

In some embodiments, ring C is phenyl.

In some embodiments, ring C is monocyclic heteroaryl or bicyclic heteroaryl.

In some embodiments, ring C is monocyclic heteroaryl.

In some embodiments, ring C is furanyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, or triazinyl.

In some embodiments, ring C is furanyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, ring C is pyridinyl, pyrimidinyl, pyrazinyl, pyridazinyl, or triazinyl.

In some embodiments, ring C is bicyclic heteroaryl.

In some embodiments, ring C is quinolinyl, isoquinolinyl, quinazolinyl, quinoxalinyl, naphthyridinyl, indolyl, indazolyl, benzoxazolyl, benzisoxazolyl, benzofuranyl, benzothienyl, benzothiazolyl, benzimidazolyl, purinyl, cinnolinyl, phthalazinyl, pteridinyl, pyridopyrimidinyl, pyrazolopyrimidinyl, or azaindolyl.

In some embodiments, ring C is quinolinyl, isoquinolinyl, quinazolinyl, quinoxalinyl, indolyl, indazolyl, benzoxazolyl, benzisoxazolyl, benzofuranyl, benzothienyl, benzothiazolyl, or benzimidazolyl.

In some embodiments, each R³ is independently selected from H, halogen, —CN, —NO₂, —OR⁴, —SR⁴, —S(═O)R⁵, —S(═O)₂R⁵, —S(═O)₂N(R⁴)₂, —NR⁴S(═O)₂R⁵, —C(═O)R⁵, —OC(═O)R⁵, —CO₂R⁴, —OCO₂R⁵, —N(R⁴)₂, —C(═O)N(R⁴)₂, —OC(═O)N(R⁴)₂, —NHC(═O)R⁵, —NHC(═O)OR⁵, unsubstituted or substituted alkyl, unsubstituted or substituted fluroroalkyl, unsubstituted or substituted alkenyl, unsubstituted or substituted alkynyl, unsubstituted or substituted heteroalkyl, unsubstituted or substituted cycloalkyl, unsubstituted or substituted aryl, and unsubstituted or substituted heteroaryl.

In some embodiments, each R³ is independently selected from unsubstituted or substituted C₃-C₆cycloalkyl, unsubstituted or substituted phenyl, and unsubstituted or substituted monocyclic heteroaryl. In some embodiments, n is 1 and R³ is unsubstituted or substituted phenyl, unsubstituted or substituted monocyclic heteroaryl, or unsubstituted or substituted bicyclic heteroaryl. In some embodiments, n is 1 and R³ is unsubstituted or substituted phenyl. In some embodiments, n is 1 and R³ is unsubstituted or substituted monocyclic heteroaryl. In some embodiments, n is 1 and R³ is unsubstituted or substituted bicyclic heteroaryl.

In some embodiments, each R³ is independently selected from H, halogen, —CN, —NO₂, —OR⁴, —SR⁴, —S(═O)R⁵, —S(═O)₂R⁵, —S(═O)₂N(R⁴)₂, —NR⁴S(═O)₂R⁵, —C(═O)R⁵, —OC(═O)R⁵, —CO₂R⁴, —OCO₂R⁵, —N(R⁴)₂, —C(═O)N(R⁴)₂, —OC(═O)N(R⁴)₂, —NHC(═O)R⁵, —NHC(═O)OR⁵, C₁-C₆alkyl and C₁-C₆fluoroalkyl.

In some embodiments, each R³ is independently selected from H, halogen, —CN, —OR⁴, C₁-C₆alkyl and C₁-C₆fluroroalkyl.

In some embodiments, R³ is -L³-L⁴-L⁵-L⁶-R⁹; and n is 1.

In some embodiments, L³ is absent, —O—, —NR⁴—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —(CH₂)ᵣ—, or —(OCH₂CH₂)ᵣ—. In some embodiments, L³ is absent, —O—, —C(═O)NH—, —NHC(═O)—, —C(═O)O—, —OC(═O)—, —(CH₂)ᵣ—, or —(OCH₂CH₂)ᵣ—.

In some embodiments, L⁴ is absent, unsubstituted or substituted alkylene, unsubstituted or substituted heteroalkylene, unsubstituted or substituted alkenylene, unsubstituted or substituted alkynylene, or —(OCH₂CH₂)ₛ—, s is 1, 2, 3, or 4. In some embodiments, L⁴ is absent, or unsubstituted or substituted alkylene. In some embodiments, L⁴ is unsubstituted or substituted cycloalkylene, unsubstituted or substituted heterocycloalkylene, unsubstituted or substituted arylene, or unsubstituted or substituted heteroarylene.

In some embodiments, L⁵ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—. In some embodiments, L⁵ is absent, —O—, —NR⁴—, —C(═O)—, —C(═O)NH—, —NHC(═O)—, —C(═O)O—, or —OC(═O)—. In some embodiments, L⁵ is absent, —O—, —C(═O)—, —C(═O)NH—, —NHC(═O)—, —C(═O)O—, or —OC(═O)—.

In some embodiments, L⁶ is absent, unsubstituted or substituted alkylene, unsubstituted or substituted heteroalkylene. In some embodiments, L⁶ is absent, or unsubstituted or substituted alkylene.

In some embodiments, -L³-L⁴-L⁵-L⁶- is C₁-C₆alkylene, —O—C₁-C₆alkylene, —NH—C₁-C₆alkylene, —(OCH₂CH₂)ᵣ—, —C₁-C₆alkylene-OC(═O)—C₁-C₆alkylene-, —C₁-C₆alkylene-NHC(═O)—C₁-C₆alkylene-, —C(═O)—C₁-C₆alkylene-C(═O)—, —(CH₂)ᵣ—OC(═O)—C₁-C₆alkylene-, —(CH₂)ᵣ—NHC(═O)—C₁-C₆alkylene-, —CH═N—NH—C(═O)—C₁-C₆alkylene-, or —C(CH₃)═N—NH—C(═O)—C₁-C₆alkylene-.

In some embodiments, R⁹ is H, halogen, unsubstituted or substituted alkyl, unsubstituted or substituted alkenyl, unsubstituted or substituted alkynyl, or unsubstituted or substituted cycloalkyl. In some embodiments, R⁹ is unsubstituted or substituted aryl, or unsubstituted or substituted heteroaryl. In some embodiments, R⁹ is unsubstituted or substituted aryl. In some embodiments, R⁹ is unsubstituted or substituted heteroaryl.

In some embodiments, R⁹ is unsubstituted or substituted monocyclic heterocycloalkyl or unsubstituted or substituted bicyclic heterocycloalkyl.

In some embodiments, R⁹ is unsubstituted or substituted aziridinyl, unsubstituted or substituted azetidinyl, unsubstituted or substituted pyrrolidinyl, unsubstituted or substituted pyrrolidinonyl, unsubstituted or substituted oxazolidinonyl, unsubstituted or substituted piperidinyl, unsubstituted or substituted morpholinyl, unsubstituted or substituted thiomorpholinyl, unsubstituted or substituted piperazinyl, unsubstituted or substituted maleimidyl, or unsubstituted or substituted biotinyl. In some embodiments, R⁹ is unsubstituted or substituted maleimidyl.

In some embodiments,

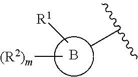

is B is as described in Table 1.

In some embodiments, ring L¹ is as described in Table 1.

In some embodiments, ring X¹ is as described in Table 1.

In some embodiments, ring L² is as described in Table 1.

In some embodiments, ring R³ is as described in Table 1 and/or Table 2.

In some embodiments, ring R¹⁰ is as described in Table 2.

In some embodiments, compounds described herein include those compounds described in the following tables:

TABLE 1

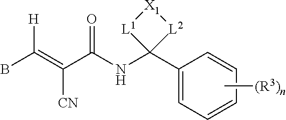

| Cmpd | B | —L¹— | X¹ | —L²— | n | R³ |
|---|---|---|---|---|---|---|
| 1 | 6-Bromopyridin-2-yl | —(CH₂)₂— | — | —(CH₂)₂— | 0 | H |
| 2 | 6-Bromopyridin-2-yl | —CH₂— | — | —CH₂— | 0 | H |
| 3 | 6-Bromopyridin-2-yl | —(CH₂)₂— | — | —CH₂— | 0 | H |
| 4 | 3,5-Dibromopyridin-2-yl | —(CH₂)₂— | — | —(CH₂)₂— | 0 | H |
| 5 | 3-Fluoro-5- | —(CH₂)₂— | — | —(CH₂)₂— | 0 | H |
| | chloropyridin-2-yl | | | | | |
| 6 | 3-Fluoro-6- | —(CH₂)₂— | — | —(CH₂)₂— | 0 | H |
| | bromopyridin-2-yl | | | | | |
| 7 | 3,6-Dichloropyridin-2-yl | —(CH₂)₂— | — | —(CH₂)₂— | 0 | H |
| 8 | 3-Fluoropyridin-2-yl | —(CH₂)₂— | — | —(CH₂)₂— | 0 | H |
| 9 | 5-Bromo-3- | —(CH₂)₂— | — | —(CH₂)₂— | 0 | H |
| | fluoropyridin-2-yl | | | | | |
| 10 | 3-Bromo-6- | —(CH₂)₂— | — | —(CH₂)₂— | 0 | H |
| | chloropyridin-2-yl | | | | | |
| 11 | 3,4-Dichlorophenyl | —(CH₂)₂— | — | —(CH₂)₂— | 0 | H |
| 12 | 4-Chloro-3-fluorophenyl | —(CH₂)₂— | — | —(CH₂)₂— | 0 | H |
| 13 | 4-Hydroxyphenyl | —(CH₂)₂— | — | —(CH₂)₂— | 0 | H |
| 14 | 3-Hydroxy-4- | —(CH₂)₂— | — | —(CH₂)₂— | 0 | H |
| | methoxyphenyl | | | | | |
| 15 | 6-Bromopyridin-2-yl | —(CH₂)₂— | — | —(CH₂)₂— | 1 | 4-Br |
| 16 | 6-Bromopyridin-2-yl | —(CH₂)₂— | — | —(CH₂)₂— | 1 | 4-OMe |
| 17 | 6-Bromopyridin-2-yl | —(CH₂)₃— | — | —(CH₂)₂— | 0 | H |
| 18 | 6-Bromopyridin-2-yl | —(CH₂)₂— | —O— | —(CH₂)₂— | 0 | H |
| 19 | 3,5-dibromopyridin-2-yl | —(CH₂)₂— | — | —(CH₂)₂— | 1 | 4-CF₃ |
| 20 | 3,5-dibromopyridin-2-yl | —(CH₂)₂— | — | —(CH₂)₂— | 1 | 3-Cl |
| 21 | 3,5-dibromopyridin-2-yl | —(CH₂)₂— | — | —(CH₂)₂— | 1 | 4-F |
| 22 | 3,5-dibromopyridin-2-yl | —(CH₂)₂— | — | —(CH₂)₂— | 1 | 4-Cl |
| 23 | 3,5-dibromopyridin-2-yl | —(CH₂)₃— | — | —(CH₂)₂— | 0 | H |
| 24 | 3,5-dibromopyridin-2-yl | —(CH₂)₂— | —O— | —(CH₂)₂— | 0 | H |
| 25 | 3,5-dibromopyridin-2-yl | —(CH₂)₃— | — | —(CH₂)₃— | 0 | H |
| 26 | 3,5-dibromopyridin-2-yl | —(CH₂)₂— | — | —(CH₂)₂— | 1 | 3-Me |
| 27 | 3,5-dibromopyridin-2-yl | —(CH₂)₂— | — | —(CH₂)₂— | 1 | 4-t-Bu |
| 28 | 4-Bromopyridin-2-yl | —(CH₂)₂— | — | —(CH₂)₂— | 0 | H |
| 29 | 5-Bromopyridin-2-yl | —(CH₂)₂— | — | —(CH₂)₂— | 0 | H |
| 30 | 2,3′-bipyridin-6-yl | —(CH₂)₂— | — | —(CH₂)₂— | 0 | H |
| 31 | 6-(4-(ethoxycarbonyl) | —(CH₂)₂— | — | —(CH₂)₂— | 0 | H |
| | phenyl)pyridin-2-yl | | | | | |
| 32 | 3,5-dibromopyridin-2-yl | —(CH₂)₂— | — | —(CH₂)₂— | 1 | 4-(ethoxycarbonyl)phenyl |
| 33 | 6-(5- | —(CH₂)₂— | — | —(CH₂)₂— | 0 | H |
| | methoxycarbonylpyridin- | | | | | |
| | 3-yl)pyridin-2-yl | | | | | |
| 34 | 6-(3-methoxycarbonyl- | —(CH₂)₂— | — | —(CH₂)₂— | 0 | H |
| | phenylyl)pyridin-2-yl | | | | | |
| 35 | [2,3′-bipyridin]-6-yl | —(CH₂)₂— | — | —(CH₂)₂— | 0 | H |
| 36 | 6-(4-(ethoxycarbonyl) | —(CH₂)₂— | — | —(CH₂)₂— | 0 | H |
| | phenyl)pyridin-2-yl | | | | | |
| 37 | 3,5-Dibromopyridin-2-yl | —(CH₂)₂— | — | —(CH₂)₂— | 0 | 4-[6-(methoxycarbonyl)pyridin-3- |
| | | | | | | yl] |
| 38 | 3,5-Dibromopyridin-2-yl | —(CH₂)₂— | — | —(CH₂)₂— | 0 | 4-[3-(methoxycarbonyl)phenyl] |
| 39 | 3,5-Dibromopyridin-2-yl | —(CH₂)₂— | — | —(CH₂)₂— | 0 | 4-(Pyridine-3-yl) |
| 40 | 6-(4-(carboxymethyl) | —(CH₂)₂— | — | —(CH₂)₂— | 0 | H |
| | phenyl)pyridin-2-yl | | | | | |
| 41 | 6-Bromopyridin-2-yl | —(CH₂)₂— | — | —(CH₂)₂— | 0 | 4-[4-(methoxycarbonyl)phenyl] |
| 42 | 6-((E)-3-methoxy-3- | —(CH₂)₂— | — | —(CH₂)₂— | 0 | H |
| | oxoprop-1-en-1- | | | | | |
| | yl)pyridin-2-yl | | | | | |
| 43 | 6-(3-(1H-pyrazol-1- | —(CH₂)₂— | — | —(CH₂)₂— | 0 | H |
| | yl)prop-1-yn-1- | | | | | |
| | yl)pyridin-2-yl | | | | | |
| 44 | 3,5-Dibromopyridin-2-yl | —(CH₂)₂— | — | —(CH₂)₂— | 0 | 4-(3-(1H-pyrazol-1-yl)prop-1-yn- |
| | | | | | | 1-yl |
| 45 | 6-((1- | —(CH₂)₂— | — | —(CH₂)₂— | 0 | H |
| | hydroxycyclopentyl) | | | | | |
| | ethynyl)pyridin-2-yl | | | | | |
| 46 | 3-Bromopyridin-2-yl | —(CH₂)₂— | — | —(CH₂)₂— | 0 | H |
| 47 | 2′-Ethoxy-5′- | —(CH₂)₂— | — | —(CH₂)₂— | 0 | H |
| | (trifluoromethyl)-[2,3′- | | | | | |
| | bipyridin]-6-yl | | | | | |
| 48 | 6-Bromopyridin-2-yl | —(CH₂)₂— | — | —(CH₂)₂— | 0 | 3-(methoxycarbonyl) |
| 49 | 5′-(tert-Butoxycarbonyl)- | —(CH₂)₂— | — | —(CH₂)₂— | 0 | H |
| | 6′-chloro-[2,3′- | | | | | |
| | bipyridin]-6-yl | | | | | |
| 50 | 3,5-Dibromopyridin-2-yl | —(CH₂)₂— | — | —(CH₂)₂— | 0 | 4-(3-morpholinoprop-1-yn-1-yl |
| 51 | 3,5-Dibromopyridin-2-yl | —(CH₂)₂— | — | —(CH₂)₂— | 0 | 4-(6-morpholinopyridin-3-yl |
| 52 | 3,5-Dibromopyridin-2-yl | —(CH₂)₂— | — | —(CH₂)₂— | 0 | 4-[3-(tert-butoxycarbonyl)phenyl] |
| 53 | 3,5-Dibromopyridin-2-yl | —(CH₂)₂— | — | —(CH₂)₂— | 0 | 4-(2-methoxypyridin-3-yl |
| 54 | 3,5-Dibromopyridin-2-yl | —(CH₂)₂— | — | —(CH₂)₂— | 0 | 4-(2-ethoxy-5- |
| | | | | | | (trifluoromethyl)pyridin-3-yl |
| 55 | 6-(3-Morpholinoprop-1- | —(CH₂)₂— | — | —(CH₂)₂— | 0 | H |
| | yn-1-yl)pyridin-2-yl | | | | | |
| 56 | 6-(3-(tert-Butoxy- | —(CH₂)₂— | — | —(CH₂)₂— | 0 | H |
| | carbonyl)phenyl)pyridin- | | | | | |
| | 2-yl | | | | | |
| 57 | 6′-Morpholino-[2,3′- | —(CH₂)₂— | — | —(CH₂)₂— | 0 | H |
| | bipyridin]-6-yl | | | | | |
| 58 | 6-Bromopyridin-2-yl | —(CH₂)₂— | — | —(CH₂)₂— | 0 | 3-Br |
| 59 | 3,5-Dibromopyridin-2-yl | —(CH₂)₂— | — | —(CH₂)₂— | 0 | 4-(1H-pyrrolo[2,3-b]pyridin-5-yl |
| 60 | 6-(3-(Ethoxycarbonyl)-2- | —(CH₂)₂— | — | —(CH₂)₂— | 0 | H |
| | fluorophenyl)pyridin-2- | | | | | |
| | yl | | | | | |
| 61 | 6-(1H-Pyrrolo[2,3- | —(CH₂)₂— | — | —(CH₂)₂— | 0 | H |
| | b]pyridin-5-yl)pyridin-2- | | | | | |
| | yl | | | | | |
| 62 | 6-(1-Methyl-1H-pyrazol- | —(CH₂)₂— | — | —(CH₂)₂— | 0 | H |
| | 4-yl)pyridin-2-yl | | | | | |
| 63 | 3,5-Dibromopyridin-2-yl | —(CH₂)₂— | — | —(CH₂)₂— | 0 | 4-(propen-2-yl) |
| 64 | 6′-(Trifluoromethyl)- | —(CH₂)₂— | — | —(CH₂)₂— | 0 | H |
| | [2,3′-bipyridin]-6-yl | | | | | |
| 65 | 6′-(Piperidin-1-yl)-[2,3′- | —(CH₂)₂— | — | —(CH₂)₂— | 0 | H |
| | bipyridin]-6-yl | | | | | |
| 66 | 3,5-Dibromopyridin-2-yl | —(CH₂)₂— | — | —(CH₂)₂— | 0 | 4-(dimethylcarbamoyl) |
| 67 | 6-Cyclopropylpyridin-2- | —(CH₂)₂— | — | —(CH₂)₂— | 0 | 4-(pyridine-3-yl) |
| | yl | | | | | |
| 68 | 3,5-Dibromopyridin-2-yl | —(CH₂)₂— | — | —(CH₂)₂— | 0 | 4-(6-(trifluoromethyl)pyridin-3-yl |
| 69 | 3,5-Dibromopyridin-2-yl | —(CH₂)₂— | — | —(CH₂)₂— | 0 | 4-(6-(piperidin-1-yl)pyridin-3-yl |
| 70 | 6-(1H-Indazol-5- | —(CH₂)₂— | — | —(CH₂)₂— | 0 | H |
| | yl)pyridin-2-yl | | | | | |
| 71 | 6-(2-carboxy-2- | —(CH₂)₂— | — | —(CH₂)₂— | 0 | H |
| | cyanovinyl)pyridin-2-yl | | | | | |
| 72 | 6-Bromopyridin-2-yl | —(CH₂)₂— | — | —(CH₂)₂— | 0 | 4-((4-methoxybenzyl)oxy) |
| 73 | 6-Bromopyridin-2-yl | —(CH₂)₂— | — | —(CH₂)₂— | 0 | 4-(pyridine-3-yl) |
| 74 | 6-Bromopyridin-2-yl | —(CH₂)₂— | — | —(CH₂)₂— | 0 | 4-(3-carboxyphenyl) |
| 75 | 6-Bromopyridin-2-yl | —(CH₂)₂— | — | —(CH₂)₂— | 0 | 4-((3-hydroxyoxetan-3-yl)ethynyl |
| 76 | 6-Bromopyridin-2-yl | —(CH₂)₂— | — | —(CH₂)₂— | 0 | 4-((1-hydroxycyclopentyl)ethynyl |
| 77 | 3,5-Dibromopyridin-2-yl | —(CH₂)₂— | — | —(CH₂)₂— | 0 | 4-(1-methyl-1H-pyrazol-4-yl |
| 78 | 6-Bromopyridin-2-yl | —(CH₂)₂— | — | —(CH₂)₂— | 0 | 4-(3-((2-hydroxyethyl)amino)prop- |
| | | | | | | 1-yn-1-yl |
| 79 | 6-Bromopyridin-2-yl | —(CH₂)₂— | — | —(CH₂)₂— | 0 | 4-OH |
| 80 | 6-Bromopyridin-2-yl | —(CH₂)₂— | O | —(CH₂)— | 0 | H |
| 81 | 6-Bromopyridin-2-yl | —(CH₂)₂— | — | —(CH₂)₂— | 0 | 3-hydroxymethyl |
| 82 | 6-Cyclopropylpyridin-2- | —(CH₂)₂— | — | —(CH₂)₂— | 0 | 4-(3-carboxyphenyl) |
| | yl | | | | | |
| 83 | 6-(pyrrolidin-1- | —(CH₂)₂— | — | —(CH₂)₂— | 0 | H |
| | yl)pyridin-2-yl | | | | | |
| 84 | 6-Bromopyridin-2-yl | —(CH₂)₂— | — | —(CH₂)₂— | 0 | 4-((tetradecanoyloxy)methyl |
| 85 | 6-Bromopyridin-2-yl | —(CH₂)₂— | — | —(CH₂)₂— | 0 | 4-(((6-(2,5-dioxo-2,5-dihydro-1H- |
| | | | | | | pyrrol-1-yl)hexanoyl)oxy)methyl |
| 86 | 6-Bromopyridin-2-yl | —(CH₂)₂— | — | —(CH₂)₂— | 0 | 4-hydroxymethyl |
| 87 | 6-Bromopyridin-2-yl | —(CH₂)₂— | — | —(CH₂)₂— | 0 | 4-(acetoxymethyl) |
| 88 | 6-Bromopyridin-2-yl | —(CH₂)₂— | — | —(CH₂)₂— | 0 | 4-(2-(tert-butoxy)-2-oxoethoxy) |
| 89 | 6-(2-fluoro-3- | —(CH₂)₂— | — | —(CH₂)₂— | 0 | H |
| | (carboxy)phenyl)pyridin- | | | | | |
| | 2-yl | | | | | |
| 90 | 6-Bromopyridin-2-yl | —(CH₂)₂— | — | —(CH₂)₂— | 0 | 4-(4-(carboxymethyl)phenyl) |
| 91 | 6-Bromopyridin-2-yl | —(CH₂)₂— | — | —(CH₂)₂— | 0 | 4-(4-(carboxy)phenyl) |
| 92 | 6-Cyclopropylpyridin-2- | —(CH₂)₂— | — | —(CH₂)₂— | 0 | 4-(4-(carboxy)phenyl) |
| | yl | | | | | |
| 93 | 6-Cyclopropylpyridin-2- | —(CH₂)₂— | — | —(CH₂)₂— | 0 | 4-(4-(carboxymethyl)phenyl) |
| | yl | | | | | |
| 94 | 6-Bromopyridin-2-yl | —(CH₂)₂— | — | —(CH₂)₂— | 0 | 4-(3- |
| | | | | | | ((phenylsulfonyl)carbamoyl)phenyl |
| 95 | 6-Bromopyridin-2-yl | —(CH₂)₂— | — | —(CH₂)₂— | 0 | 4-(1H-indazol-5-yl) |
| 96 | 6-Bromopyridin-2-yl | —(CH₂)₂— | — | —(CH₂)₂— | 0 | 4-(1H-indol-5-yl) |
| 97 | 6-(2-(2-ethoxy-2- | —(CH₂)₂— | — | —(CH₂)₂— | 0 | H |
| | oxoethyl)-1,2,3,4- | | | | | |
| | tetrahydroisoquinolin-7- | | | | | |
| | yl)pyridin-2-yl) | | | | | |
| 98 | 6-Bromopyridin-2-yl | —(CH₂)₂— | — | —(CH₂)₂— | 0 | 4-(3-(carboxymethyl)-4- |
| | | | | | | methylphenyl) |
| 99 | 6-Cyclopropylpyridin-2- | —(CH₂)₂— | — | —(CH₂)₂— | 0 | 4-(1H-indol-5-yl) |
| | yl | | | | | |

TABLE 2

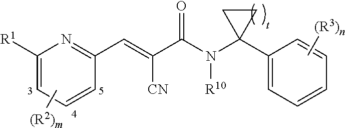

| Cmpd | R¹ | m | R² | t | n | R³ | R¹⁰ |
|---|---|---|---|---|---|---|---|
| 2-1 | Br | 0 | — | 1 | 0 | — | H |
| 2-2 | F | 0 | — | 1 | 0 | — | H |
| 2-3 | Cl | 0 | — | 1 | 0 | — | H |
| 2-4 | Br | 1 | 5-F | 1 | 0 | — | H |
| 2-5 | F | 1 | 5-F | 1 | 0 | — | H |
| 2-6 | Cl | 1 | 5-F | 1 | 0 | — | H |
| 2-7 | Br | 1 | 5-Cl | 1 | 0 | — | H |
| 2-8 | F | 1 | 5-Cl | 1 | 0 | — | H |
| 2-9 | Cl | 1 | 5-Cl | 1 | 0 | — | H |
| 2-10 | Br | 1 | 4-F | 1 | 0 | — | H |
| 2-11 | F | 1 | 4-F | 1 | 0 | — | H |
| 2-12 | Cl | 1 | 4-F | 1 | 0 | — | H |
| 2-13 | Br | 1 | 4-Cl | 1 | 0 | — | H |
| 2-14 | F | 1 | 4-Cl | 1 | 0 | — | H |
| 2-15 | Cl | 1 | 4-Cl | 1 | 0 | — | H |
| 2-16 | Br | 1 | 3-F | 1 | 0 | — | H |
| 2-17 | F | 1 | 3-F | 1 | 0 | — | H |
| 2-18 | Cl | 1 | 3-F | 1 | 0 | — | H |
| 2-19 | Br | 1 | 3-Cl | 1 | 0 | — | H |
| 2-20 | F | 1 | 3-Cl | 1 | 0 | — | H |
| 2-21 | Cl | 1 | 3-Cl | 1 | 0 | — | H |
| 2-22 | Br | 0 | — | 2 | 0 | — | H |
| 2-23 | F | 0 | — | 2 | 0 | — | H |
| 2-24 | Cl | 0 | — | 2 | 0 | — | H |
| 2-25 | Br | 1 | 5-F | 2 | 0 | — | H |
| 2-26 | F | 1 | 5-F | 2 | 0 | — | H |
| 2-27 | Cl | 1 | 5-F | 2 | 0 | — | H |
| 2-28 | Br | 1 | 5-Cl | 2 | 0 | — | H |
| 2-29 | F | 1 | 5-Cl | 2 | 0 | — | H |
| 2-30 | Cl | 1 | 5-Cl | 2 | 0 | — | H |
| 2-31 | Br | 1 | 4-F | 2 | 0 | — | H |
| 2-32 | F | 1 | 4-F | 2 | 0 | — | H |
| 2-33 | Cl | 1 | 4-F | 2 | 0 | — | H |
| 2-34 | Br | 1 | 4-Cl | 2 | 0 | — | H |
| 2-35 | F | 1 | 4-Cl | 2 | 0 | — | H |
| 2-36 | Cl | 1 | 4-Cl | 2 | 0 | — | H |
| 2-37 | Br | 1 | 3-F | 2 | 0 | — | H |
| 2-38 | F | 1 | 3-F | 2 | 0 | — | H |
| 2-39 | Cl | 1 | 3-F | 2 | 0 | — | H |
| 2-40 | Br | 1 | 3-Cl | 2 | 0 | — | H |
| 2-41 | F | 1 | 3-Cl | 2 | 0 | — | H |
| 2-42 | Cl | 1 | 3-Cl | 2 | 0 | — | H |
| 2-43 | Br | 0 | — | 3 | 0 | — | H |
| 2-44 | F | 0 | — | 3 | 0 | — | H |
| 2-45 | Cl | 0 | — | 3 | 0 | — | H |
| 2-46 | Br | 1 | 5-F | 3 | 0 | — | H |
| 2-47 | F | 1 | 5-F | 3 | 0 | — | H |
| 2-48 | Cl | 1 | 5-F | 3 | 0 | — | H |
| 2-49 | Br | 1 | 5-Cl | 3 | 0 | — | H |
| 2-50 | F | 1 | 5-Cl | 3 | 0 | — | H |
| 2-51 | Cl | 1 | 5-Cl | 3 | 0 | — | H |
| 2-52 | Br | 1 | 4-F | 3 | 0 | — | H |
| 2-53 | F | 1 | 4-F | 3 | 0 | — | H |
| 2-54 | Cl | 1 | 4-F | 3 | 0 | — | H |
| 2-55 | Br | 1 | 4-Cl | 3 | 0 | — | H |
| 2-56 | F | 1 | 4-Cl | 3 | 0 | — | H |
| 2-57 | Cl | 1 | 4-Cl | 3 | 0 | — | H |
| 2-58 | Br | 1 | 3-F | 3 | 0 | — | H |
| 2-59 | F | 1 | 3-F | 3 | 0 | — | H |
| 2-60 | Cl | 1 | 3-F | 3 | 0 | — | H |
| 2-61 | Br | 1 | 3-Cl | 3 | 0 | — | H |
| 2-62 | F | 1 | 3-Cl | 3 | 0 | — | H |
| 2-63 | Cl | 1 | 3-Cl | 3 | 0 | — | H |
| 2-64 | H | 2 | 3,5-diBr | 3 | 0 | — | H |
| 2-65 | H | 2 | 3,5-diBr | 3 | 0 | — | H |
| 2-66 | H | 2 | 3,5-diCl | 2 | 0 | — | H |
| 2-67 | H | 2 | 3,5-diCl | 3 | 0 | — | H |
| 2-68 | H | 2 | 3-Cl, 5-F | 2 | 0 | — | H |
| 2-69 | H | 2 | 3-Cl, 5-F | 3 | 0 | — | H |
| 2-70 | H | 2 | 3-Br, 5- | 2 | 0 | — | H |
| | | | F | | | | |
| 2-71 | H | 2 | 3-Br, 5- | 3 | 0 | — | H |
| | | | F | | | | |
| 2-72 | H | 2 | 3-F, 5-Cl | 2 | 0 | — | H |
| 2-73 | H | 2 | 3-F, 5-Cl | 3 | 0 | — | H |
| 2-74 | H | 2 | 3-F, 5- | 2 | 0 | — | H |
| | | | Br | | | | |
| 2-75 | H | 2 | 3-F, 5- | 3 | 0 | — | H |
| | | | Br | | | | |
| 2-76 | H | 2 | 3,5-DiF | 2 | 0 | — | H |
| 2-77 | H | 2 | 3,5-DiF | 3 | 0 | — | H |
| 2-78 | H | 2 | 3-Br, 5- | 2 | 0 | — | H |
| | | | Cl | | | | |
| 2-79 | H | 2 | 3-Br, 5- | 3 | 0 | — | H |
| | | | Cl | | | | |
| 2-80 | H | 2 | 3-Cl, 5- | 2 | 0 | — | H |
| | | | Br | | | | |
| 2-81 | H | 2 | 3-Cl, 5- | 3 | 0 | — | H |
| | | | Br | | | | |
| 2-82 | Br | 0 | — | 3 | 0 | — | CH₃ |
| 2-83 | H | 2 | 3,5-diBr | 3 | 0 | — | CH₃ |
| 2-84 | H | 2 | 3,5-diCl | 3 | 0 | — | CH₃ |
| 2-85 | H | 2 | 3-Br, 5- | 3 | 0 | — | CH₃ |
| | | | F | | | | |
| 2-86 | Br | 0 | — | 4 | 0 | — | CH₃ |
| 2-87 | H | 2 | 3,5-diBr | 4 | 0 | — | CH₃ |
| 2-88 | H | 2 | 3,5-diCl | 4 | 0 | — | CH₃ |
| 2-89 | H | 2 | 3-Br, 5- | 4 | 0 | — | CH₃ |
| | | | F | | | | |
| 2-90 | Br | 0 | — | 3 | 0 | — | CH₂CH₃ |
| 2-91 | H | 2 | 3,5-diBr | 3 | 0 | — | CH₂CH₃ |
| 2-92 | H | 2 | 3,5-diCl | 3 | 0 | — | CH₂CH₃ |
| 2-93 | H | 2 | 3-Br, 5- | 3 | 0 | — | CH₂CH₃ |
| | | | F | | | | |
| 2-94 | Br | 0 | — | 4 | 0 | — | CH₂CH₃ |
| 2-95 | H | 2 | 3,5-diBr | 4 | 0 | — | CH₂CH₃ |
| 2-96 | H | 2 | 3,5-diCl | 4 | 0 | — | CH₂CH₃ |
| 2-97 | H | 2 | 3-Br, 5- | 4 | 0 | — | CH₂CH₃ |
| | | | F | | | | |
| 2-98 | H | 2 | 3,5-diBr | 3 | 0 | — | Pyridine-3-ylmethyl |

TABLE 3

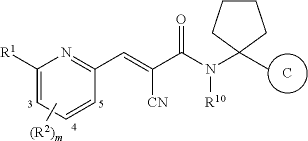

| Cmpd | R¹ | m | R² | C |
|---|---|---|---|---|
| 3-1 | Br | 0 | — | Pyridin-3-yl |
| 3-2 | Br | 0 | — | Pyridin-4-yl |
| 3-3 | H | 2 | 3,5-DiBr | Pyridin-4-yl |
| 3-4 | 3-(1H-pyrazol-1- | 0 | — | Pyridin-4-yl |
| | yl)prop-1-yn-1-yl | | | |
| 3-5 | (1-hydroxy- | 0 | — | Pyridin-4-yl |
| | cyclopentyl) | | | |
| | ethynyl | | | |
| 3-6 | Br | 0 | — | 2-Chloropyridin-4-yl |
| 3-7 | Br | 0 | — | 2-Fluoropyridin-4-yl |

Any combination of the groups described above for the various variables is contemplated herein. Throughout the specification, groups and substituents thereof are chosen by one skilled in the field to provide stable moieties and compounds.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviours. Also, because the salt-forming molecule can be in equilibrium with a neutral form, passage through biological membranes can be adjusted.

In some embodiments, pharmaceutically acceptable salts are obtained by reacting a compound described herein with an acid. In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt. In some embodiments, a compound described herein is prepared as a hydrochloride salt.

In some embodiments, pharmaceutically acceptable salts are obtained by reacting a compound described herein with a base. In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt. In some embodiments, the compounds provided herein are prepared as a sodium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

### Synthesis of Compounds

Compounds described herein are synthesized using standard synthetic techniques or using methods known in the art in combination with methods described herein.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Compounds described herein may be prepared in a variety of ways. In some embodiments, as shown in Scheme I, the amine of general structure I-1 is coupled with acid I-2 using standard amide bond forming reactions (for example, using HATU with an organic base, such as DIEA, in a solvent, such as DMF) to yield the α-cyanoamide I-4. In other embodiments, acid chloride I-3 is reacted with I-1 in the presence of an organic base, such as triethylamine, to provide I-4. In some embodiments, condensation of I-4 with an appropriate aldehyde I-5 in the presence of a catalyst, for example, β-alanine or piperidine in aqueous alcohol then provides the target cyano acrylamide I.

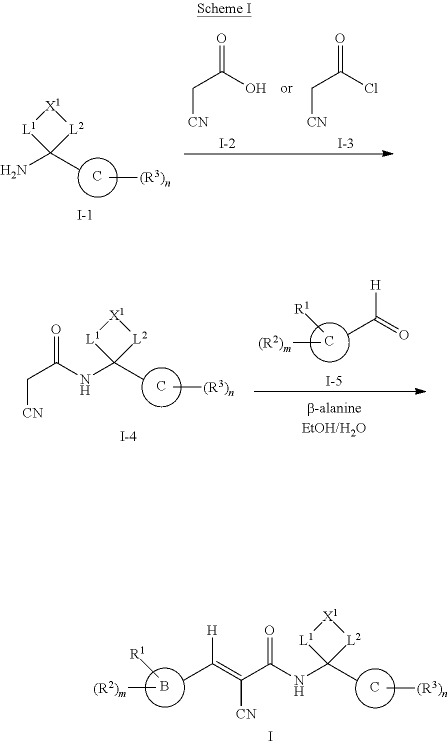

The amines of general structure I-1 may be prepared in a variety of ways. In some embodiments, the amines of general structure I-1 are cyclo(hetero)alkylbenzylamines or cyclo(hetero)alkylheteroarylmethylamines. In other embodiments, the amines of general structure I-1 are commercially available. In other embodiments, the amines of general structure I-1 are readily prepared by chemical synthesis using standard methodologies. Examples of such methodologies are described Schemes II, III and IV and are not meant to be limiting.

In some embodiments, amines of general structure I-1 are prepared as outlined in Scheme II.

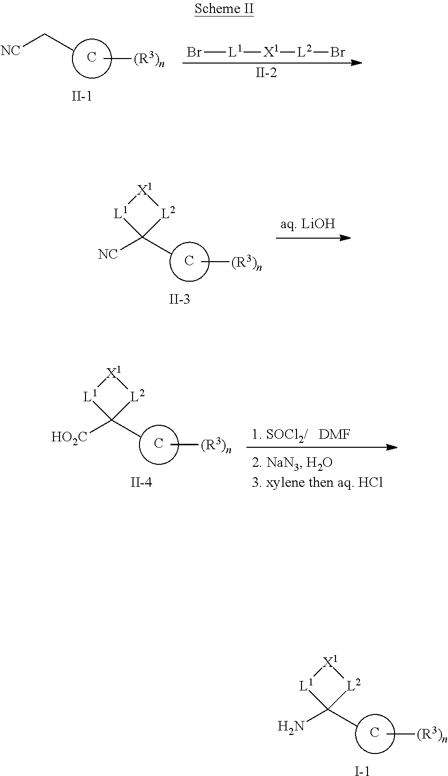

Benzylnitriles and heteroarylmethylnitriles of general structure II-1 are commercially available or can be synthesized. In other embodiments, the compounds of general structure II-1 are obtained by displacement of the corresponding benylhalide with NaCN. In some embodiments, the compound of general structure II-1 is alkylated with a bis-functionalized electrophile II-2) to yield the cyclo(hetero)alkyl derivative II-3. In other embodiments, the hydrolysis of the nitrile of II-3 using, for example aqueous LiOH in a polar organic solvent such as THF and EtOH, affords the acid II-4. In some embodiments, the acid II-4 is converted to the amine I-1 by the following sequence involving (a) conversion of the acid to an acid chloride (using e.g. SOCl₂ in DMF) then (b) treatment with NaN3 in water to generate the acyl azide followed by (c) thermal rearrangement in a high boiling solvent such as xylene or t-butanol/toluene and acid work-up (e.g. with 3N HCl) to afford the amine I-1 (see Taveras et al, patent application WO2005/68460 p 243).

In some embodiments, the compounds of general structure I-1 are prepared via the synthetic route shown in Scheme III (see for example Thurkauf et al, J. Med. Chem., 1990, 33, p 1452-1458).

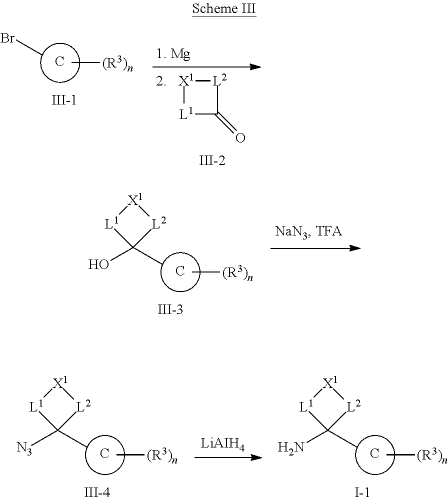

In other embodiments, a Grignard reagent prepared from bromo (or iodo) compound III-1 is condensed with a cyclic ketone III-2 to afford the tertiary alcohol III-3. In some embodiments, the treatment of the alcohol III-3 with NaN₃ and TFA in an organic solvent, such as CHCl₃ then yields the tertiary azide III-4. In other embodiments, the reduction of the azide III-4 using, for example LiAlH₄ in a solvent such as THF then yields the amine I-1.

In other embodiments, amines of general structure I-1 are prepared from the synthetic route shown in Scheme IV.

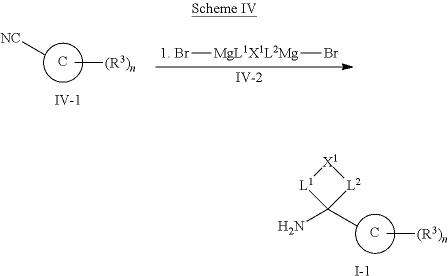

In some embodiments, the treatment of aryl nitrile IV-I with bis-Grignard reagent IV-2 in a solvent such as diethyl ether or THF affords the amine of general structure I-I.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkelene is a C₁-C₆alkylene. In other embodiments, an alkylene is a C₁-C₄alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, —CH₂CH₂CH₂CH₂CH₂—, —CH₂CH₂CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic.

“Bicyclic” refers to two fused rings. Fusion of the rings can occur in three ways: across a bond between two atoms, across a sequence of atoms (bridgehead), or at a single atom (spirocyclic, forming a spiro compound).

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoralkyl is a C₁-C₆fluoroalkyl.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic. Bicyclic heterocycles include two fused rings. Fusion of the rings can occur in three ways: across a bond between two atoms, across a sequence of atoms (bridgehead), or at a single atom (spirocyclic, forming a spiro compound).

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicycicic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s). In some embodiments, optional substituents are individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CF₃, —CH₂CF₃, —OCH₃, —OCH₂CH₃, —OCF₃ and —OCH₂CF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist. In some embodiments, a modulator is a degrader.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from inhibition or reduction of the activity of one or more deubiquitinase enzymes. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents. In certain embodiments, the pharmaceutical composition further comprises one or more anti-cancer agents. In further or alternative embodiments, the pharmaceutical composition further comprises one or more anti-cancer agents bound to human serum albumin e.g. Abraxane.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, a compound described herein, or a pharmaceutically acceptable salt thereof, is administered in combination with chemotherapy, hormone blocking therapy, radiation therapy, monoclonal antibodies, or combinations thereof.

Chemotherapy includes the use of anti-cancer agents.

### EXAMPLES

The following examples are provided for illustrative purposes only and not to limit the scope of the claims provided herein.

### Example 1: Preparation of (E)-3-(6-Bromopyridin-2-yl)-2-cyano-N-(1-phenylcyclopentyl)acrylamide (Compound 1)

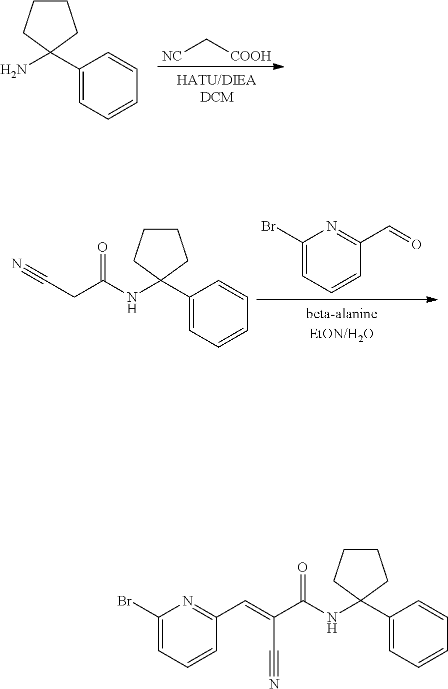

To a mixture of cyanoacetic acid (43 mg, 0.5 mmol) and 1-[bis(dimethylamino)methylene]-1H-1,2,3-triazolo[4,5-b]pyridinium 3-oxid hexafluorophosphate (HATU) (190 mg, 0.5 mmol) in dichloromethane (5 mL) was added 1-phenylcyclopentan-1-amine (81 mg, 0.5 mmol), followed by N,N-diisopropylethylamine (174 uL, 1.0 mmol). The mixture was stirred at room temperature for overnight. The mixture was then evaporated to dryness. The residue was extracted with ethyl acetate and distilled water. The organic layer was separated, washed with brine, dried over Na₂SO₄ and concentrated to dryness. The residue was re-dissolved in a mixture of ethanol (2 mL) and water (2 mL). To the mixture was then added β-alanine (356 mg, 4.0 mmol), followed by 6-bromo-2-pyridinecarboxaldehyde (186 mg, 1.0 mmol). The resulting mixture was then stirred at room temperature overnight and a white precipitate formed. The product was then collected via filtration, and rinsed with a solution of 1:1 ethanol:water 3× to afford (E)-3-(6-bromopyridin-2-yl)-2-cyano-N-(1-phenylcyclopentyl)acrylamide (Compound 1) (94 mg, 47%) as a white solid. ¹H NMR (DMSO-d6): δ 1.77 (m, 4H), 1.98 (m, 2H), 2.41 (m, 2H), 7.20 (t, 1H), 7.29 (t, 2H), 7.39 (d, 2H), 7.79 (d, 1H), 7.83-7.98 (m, 3H), 8.67 (s, 1H).

### Example 2: Preparation of (E)-3-(6-Bromopyridin-2-yl)-2-cyano-N-(1-phenylcyclopropyl)acrylamide (Compound 2)

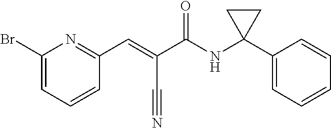 [SMILES: N#C/C(=C\c1cccc(Br)n1)C(=O)NC1(c2ccccc2)CC1]

Following the procedure for Example 1, but using 1-phenylcyclopropanamine hydrochloride in place of 1-phenylcyclopentan-1-amine, the title compound was prepared. MS (ES+) m/z 368 (M+H)⁺.

### Example 3: Preparation of (E)-3-(6-Bromopyridin-2-yl)-2-cyano-N-(1-phenylcyclobutyl)acrylamide (Compound 3)

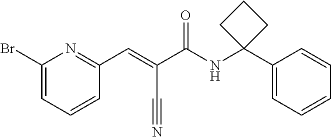 [SMILES: N#C/C(=C\c1cccc(Br)n1)C(=O)NC1(c2ccccc2)CCC1]

Following the procedure for Example 1, but using 1-phenylcyclobutanamine hydrochloride in place of 1-phenylcyclopentan-1-amine, the title compound was prepared. MS (ES+) m/z 382 (M+H)⁺.

### Example 4: Preparation of (E)-3-(3,5-Dibromopyridin-2-yl)-2-cyano-N-(1-phenylcyclopentyl)acrylamide (Compound 4)

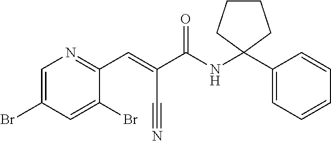 [SMILES: N#C/C(=C\c1ncc(Br)cc1Br)C(=O)NC1(c2ccccc2)CCCC1]

Following the procedure for Example 1, but using 3,5-dibromopicolinaldehyde in place of 6-bromo-2-pyridinecarboxaldehyde, the title compound was prepared. 1H NMR (DMSO-d6): δ 1.75 (m, 4H), 1.96 (m, 2H), 2.41 (m, 2H), 7.20 (t, 1H), 7.30 (t, 2H), 7.39 (d, 2H), 8.10 (s, 1H), 8.67 (s, 1H), 8.72 (s, 1H), 8.88 (s, 1H).

### Example 5: Preparation of (E)-3-(3-Fluoro-5-chloropyridin-2-yl)-2-cyano-N-(1-phenylcyclopentyl)acrylamide (Compound 5)

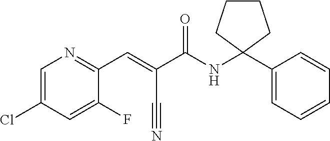 [SMILES: N#C/C(=C\c1ncc(Cl)cc1F)C(=O)NC1(c2ccccc2)CCCC1]

Following the procedure for Example 1, but using 5-chloro-3-fluoropicolinaldehyde in place of 6-bromo-2-pyridinecarboxaldehyde, the title compound was prepared. ¹H NMR (DMSO-d6): δ 1.75 (m, 4H), 1.96 (m, 2H), 2.41 (m, 2H), 7.20 (t, 1H), 7.30 (t, 2H), 7.39 (d, 2H), 8.00 (s, 1H), 8.30 (d, 1H), 8.72 (d, 2H).

### Example 6: Preparation of (E)-3-(3-Fluoro-6-bromopyridin-2-yl)-2-cyano-N-(1-phenylcyclopentyl)acrylamide (Compound 6)

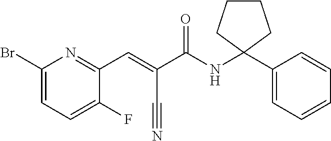 [SMILES: N#C/C(=C\c1nc(Br)ccc1F)C(=O)NC1(c2ccccc2)CCCC1]

Following the procedure for Example 1, but using 6-bromo-3-fluoropicolinaldehyde in place of 6-bromo-2-pyridinecarboxaldehyde, the title compound was prepared. ¹H NMR (DMSO-d6): δ 1.75 (m, 4H), 1.96 (m, 2H), 2.41 (m, 2H), 7.18 (t, 1H), 7.29 (t, 2H), 7.86-8.01 (m, 3H), 8.75 (s, 1H).

### Example 7: Preparation of (E)-3-(3,6-Dichloropyridin-2-yl)-2-cyano-N-(1-phenylcyclopentyl)acrylamide (Compound 7)

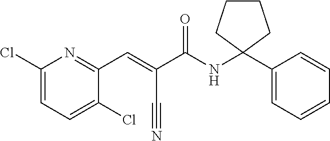 [SMILES: N#C/C(=C\c1nc(Cl)ccc1Cl)C(=O)NC1(c2ccccc2)CCCC1]

Following the procedure for Example 1, but using 3,6-dichloropicolinaldehyde in place of 6-bromo-2-pyridinecarboxaldehyde, the title compound was prepared. ¹H NMR (DMSO-d6): δ 1.75 (m, 4H), 1.96 (m, 2H), 2.41 (m, 2H), 7.18 (t, 1H), 7.29 (t, 2H), 7.39 (d, 2H), 7.73 (d, 1H), 8.06 (s, 1H), 8.18 (d, 1H), 8.75 (s, 1H).

### Example 8: Preparation of (E)-3-(3-Fluoropyridin-2-yl)-2-cyano-N-(1-phenylcyclopentyl)acrylamide (Compound 8)

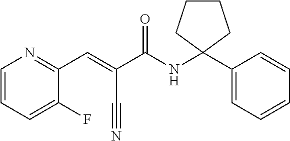 [SMILES: N#C/C(=C\c1ncccc1F)C(=O)NC1(c2ccccc2)CCCC1]

Following the procedure for Example 1, but using 3-fluoro-2-pyridinecarboxaldehyde in place of 6-bromo-2-pyridinecarboxaldehyde, the title compound was prepared. ¹H NMR (DMSO-d6): δ 1.75 (m, 4H), 1.96 (m, 2H), 2.41 (m, 2H), 7.18 (t, 1H), 7.29 (t, 2H), 7.39 (d, 2H), 7.67 (m, 1H), 7.92 (t, 1H), 8.04 (s, 1H), 8.61 (d, 1H), 8.71 (s, 1H).

### Example 9: Preparation of (E)-3-(5-Bromo-3-fluoropyridin-2-yl)-2-cyano-N-(1-phenylcyclopentyl)acrylamide (Compound 9)

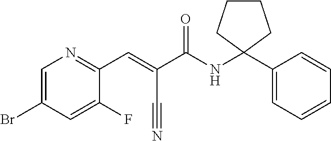 [SMILES: N#C/C(=C\c1ncc(Br)cc1F)C(=O)NC1(c2ccccc2)CCCC1]

Following the procedure for Example 1, but using 5-bromo-3-fluoropicolinaldehyde in place of 6-bromo-2-pyridinecarboxaldehyde, the title compound was prepared. ¹H NMR (DMSO-d6): δ 1.75 (m, 4H), 1.96 (m, 2H), 2.41 (m, 2H), 7.18 (t, 1H), 7.29 (t, 2H), 7.39 (d, 2H), 7.99 (s, 1H), 8.41 (d, 1H), 8.66-8.84 (m, 2H).

### Example 10: Preparation of (E)-3-(3-Bromo-6-chloropyridin-2-yl)-2-cyano-N-(1-phenylcyclopentyl)acrylamide (Compound 10)

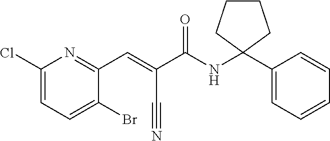 [SMILES: N#C/C(=C\c1nc(Cl)ccc1Br)C(=O)NC1(c2ccccc2)CCCC1]

Following the procedure for Example 1, but using 3-bromo-6-chloropyridine-2-carboxaldehyde in place of 6-bromo-2-pyridinecarboxaldehyde, the title compound was prepared. ¹H NMR (DMSO-d6): δ 1.75 (m, 4H), 1.96 (m, 2H), 2.33 (m, 2H), 7.19 (t, 1H), 7.29 (t, 2H), 7.37 (d, 2H), 7.51 (d, 1H), 8.02 (s, 1H), 8.20 (d, 1H), 8.73 (s, 1H).

### Example 11: Preparation of (E)-3-(3,4-Dichlorophenyl)-2-cyano-N-(1-phenylcyclopentyl)acrylamide (Compound 11)

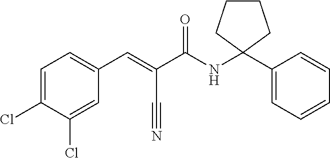 [SMILES: N#C/C(=C\c1ccc(Cl)c(Cl)c1)C(=O)NC1(c2ccccc2)CCCC1]

Following the procedure for Example 1, but using 3,4-dichlorobenzaldehyde in place of 6-bromo-2-pyridinecarboxaldehyde, the title compound was prepared. ¹H NMR (DMSO-d6): δ 1.75 (m, 4H), 1.96 (m, 2H), 2.33 (m, 2H), 7.19 (t, 1H), 7.29 (t, 2H), 7.37 (d, 2H), 7.85 (d, 1H), 7.92 (d, 1H), 8.04 (s, 1H), 8.15 (s, 1H), 8.61 (s, 1H).

### Example 12: Preparation of (E)-3-(4-Chloro-3-fluorophenyl)-2-cyano-N-(1-phenylcyclopentyl)acrylamide (Compound 12)

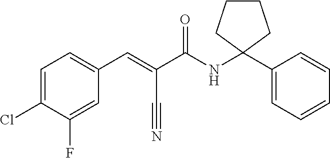 [SMILES: N#C/C(=C\c1ccc(Cl)c(F)c1)C(=O)NC1(c2ccccc2)CCCC1]

Following the procedure for Example 1, but using 4-chloro-3-fluorobenzaldehyde in place of 6-bromo-2-pyridinecarboxaldehyde, the title compound was prepared. ¹H NMR (DMSO-d6): δ 1.75 (m, 4H), 1.96 (m, 2H), 2.38 (m, 2H), 7.10-7.50 (m, 5H), 7.75-7.98 (m, 3H), 8.04 (s, 1H), 8.61 (s, 1H).

### Example 13: Preparation of (E)-3-(4-Hydroxyphenyl)-2-cyano-N-(1-phenylcyclopentyl)acrylamide (Compound 13)

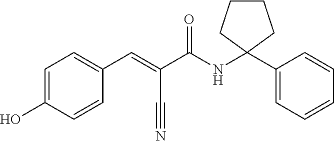 [SMILES: N#C/C(=C\c1ccc(O)cc1)C(=O)NC1(c2ccccc2)CCCC1]

Following the procedure for Example 1, but using p-hydroxybenzaldehyde in place of 6-bromo-2-pyridinecarboxaldehyde, the title compound was prepared. MS (ES+) m/z 355 (M+Na)⁺.

### Example 14: Preparation of (E)-3-(3-Hydroxy-4-methoxyphenyl)-2-cyano-N-(1-phenylcyclopentyl)acrylamide (Compound 14)

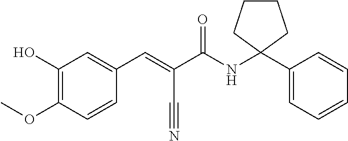 [SMILES: COc1ccc(/C=C(\C#N)C(=O)NC2(c3ccccc3)CCCC2)cc1O]

Following the procedure for Example 1, but using 3-hydroxy-4-methoxybenzaldehyde in place of 6-bromo-2-pyridinecarboxaldehyde, the title compound was prepared. MS (ES+) m/z 385 (M+Na)⁺.

### Example 15: Preparation of (E)-N-(1-(4-bromophenyl)cyclopentyl)-3-(6-bromopyridin-2-yl)-2-cyanoacrylamide (Compound 15)

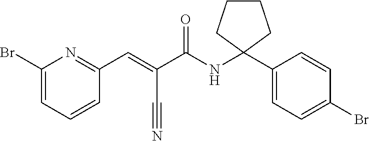 [SMILES: N#C/C(=C\c1cccc(Br)n1)C(=O)NC1(c2ccc(Br)cc2)CCCC1]

Following the procedure for Example 1, but using 1-(4-bromophenyl)cyclopentan-1-amine hydrochloride in place of 1-phenylcyclopentan-1-amine, the title compound was prepared. ¹H NMR (DMSO-d6): δ 1.75 (m, 4H), 1.96 (m, 2H), 2.38 (m, 2H), 7.34 (d, 2H), 7.48 (d, 2H), 7.78 (d, 1H), 7.86 (d, 1H), 7.90-7.96 (m, 2H).

### Example 16: Preparation of (E)-3-(6-Bromopyridin-2-yl)-2-cyano-N-(1-(4-methoxyphenyl)cyclopentyl)acrylamide (Compound 16)

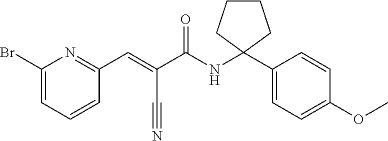 [SMILES: COc1ccc(C2(NC(=O)/C(C#N)=C/c3cccc(Br)n3)CCCC2)cc1]

Following the procedure for Example 1, but using 1-(4-methoxyphenyl)cyclopentan-1-amine hydrochloride in place of 1-phenylcyclopentan-1-amine, the title compound was prepared. MS (ES+) m/z 448 (M+Na)⁺.

### Example 17: Preparation of (E)-3-(6-Bromopyridin-2-yl)-2-cyano-N-(1-phenylcyclohexyl)acrylamide (Compound 17)

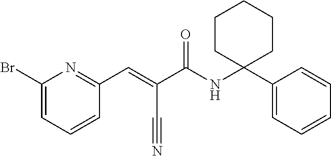 [SMILES: N#C/C(=C\c1cccc(Br)n1)C(=O)NC1(c2ccccc2)CCCCC1]

Following the procedure for Example 1, but using 1-phenylcyclohexane-1-amine in place of 1-phenylcyclopentan-1-amine, the title compound was prepared. ¹H NMR (DMSO-d6): δ 1.15-1.85 (m, 8H), 2.40 (m, 2H), 7.19 (t, 1H), 7.30 (t, 2H), 7.40 (d, 2H), 7.79 (d, 1H), 7.85-8.02 (m, 3H), 8.37 (s, 1H).

### Example 18: Preparation of (E)-3-(6-Bromopyridin-2-yl)-2-cyano-N-(4-phenyltetrahydro-2H-pyran-4-yl)acrylamide (Compound 18)

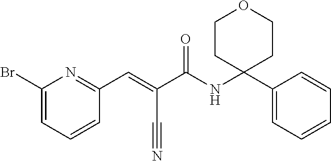 [SMILES: N#C/C(=C\c1cccc(Br)n1)C(=O)NC1(c2ccccc2)CCOCC1]

Following the procedure for Example 1, but using 4-phenyloxan-4-amine in place of 1-phenylcyclopentan-1-amine, the title compound was prepared. ¹H NMR (DMSO-d6): δ 1.96 (m, 2H), 2.40 (m, 2H), 3.63-3.86 (m, 4H), 7.23 (t, 1H), 7.33 (t, 2H), 7.41 (d, 2H), 7.79 (d, 1H), 7.86-7.97 (m, 2H), 8.01 (s, 1H), 8.71 (s, 1H).

### Example 19: Preparation of (E)-2-Cyano-3-(3,5-dibromopyridin-2-yl)-N-(1-(4-(trifluoromethyl)phenyl) cyclopentyl) acrylamide (Compound 19)

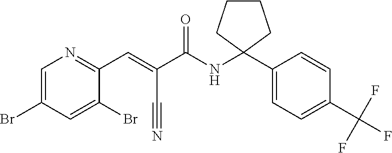 [SMILES: N#C/C(=C\c1ncc(Br)cc1Br)C(=O)NC1(c2ccc(C(F)(F)F)cc2)CCCC1]

Following the procedure for Example 1, but using 3,5-dibromopicolinaldehyde in place of 6-bromo-2-pyridinecarboxaldehyde and using 1-[4-(trifluoromethyl)phenyl]cyclopentan-1-amine in place of 1-phenylcyclopentan-1-amine, the title compound was prepared. ¹H NMR (DMSO-d6): δ 1.78 (m, 4H), 2.01 (m, 2H), 2.42 (m, 2H), 7.60 (d, 2H), 7.68 (d, 2H), 8.13 (s, 1H), 8.67 (d, 1H), 8.87-8.90 (m, 2H).

### Example 20: Preparation of (E)-N-(1-(3-Chlorophenyl)cyclopentyl)-2-cyano-3-(3,5-dibromopyridin-2-yl)acrylamide (Compound 20)

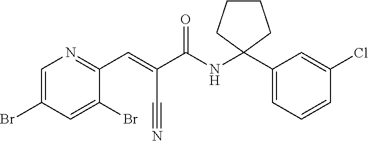 [SMILES: N#C/C(=C\c1ncc(Br)cc1Br)C(=O)NC1(c2cccc(Cl)c2)CCCC1]

Following the procedure for Example 1, but using 3,5-dibromopicolinaldehyde in place of 6-bromo-2-pyridinecarboxaldehyde and using 1-(3-chlorophenyl)cyclopentan-1-amine in place of 1-phenylcyclopentan-1-amine, the title compound was prepared. ¹H NMR (DMSO-d6): δ 1.78 (m, 4H), 1.98 (m, 2H), 2.42 (m, 2H), 7.22-7.39 (m, 3H), 7.42 (s, 1H), 8.12 (s, 1H), 8.67 (s, 1H), 8.77 (s, 1H), 8.88 (s, 1H).

### Example 21: Preparation of (E)-2-Cyano-3-(3,5-dibromopyridin-2-yl)-N-(1-(4-fluorophenyl)cyclopentyl)acrylamide (Compound 21)

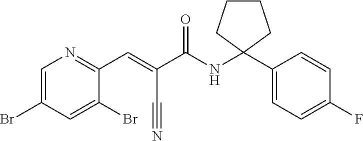 [SMILES: N#C/C(=C\c1ncc(Br)cc1Br)C(=O)NC1(c2ccc(F)cc2)CCCC1]

Following the procedure for Example 1, but using 3,5-dibromopicolinaldehyde in place of 6-bromo-2-pyridinecarboxaldehyde and using 1-(4-fluorophenyl)cyclopentan-1-amine hydrochloride in place of 1-phenylcyclopentan-1-amine, the title compound was prepared. ¹H NMR (DMSO-d6): δ 1.78 (m, 4H), 1.98 (m, 2H), 2.42 (m, 2H), 7.12 (t, 2H), 7.43 (t, 2H), 8.10 (s, 1H), 8.67 (s, 1H), 8.72 (s, 1H), 8.87 (s, 1H).

### Example 22: Preparation of (E)-N-(1-(4-Chlorophenyl)cyclopentyl)-2-cyano-3-(3,5-dibromopyridin-2-yl)acrylamide (Compound 22)

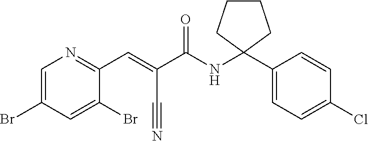 [SMILES: N#C/C(=C\c1ncc(Br)cc1Br)C(=O)NC1(c2ccc(Cl)cc2)CCCC1]

Following the procedure for Example 1, but using 3,5-dibromopicolinaldehyde in place of 6-bromo-2-pyridinecarboxaldehyde and using 1-(4-chlorophenyl)cyclopentan-1-amine hydrochloride in place of 1-phenylcyclopentan-1-amine, the title compound was prepared. ¹H NMR (DMSO-d6): δ 1.76 (m, 4H), 1.98 (m, 2H), 2.42 (m, 2H), 7.31-7.44 (m, 4H), 8.10 (s, 1H), 8.67 (s, 1H), 8.77 (s, 1H), 8.87 (s, 1H).

### Example 23: Preparation of (E)-2-Cyano-3-(3,5-dibromopyridin-2-yl)-N-(1-phenylcyclohexyl)acrylamide (Compound 23)

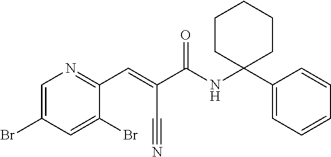 [SMILES: N#C/C(=C\c1ncc(Br)cc1Br)C(=O)NC1(c2ccccc2)CCCCC1]

Following the procedure for Example 1, but using 3,5-dibromopicolinaldehyde in place of 6-bromo-2-pyridinecarboxaldehyde and using 1-phenylcyclohexane-1-amine in place of 1-phenylcyclopentan-1-amine, the title compound was prepared. ¹H NMR (DMSO-d6): δ 1.15-1.85 (m, 8H), 2.43 (m, 2H), 7.19 (t, 1H), 7.30 (t, 2H), 7.40 (d, 2H), 8.13 (s, 1H), 8.42 (s, 1H), 8.67 (s, 1H), 8.89 (s, 1H).

### Example 24: Preparation of (E)-2-Cyano-3-(3,5-dibromopyridin-2-yl)-N-(4-phenyltetrahydro-2H-pyran-4-yl)acrylamide (Compound 24)

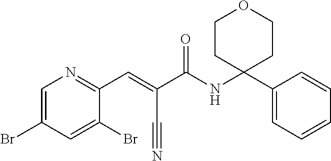 [SMILES: N#C/C(=C\c1ncc(Br)cc1Br)C(=O)NC1(c2ccccc2)CCOCC1]

Following the procedure for Example 1, but using 3,5-dibromopicolinaldehyde in place of 6-bromo-2-pyridinecarboxaldehyde and using 4-phenyloxan-4-amine in place of 1-phenylcyclopentan-1-amine, the title compound was prepared. ¹H NMR (DMSO-d6): δ 1.97 (t, 2H), 2.40 (m, 2H), 3.66 (t, 2H), 3.80 (m, 2H), 7.23 (t, 1H), 7.33 (t, 2H), 7.41 (d, 2H), 8.14 (s, 1H), 8.68 (s, 1H), 8.77 (s, 1H), 8.89 (s, 1H).

### Example 25: Preparation of (E)-2-Cyano-3-(3,5-dibromopyridin-2-yl)-N-(1-phenylcycloheptyl)acrylamide (Compound 25)

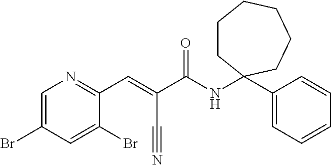 [SMILES: N#C/C(=C\c1ncc(Br)cc1Br)C(=O)NC1(c2ccccc2)CCCCCC1]

Following the procedure for Example 1, but using 3,5-dibromopicolinaldehyde in place of 6-bromo-2-pyridinecarboxaldehyde and using 1-phenylcycloheptylamine hydrochloride in place of 1-phenylcyclopentan-1-amine, the title compound was prepared. ¹H NMR (DMSO-d6): δ 1.51-1.73 (br m, 8H), 2.04 (m, 2H), 2.28 (m, 2H), 7.16 (t, 1H), 7.28 (t, 2H), 7.36 (t, 2H), 8.12 (s, 1H), 8.54 (s, 1H), 8.67 (s, 1H), 8.89 (s, 1H).

### Example 26: Preparation of (E)-2-cyano-3-(3,5-dibromopyridin-2-yl)-N-(1-(m-tolyl)cyclopentyl)acrylamide (Compound 26)

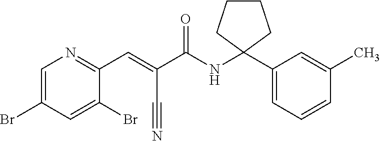 [SMILES: Cc1cccc(C2(NC(=O)/C(C#N)=C/c3ncc(Br)cc3Br)CCCC2)c1]

Following the procedure for Example 1, but using 3,5-dibromopicolinaldehyde in place of 6-bromo-2-pyridinecarboxaldehyde and using 1-(3-methylphenyl)cyclopentan-1-amine in place of 1-phenylcyclopentan-1-amine, the title compound was prepared. ¹HNMR (DMSO-d6): δ 1.75 (m, 4H), 1.97 (m, 2H), 2.28 (s, 3H), 2.42 (m, 2H), 7.00 (m, 1H), 7.13-7.24 (m, 3H), 7.42 (s, 1H), 8.09 (s, 1H), 8.63-8.72 (m, 2H), 8.88 (s, 1H).

### Example 27: Preparation of (E)-N-(1-(4-(tert-butyl)phenyl)cyclopentyl)-2-cyano-3-(3,5-dibromopyridin-2-yl)acrylamide (Compound 27)

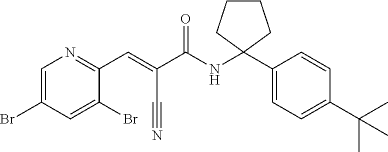 [SMILES: CC(C)(C)c1ccc(C2(NC(=O)/C(C#N)=C/c3ncc(Br)cc3Br)CCCC2)cc1]

Following the procedure for Example 1, but using 3,5-dibromopicolinaldehyde in place of 6-bromo-2-pyridinecarboxaldehyde and using 1-(4-tert-butylphenyl)cyclopentan-1-amine in place of 1-phenylcyclopentan-1-amine, the title compound was prepared. ¹HNMR (DMSO-d6): δ 1.25 (s, 9H), 1.75 (m, 4H), 1.95 (m, 2H), 2.42 (m, 2H), 7.26-7.41 (m, 4H), 8.11 (s, 1H), 8.63-8.72 (m, 2H), 8.88 (s, 1H).

### Example 28: Preparation of (E)-3-(4-bromopyridin-2-yl)-2-cyano-N-(1-phenylcyclopentyl)acrylamide (Compound 28)

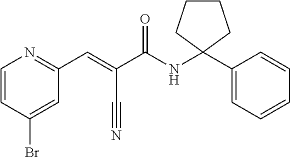 [SMILES: N#C/C(=C\c1cc(Br)ccn1)C(=O)NC1(c2ccccc2)CCCC1]

Following the procedure for Example 1, but using 4-bromopyridine-2-carbaldehyde in place of 6-bromo-2-pyridinecarboxaldehyde, the title compound was prepared. ¹HNMR (DMSO-d6): δ 1.75 (m, 4H), 1.98 (m, 2H), 2.41 (m, 2H), 7.18 (t, 1H), 7.29 (t, 2H), 7.39 (d, 2H), 7.81 (d, 1H), 7.95 (s, 1H), 8.11 (s, 1H), 8.59-8.67 (m, 2H). MS (ES⁺) m/z 396 (M⁺).

### Example 29: Preparation of (E)-3-(5-bromopyridin-2-yl)-2-cyano-N-(1-phenylcyclopentyl)acrylamide (Compound 29)

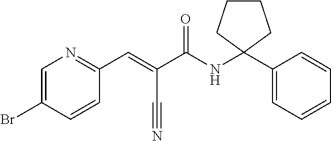 [SMILES: N#C/C(=C\c1ccc(Br)cn1)C(=O)NC1(c2ccccc2)CCCC1]

Following the procedure for Example 1, but using 5-bromopyridine-2-carbaldehyde in place of 6-bromo-2-pyridinecarboxaldehyde, the title compound was prepared. MS (ES⁺) m/z 396 (M⁺).

### Example 30: Preparation of (E)-3-([2,3′-bipyridin]-6-yl)-2-cyano-N-(1-phenylcyclopentyl)acrylamide (Compound 30)

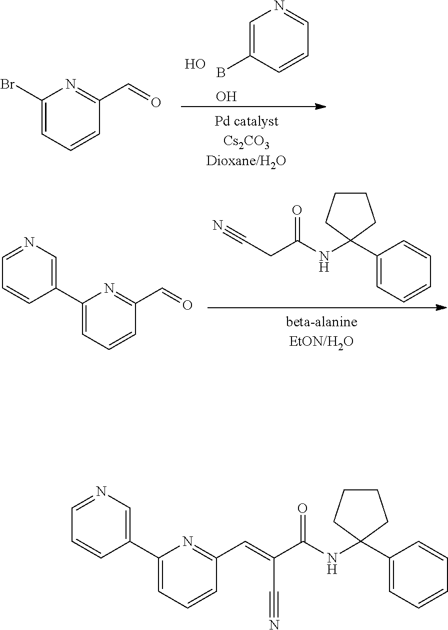

Step 1:

In a sealed vial, a mixture of 6-bromo-2-pyridinecarboxaldehyde (50 mg, 0.27 mmol), 3-pyridinylboronic acid (39 mg, 0.32 mmol), cesium carbonate (264 mg, 0.81 mmol) was suspended in a mixture of 1,4-dioxane (3.6 mL) and di water (0.4 mL). The mixture was purged with nitrogen. To the mixture was then added Pd(dppf)Cl₂ (20 mg, 0.027 mmol). The mixture was then heated at 100° C. under N₂ for 3-4 hours. After completion, the mixture was filtered through Celite, and evaporated to dryness. The residue was extracted with ethyl acetate (5 mL) and water (5 mL) twice. The combined organic layer was washed with brine, dried over sodium sulfate, filtered, and evaporated to dryness. The residue was used for the step 2 without purification.

Step 2:

The residue was re-dissolved in a mixture of ethanol (2 mL) and water (2 mL). To the mixture was then added β-alanine (96 mg, 1.08 mmol), followed by nitrile (31 mg, 0.135 mmol) prepared in situ following the procedure for example 1 step 1. The resulting mixture was then stirred at room temperature overnight and a precipitate formed. The product was then collected via filtration, and rinsed with a solution of 1:1 ethanol:water 3× to afford (E)-3-([2,3′-bipyridin]-6-yl)-2-cyano-N-(1-phenylcyclopentyl)acrylamide (Compound 30) (31 mg, 29%) as a tan solid. ¹HNMR (DMSO-d6): δ 1.77 (m, 4H), 1.98 (m, 2H), 2.41 (m, 2H), 7.20 (t, 1H), 7.29 (t, 2H), 7.40 (d, 2H), 7.79 (d, 1H), 8.05-8.16 (m, 2H), 8.27 (d, 1H), 8.62-8.73 (m, 3H), 9.51 (s, 1H). MS (ES⁺) m/z 395 (M+H)⁺.

### Example 31: Preparation of (E)-ethyl 4-(6-(2-cyano-3-oxo-3-((1-phenylcyclopentyl)amino)prop-1-en-1-yl)pyridin-2-yl)benzoate (Compound 31)

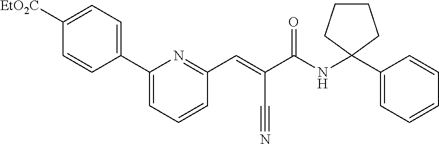 [SMILES: CCOC(=O)c1ccc(-c2cccc(/C=C(\C#N)C(=O)NC3(c4ccccc4)CCCC3)n2)cc1]

Following the procedure for Example 30, using 4-(ethoxycarbonyl)phenylboronic acid in place of 3-pyridinylboronic acid, the title compound was prepared. MS (ES⁺) m/z 466 (M+H)⁺.

### Example 32: Preparation of (E)-ethyl 4′-(1-(2-cyano-3-(3,5-dibromopyridin-2-yl)acrylamido)cyclopentyl)-[1,1′-biphenyl]-4-carboxylate (Compound 32)

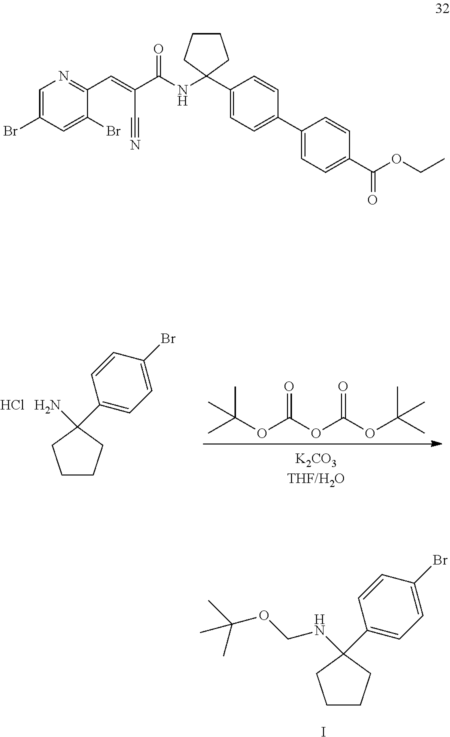

Step 1:

K₂CO₃ (150 mg, 1.08 mmo) in water (1 mL) was added to a stirring solution of 1-(4-bromophenyl)cyclopentan-1-amine hydrochloride (100 mg, 0.36 mmol) and di-tert-butyl dicarbonate (103 mg, 0.47 mmol) in THF (2 mL) at 0° C. The reaction stirred at 0° C. to RT overnight. The next day the mixture diluted with water and extracted with EtOAc. The organic layer was washed with brine, dried over Na₂SO₄, filtered, and concentrated under reduced pressure to afford I as a white solid. The solid was used for step 2 without further purification.

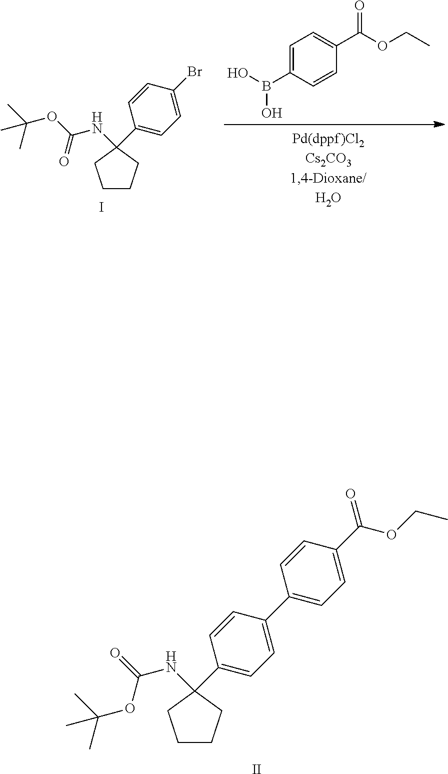

Step 2:

Bromide I (110 mg, 0.32 mmol), 4-ethoxycarbonyl phenylboronic acid (75 mg, 0.39 mmol), Pd(dppf)Cl₂ (24 mg, 0.03 mmol), cesium carbonate (316 mg, 0.97 mmol), and 1,4-dioxane (3.0 mL) in a 40 mL vial was degassed 3 times with nitrogen. Water (0.3 mL) was added to the mixture and the vial sealed. The reaction was heated at 90° C. for 2 hrs. The mixture was filtered through a pad of Celite and washed with minimal EtOAc and water. The filtrate was diluted with water and extracted with EtOAc. The organic layer was washed with brine, dried over Na₂SO₄, filtered, and concentrated under reduced pressure. The crude material was purified by silica gel chromatography 0-25% EtOAc/Hexane to afford II (95 mg, 75%) as a white solid.

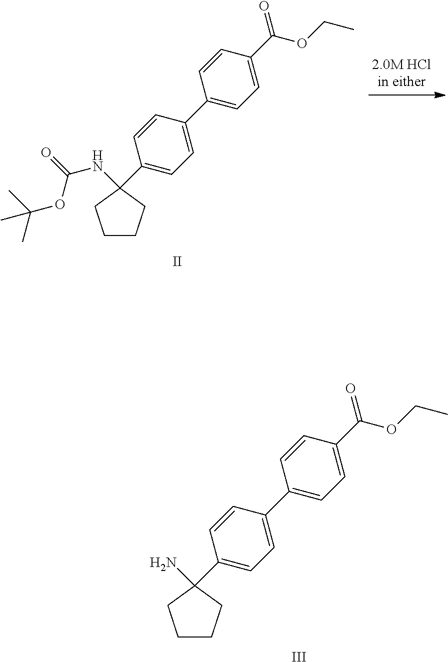

Step 3:

Ester II (55 mg, 0.13 mmol) and 2.0 M HCl in ether (2 mL) in a 4 mL vial was stirred at RT overnight. The next day the mixture was diluted with EtOAc and water. The layers separated and the aqueous layer was basified with sat. NaHCO₃. The mixture extracted with EtOAc. The organic layer was washed with brine, dried over Na₂SO₄, filtered, and concentrated under reduced pressure to afford the amine III (24 mg, 58%) as a white solid. The solid was used for step 4 without further purification.

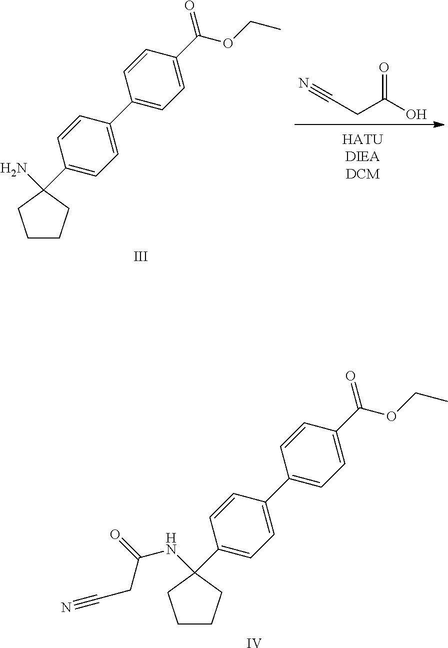

Step 4:

The amine III (24 mg, 0.08 mmol), HATU (32 mg, 0.09 mmol), and DCM (1 mL) in 4 mL vial stirred at RT for 5 min. Cyanoacetic acid (7 mg, 0.08 mmol) and N,N-diisopropyl ethylamine (28 μL, 0.16 mmol) was added to the mixture and stirred at RT for 2 hrs. The reaction was diluted with DCM, washed with water, washed with brine, dried over Na₂SO₄, filtered, and concentrated under reduced pressure to afford the nitrile IV (29 mg, 100%) as an off white solid. The solid was used for step 5 without further purification.

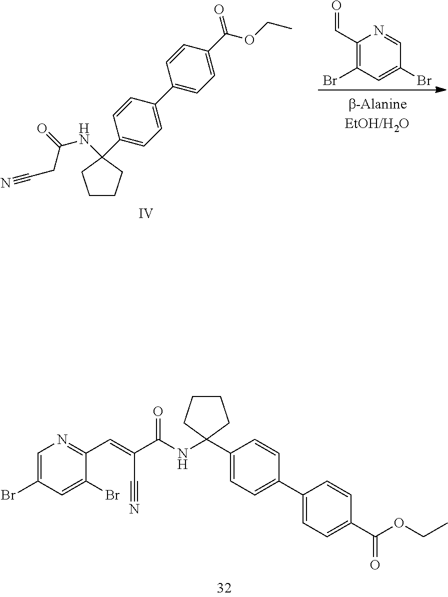

Step 5:

The nitrile IV (29 mg, 0.08 mmol), 3,5-dibromopyridine-2-carboxaldehyde (41 mg, 0.16 mmol), β-alanine (55 mg, 0.62 mmol), and EtOH:water (1:1) (2 mL) in a 4 mL vial was heated at 50° C. overnight. The next day the solids were filtered off and purified by purified by silica gel chromatography 0-50% EtOAc/Hexane to afford (E)-ethyl 4′-(1-(2-cyano-3-(3,5-dibromopyridin-2-yl)acrylamido)cyclopentyl)-[1,1′-biphenyl]-4-carboxylate 32 (17 mg, 35%) as a beige solid. ¹HNMR (DMSO-d6): δ 1.32 (t, 3H), 1.79 (m, 4H), 2.04 (m, 2H), 2.42 (m, 2H), 4.32 (q, 2H), 7.52 (d, 2H), 7.68 (d, 2H), 7.81 (d, 2H), 8.20 (d, 2H), 8.12 (m, 1H), 8.66-8.67 (m, 1H), 8.81 (s, 1H), 8.87-8.88 (m, 1H).

### Example 33: Preparation of (E)-methyl 6-(2-cyano-3-oxo-34(1-phenylcyclopentyl)amino)prop-1-en-1-yl)-[2,3′-bipyridine]-5′-carboxylate (Compound 33)

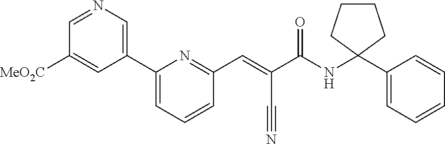 [SMILES: COC(=O)c1cncc(-c2cccc(/C=C(\C#N)C(=O)NC3(c4ccccc4)CCCC3)n2)c1]

Following the procedure for Example 30, using 3-(methoxycarbonyl)pyridine-5-boronic acid, pinacol ester in place of 3-pyridinylboronic acid, the title compound was prepared. MS (ES⁺) m/z 453 (M+H)⁺.

### Example 34: Preparation of (E)-methyl 3-(6-(2-cyano-3-oxo-34(1-phenylcyclopentyl)amino)prop-1-en-1-yl)pyridin-2-yl)benzoate (Compound 34)

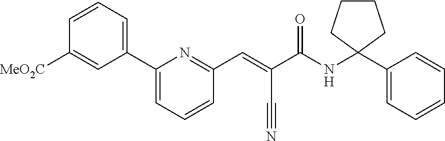 [SMILES: COC(=O)c1cccc(-c2cccc(/C=C(\C#N)C(=O)NC3(c4ccccc4)CCCC3)n2)c1]

Following the procedure for Example 30, using 3-methoxycarbonylphenyl boronic acid, pinacol ester in place of 3-pyridinylboronic acid, the title compound was prepared. MS (ES⁺) m/z 452 (M+H)⁺.

### Example 35: Preparation of (E)-3-(6-bromopyridin-2-yl)-2-cyano-N-methyl-N-(1-phenylcyclopentyl)acrylamide (Compound 2-82)

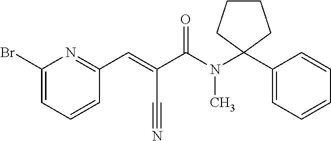 [SMILES: CN(C(=O)/C(C#N)=C/c1cccc(Br)n1)C1(c2ccccc2)CCCC1]

Following the procedure for Example 1, but using 1-methyl 1-phenylcyclopentan-1-amine prepared in situ in place of 1-phenylcyclopentan-1-amine, the title compound was prepared. MS (ES⁺) m/z 432 (M+Na)⁺.

### Example 36: Preparation of (E)-2-cyano-3-(3,5-dibromopyridin-2-yl)-N-(1-phenylcyclopentyl)-N-(pyridin-3-ylmethyl)acrylamide (Compound 35)

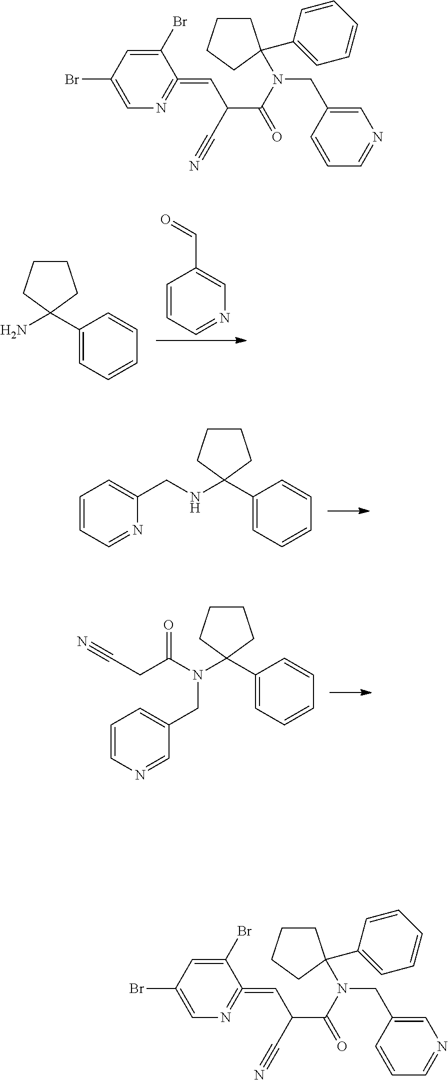

Step 1:

In a 40 ml Vial, a mixture of 1-phenylcyclopentan-1-amine (100 mg, 0.62 mmol) and pyridine-3-carbaldehyde (66 mg, 0.62 mmol) was suspended in EtOH (4 mL). To the mixture was added sodium cyanoborohydride (51 mg, 0.81 mmol) and HOAc (30 μL). The mixture was purged with N₂, and heated at 60° C. for 4-5 hours. The mixture was then extracted with EtOAc and water. The organic layer was washed with brine, dried over Na₂SO₄, filtered and evaporated to dryness.

Step 2 and Step 3:

Following the procedure for Example 1, but using 3,5-dibromopicolinaldehyde in place of 6-bromo-2-pyridinecarboxaldehyde, the title compound (36 mg) was prepared as an off-white solid. MS (ES⁺) m/z 567 (M+H)⁺.

### Example 37: Preparation of (E)-2-(4-(6-(2-cyano-3-oxo-34(1-phenylcyclopentyl)amino)prop-1-en-1-yl)pyridin-2-yl)phenoxy)acetic acid (Compound 36)

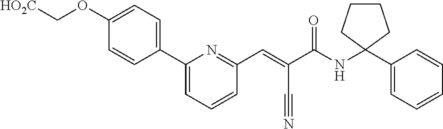 [SMILES: N#C/C(=C\c1cccc(-c2ccc(OCC(=O)O)cc2)n1)C(=O)NC1(c2ccccc2)CCCC1]

Following the procedure for Example 30, using 2-[4-(dihydroxyboranyl)phenoxy]acetic acid in place of 3-pyridinylboronic acid, the title compound was prepared. MS (ES⁺) m/z 468 (M+H)⁺.

### Example 38: Preparation of (E)-methyl 5-(4-(1-(2-cyano-3-(3,5-dibromopyridin-2-yl)acrylamido) cyclopentyl)phenyl)picolinate (Compound 37)

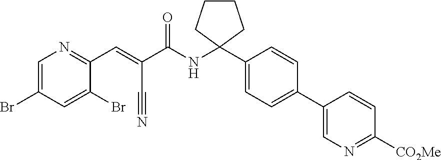 [SMILES: COC(=O)c1ccc(-c2ccc(C3(NC(=O)/C(C#N)=C/c4ncc(Br)cc4Br)CCCC3)cc2)cn1]

Following the procedure for Example 32, using 6-(methoxycarbonyl)pyridine-3-boronic acid in place of 4-ethoxycarbonylphenylboronic acid, the title compound was prepared. MS (ES⁺) m/z 611 (M+H)⁺.

### Example 39: Preparation of (E)-methyl 4′-(1-(2-cyano-3-(3,5-dibromopyridin-2-yl)acrylamido) cyclopentyl)-[1,1′-biphenyl]-3-carboxylate (Compound 38)

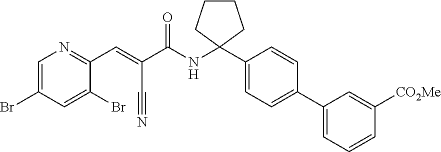 [SMILES: COC(=O)c1cccc(-c2ccc(C3(NC(=O)/C(C#N)=C/c4ncc(Br)cc4Br)CCCC3)cc2)c1]

Following the procedure for Example 32, using 3-methoxycarbonylphenylboronic acid, pinacol ester in place of 4-ethoxycarbonylphenylboronic acid, the title compound was prepared. MS (ES⁺) m/z 634 (M+Na)⁺.

### Example 40: Preparation of (E)-2-cyano-3-(3,5-dibromopyridin-2-yl)-N-(1-(4-(pyridin-3-yl)phenyl) cyclopentyl)acrylamide (Compound 39)

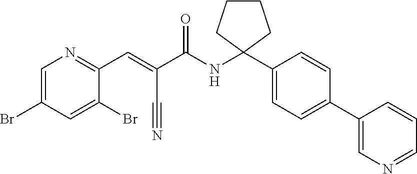 [SMILES: N#C/C(=C\c1ncc(Br)cc1Br)C(=O)NC1(c2ccc(-c3cccnc3)cc2)CCCC1]

Following the procedure for Example 32, using pyridine-3-boronic acid in place of 4-ethoxycarbonylphenylboronic acid, the title compound was prepared. MS (ES⁺) m/z 553 (M+H)⁺.

### Example 41: (E)-2-(4-(6-(2-cyano-3-oxo-34(1-phenylcyclopentyl)amino)prop-1-en-1-yl)pyridin-2-yl)phenyl)acetic acid (Compound 40)

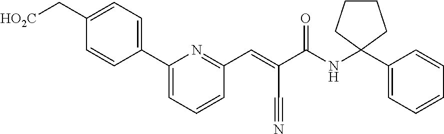 [SMILES: N#C/C(=C\c1cccc(-c2ccc(CC(=O)O)cc2)n1)C(=O)NC1(c2ccccc2)CCCC1]

Following the procedure for Example 30, using 2-[4-(dihydroxyboranyl)phenyl]acetic acidin place of 3-pyridinylboronic acid, the title compound was prepared. MS (ES⁺) m/z 452 (M+H)⁺.

### Example 42: Preparation of (E)-methyl 4-(1-(3-(6-bromopyridin-2-yl)-2-cyanoacrylamido) cyclopentyl)benzoate (Compound 41)

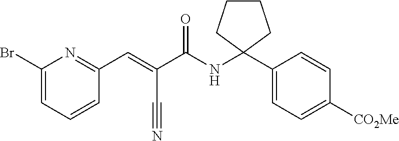 [SMILES: COC(=O)c1ccc(C2(NC(=O)/C(C#N)=C/c3cccc(Br)n3)CCCC2)cc1]

Following the procedure for Example 1, but using methyl 4-(1-aminocyclopentyl)benzoate in place of 1-phenylcyclopentan-1-amine, the title compound was prepared. MS (ES+) m/z 454 (M+H)⁺.

### Example 43: Preparation of (E)-methyl 3-(6-((E)-2-cyano-3-oxo-3-((1-phenylcyclopentyl)amino)prop-1-en-1-yl)pyridin-2-yl)acrylate (Compound 42)

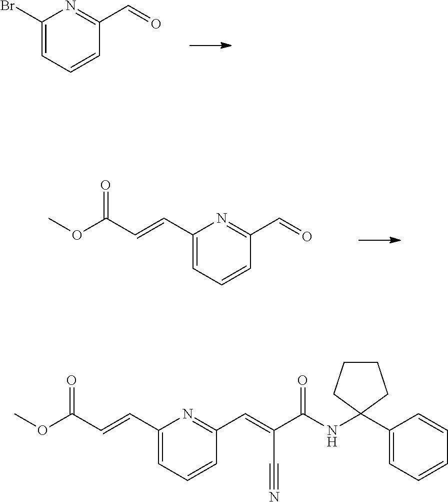 [SMILES: O=Cc1cccc(Br)n1 ; COC(=O)/C=C/c1cccc(C=O)n1 ; COC(=O)/C=C/c1cccc(/C=C(\C#N)C(=O)NC2(c3ccccc3)CCCC2)n1]

Step 1:

In a sealed vial, a mixture of 6-bromo-2-pyridinecarboxaldehyde (50 mg, 0.27 mmol), methyl acrylate (29 μL, 0.32 mmol), Pd(OAc)2 (6 mg, 0.03 mmol), triphenylphosphine (14 mg, 0.054 mmol) and Et₃N (75 μL, 0.54 mmol) was suspended in 1,4-dioxane (5 mL). The mixture was then heated at 60° C. under N₂ for overnight. After completion, the mixture was filtered through Celite, and evaporated to dryness. The residue was extracted with EtOAc (5 mL) and water (5 mL). The combined organic layer was washed with brine, dried over Na₂SO₄, filtered, and evaporated to dryness. The residue was purified on silica gel chromatography 0-100% EtOAc/Hexane to afford 10 mg desired aldehyde.

Step 2:

Following the procedure for example 1, but using product from step 1 in place of 6-bromo-2-pyridinecarboxaldehyde, the title compound (Compound 42) was prepared. MS (ES⁺) m/z 402 (M+H)⁺.

### Example 44: Preparation of (E)-3-(6-(3-(1H-pyrazol-1-yl)prop-1-yn-1-yl)pyridin-2-yl)-2-cyano-N-(1-phenylcyclopentyl)acrylamide (Compound 43)

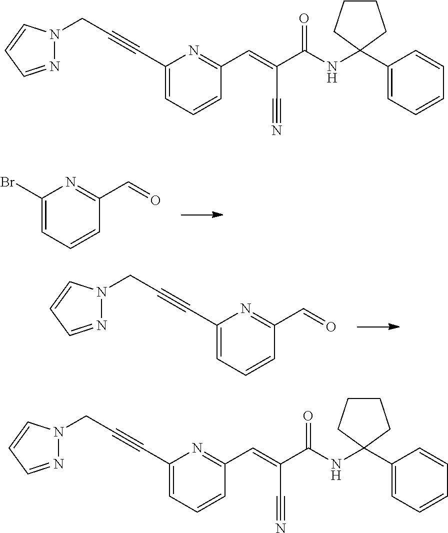

Step 1:

In a sealed vial, a mixture of 6-bromo-2-pyridinecarboxaldehyde (100 mg, 0.54 mmol), 1-(prop-2-yn-1-yl)-1H-pyrazole (74 mg, 0.70 mmol), Pd(PPh₃)₄ (62 mg, 0.054 mmol), CuI (10 mg, 0.054 mmol) and trimethylamine (152 μL, 1.08 mmol) was suspended in dry DMF (4 mL). The mixture was then heated at 60° C. under N₂ for 3˜4 hours. After completion, the mixture was filtered through Celite, and evaporated to dryness. The residue was extracted with EtOAc (5 mL) and water (5 mL). The combined organic layer was washed with brine, dried over Na₂SO₄, filtered, and evaporated to dryness. The residue was purified on silica gel chromatography 0-100% EtOAc/Hexane to afford 37 mg of the desired aldehyde.

Step 2:

Following the procedure for example 1, but using product from step 1 in place of 6-bromo-2-pyridinecarboxaldehyde, the title compound (Compound 43) was prepared. MS (ES⁺) m/z 422 (M+H)⁺.

### Example 45: Preparation of (E)-N-(1-(4-(3-(1H-pyrazol-1-yl)prop-1-yn-1-yl)phenyl)cyclopentyl)-2-cyano-3-(3,5-dibromopyridin-2-yl)-N-methylacrylamide (Compound 44)

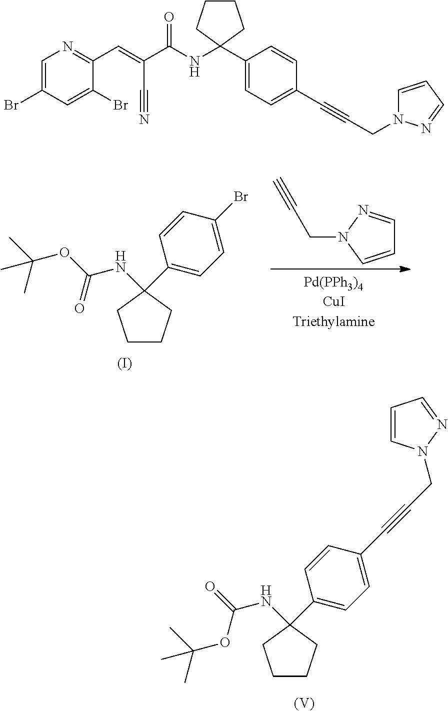

Step 1:

Example 32 Intermediate (I) (100 mg, 0.29 mmol), 1-(prop-2-yn-1-yl)-1H-pyrazole (41 mg, 0.38 mmol), Pd(PPh₃)₄ (34 mg, 0.03 mmol), CuI (6 mg, 0.03 mmol), Et₃N (82 μL, 0.59 mmol), and DMF (1.5 mL) in 40 mL vial was degassed with N₂. The reaction heated at 60° C. overnight. The mixture diluted with water and EtOAc and filtered through pad of celite. The filtrate was diluted with water and extracted with EtOAc (2×). The organic layers were combined, washed with brine, dried over Na₂SO₄, filtered, and concentrated under reduced pressure. The crude material was purified by silica gel chromatography 0-30% EtOAc/Hexane to afford Intermediate (V) as a yellow oil (23 mg, 22%) MS (ES⁺) m/z 366 (M+H)⁺.

Step 2-4:

Following produce from Example 32 Step 3-5 using the above Intermediate (V) in place of Example 32 Intermediate (II), the title compound was prepared. MS (ES⁺) m/z 580 (M+H)⁺.

### Example 46: Preparation of (E)-2-cyano-3-(64(1-hydroxycyclopentyl)ethynyl)pyridin-2-yl)-N-(1-phenylcyclopentyl)acrylamide (Compound 45)

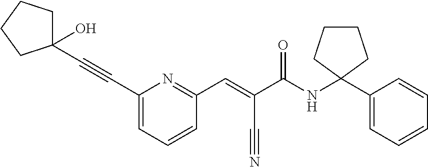 [SMILES: N#C/C(=C\c1cccc(C#CC2(O)CCCC2)n1)C(=O)NC1(c2ccccc2)CCCC1]

Following the procedure for Example 44, but using 1-ethynylcyclopentanol in place of 1-(prop-2-yn-1-yl)-1H-pyrazole, the title compound (compound 45) was prepared. MS (ES+) m/z 426 (M+H)⁺.

### Example 47: Preparation of (E)-3-(3-bromopyridin-2-yl)-2-cyano-N-(1-phenylcyclopentyl)acrylamide (Compound 46)

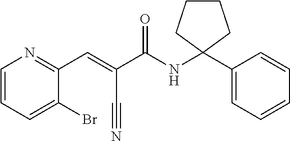 [SMILES: N#C/C(=C\c1ncccc1Br)C(=O)NC1(c2ccccc2)CCCC1]

Following the procedure for Example 1, but using 3-bromo-2-pyridinecarboxaldehyde in place of 6-bromo-2-pyridinecarboxaldehyde, the title compound (Compound 46) was prepared. MS (ES+) m/z 397 (M+H)⁺.

### Example 48: Preparation of (E)-2-cyano-3-(2′-ethoxy-5′-(trifluoromethyl)-[2,3′-bipyridin]-6-yl)-N-(1-phenylcyclopentyl)acrylamide (Compound 47)

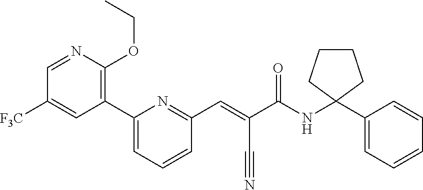 [SMILES: CCOc1ncc(C(F)(F)F)cc1-c1cccc(/C=C(\C#N)C(=O)NC2(c3ccccc3)CCCC2)n1]

Following the procedure for Example 30, using 2-ethoxy-5-(trifluoromethyl)pyridine-3-boronic acid in place of 3-pyridinylboronic acid, the title compound was prepared. MS (ES⁺) m/z 507 (M+H)⁺.

### Example 49: Preparation of (E)-methyl 3-(1-(3-(6-bromopyridin-2-yl)-2-cyanoacrylamido) cyclopentyl)benzoate (Compound 48)

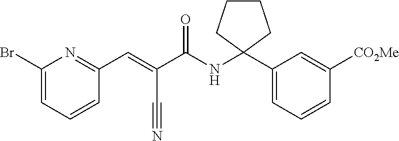 [SMILES: COC(=O)c1cccc(C2(NC(=O)/C(C#N)=C/c3cccc(Br)n3)CCCC2)c1]

Following the procedure for Example 1, but using methyl 3-(1-aminocyclopentyl)benzoate in place of 1-phenylcyclopentan-1-amine, the title compound (Compound 3-7) was prepared. MS (ES+) m/z 454 (M+H)⁺.

### Example 50: Preparation of (E)-tert-butyl 6′-chloro-6-(2-cyano-3-oxo-34(1-phenylcyclopentyl) amino)prop-1-en-1-yl)[2,3′-bipyridine]-5′-carboxylate (Compound 49)

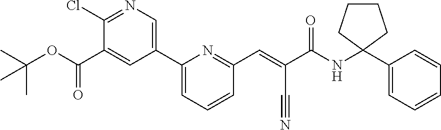 [SMILES: CC(C)(C)OC(=O)c1cc(-c2cccc(/C=C(\C#N)C(=O)NC3(c4ccccc4)CCCC3)n2)cnc1Cl]

Following the procedure for Example 30, using 3-t-butoxycarbonyl-2-chloropyridine-5-boronic acid, pinacol ester in place of 3-pyridinylboronic acid, the title compound was prepared. MS (ES⁺) m/z 529 (M+H)⁺.

### Example 51: Preparation of (E)-2-cyano-3-(3,5-dibromopyridin-2-yl)-N-(1-(4-(3-morpholinoprop-1-yn-1-yl)phenyl)cyclopentyl)acrylamide (Compound 50)

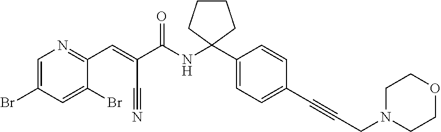 [SMILES: N#C/C(=C\c1ncc(Br)cc1Br)C(=O)NC1(c2ccc(C#CCN3CCOCC3)cc2)CCCC1]

Following the procedure for Example 45, using 1-(N-morpholino)-2-propyne in place of 1-(prop-2-yn-1-yl)-1H-pyrazole, the title compound was prepared. MS (ES⁺) m/z 599 (M+H)⁺.

### Example 52: (E)-2-cyano-3-(3,5-dibromopyridin-2-yl)-N-(1-(4-(6-morpholinopyridin-3-yl)phenyl)cyclopentyl)acrylamide (Compound 51)

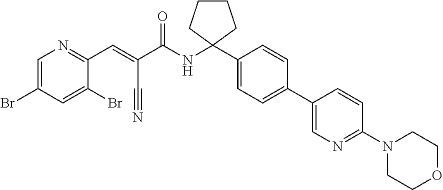 [SMILES: N#C/C(=C\c1ncc(Br)cc1Br)C(=O)NC1(c2ccc(-c3ccc(N4CCOCC4)nc3)cc2)CCCC1]

Following the procedure for Example 32, using 4-(5-(4,4,5,5-tetramethyl-1,3,2-dioxaborolan-2-yl)pyridine-2-yl)morpholine in place of 4-ethoxycarbonylphenylboronic acid, the title compound was prepared. MS (ES⁺) m/z 638 (M+H)⁺.

### Example 53: Preparation of (E)-tert-butyl 3-(1-(2-cyano-3-(3,5-dibromopyridin-2-yl)acrylamido) cyclopentyl)benzoate (Compound 52)

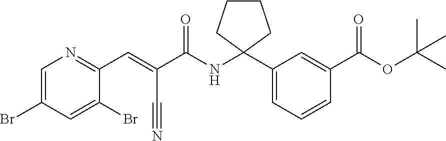 [SMILES: CC(C)(C)OC(=O)c1cccc(C2(NC(=O)/C(C#N)=C/c3ncc(Br)cc3Br)CCCC2)c1]

Following the procedure for Example 1, but using methyl tert-butyl 3-(1-aminocyclopentyl)benzoate in place of 1-phenylcyclopentan-1-amine, the title compound (Compound 53) was prepared. MS (ES+) m/z 574 (M+H)⁺.

### Example 54: Preparation of (E)-2-cyano-3-(3,5-dibromopyridin-2-yl)-N-(1-(4-(2-methoxypyridin-3-yl)phenyl)cyclopentyl)acrylamide (Compound 53)

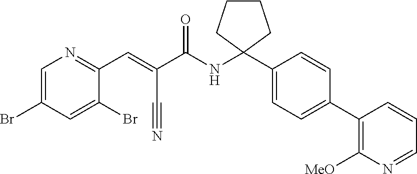 [SMILES: COc1ncccc1-c1ccc(C2(NC(=O)/C(C#N)=C/c3ncc(Br)cc3Br)CCCC2)cc1]

Following the procedure for Example 32, but using 2-methoxy-3-pyridylboronic acid in place of 4-ethoxycarbonylphenylboronic acid, the title compound was prepared. MS (ES⁺) m/z 581 (M+H)⁺.

### Example 55: Preparation of (E)-2-cyano-3-(3,5-dibromopyridin-2-yl)-N-(1-(4-(2-ethoxy-5-(trifluoromethyl)pyridin-3-yl)phenyl)cyclopentyl)acrylamide (Compound 54)

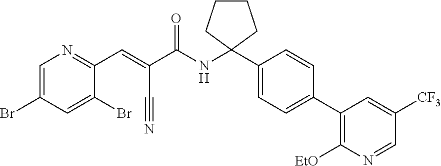 [SMILES: CCOc1ncc(C(F)(F)F)cc1-c1ccc(C2(NC(=O)/C(C#N)=C/c3ncc(Br)cc3Br)CCCC2)cc1]

Following the procedure for Example 32, using [2-ethoxy-5-(trifluoromethyl)pyridin-3-yl]boronic acidin place of 4-ethoxycarbonylphenylboronic acid, the title compound was prepared. MS (ES⁺) m/z 663 (M+H)⁺.

### Example 56: Preparation of (E)-2-cyano-3-(6-(3-morpholinoprop-1-yn-1-yl)pyridin-2-yl)-N-(1-phenylcyclopentyl)acrylamide (Compound 55)

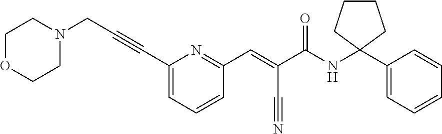 [SMILES: N#C/C(=C\c1cccc(C#CCN2CCOCC2)n1)C(=O)NC1(c2ccccc2)CCCC1]

Following the procedure for Example 44, but using 4-(Prop-2-yn-1-yl)morpholine in place of 1-(prop-2-yn-1-yl)-1H-pyrazole, the title compound (compound 55) was prepared. MS (ES+) m/z 441 (M+H)⁺.

### Example 57: Preparation of (E)-tert-butyl 3-(6-(2-cyano-3-oxo-3-((1-phenylcyclopentyl)amino)prop-1-en-1-yl)pyridin-2-yl)benzoate (Compound 56)

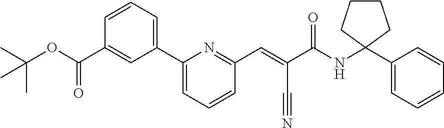 [SMILES: CC(C)(C)OC(=O)c1cccc(-c2cccc(/C=C(\C#N)C(=O)NC3(c4ccccc4)CCCC3)n2)c1]

Following the procedure for Example 30, using tert-Butyl 3-(4,4,5,5-tetramethyl-1,3,2-dioxaborolan-2-yl)benzoate in place of 3-pyridinylboronic acid, the title compound was prepared. MS (ES⁺) m/z 494 (M+H)⁺.

### Example 58: Preparation of (E)-2-cyano-3-(6′-morpholino-[2,3′-bipyridin]-6-yl)-N-(1-phenylcyclopentyl)acrylamide (Compound 57)

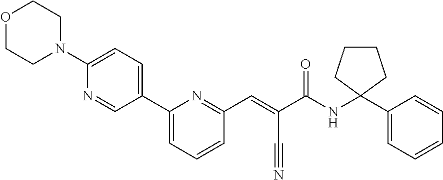 [SMILES: N#C/C(=C\c1cccc(-c2ccc(N3CCOCC3)nc2)n1)C(=O)NC1(c2ccccc2)CCCC1]

Following the procedure for Example 30, using 2-(4-morpholino)pyridine-5-boronic acid pinacol ester in place of 3-pyridinylboronic acid, the title compound was prepared. MS (ES⁺) m/z 480 (M+H)⁺.

### Example 59: Preparation of (E)-N-(1-(3-bromophenyl)cyclopentyl)-3-(6-bromopyridin-2-yl)-2-cyanoacrylamide (Compound 58)

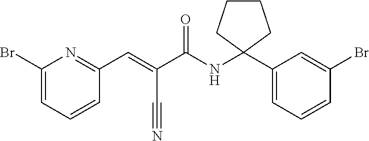 [SMILES: N#C/C(=C\c1cccc(Br)n1)C(=O)NC1(c2cccc(Br)c2)CCCC1]

Following the procedure for Example 1, but using 1-(3-bromophenyl)cyclopentan-1-amine in place of 1-phenylcyclopentan-1-amine, the title compound) was prepared. MS (ES+) m/z 474 (M+H)⁺.

### Example 60: Preparation of (E)-N-(1-(4-(1H-pyrrolo[2,3-b]pyridin-5-yl)phenyl)cyclopentyl)-2-cyano-3-(3,5-dibromopyridin-2-yl)acrylamide (Compound 59)

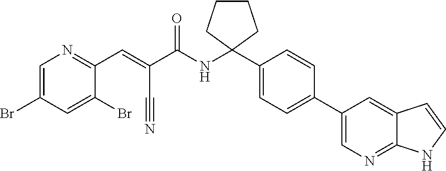 [SMILES: N#C/C(=C\c1ncc(Br)cc1Br)C(=O)NC1(c2ccc(-c3cnc4[nH]ccc4c3)cc2)CCCC1]

Following the procedure for Example 32, using 5-(4,4,5,5-tetramethyl-1,3,2-dioxaborolan-2-yl)-1H-pyrrolo[2,3-b]pyridine in place of 4-ethoxycarbonylphenylboronic acid, the title compound was prepared. MS (ES⁺) m/z 592 (M+H)⁺.

### Example 61: Preparation of (E)-ethyl 3-(6-(2-cyano-3-oxo-34(1-phenylcyclopentyl)amino)prop-1-en-1-yl)pyridin-2-yl)-2-fluorobenzoate (Compound 60)

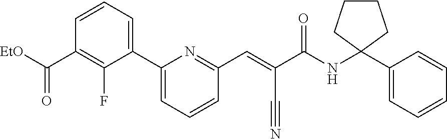 [SMILES: CCOC(=O)c1cccc(-c2cccc(/C=C(\C#N)C(=O)NC3(c4ccccc4)CCCC3)n2)c1F]

Following the procedure for Example 30, using 2-fluoro-3-(ethoxycarbonyl)phenylboronic acid in place of 3-pyridinylboronic acid, the title compound was prepared. MS (ES⁺) m/z 484 (M+H)⁺.

### Example 62: Preparation of (E)-3-(6-(1H-pyrrolo[2,3-b]pyridin-5-yl)pyridin-2-yl)-2-cyano-N-(1-phenylcyclopentyl)acrylamide (Compound 61)

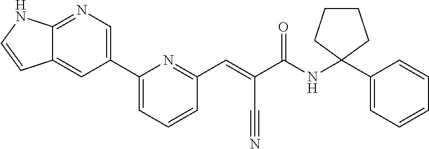 [SMILES: N#C/C(=C\c1cccc(-c2cnc3[nH]ccc3c2)n1)C(=O)NC1(c2ccccc2)CCCC1]

Following the procedure for Example 30, using 5-(4,4,5,5-tetramethyl-1,3,2-dioxaborolan-2-yl)-1H-pyrrolo[2,3-b]pyridine in place of 3-pyridinylboronic acid, the title compound was prepared. MS (ES⁺) m/z 434 (M+H)⁺.

### Example 63: Preparation of (E)-2-cyano-3-(6-(1-methyl-1H-pyrazol-4-yl)pyridin-2-yl)-N-(1-phenylcyclopentyl)acrylamide (Compound 62)

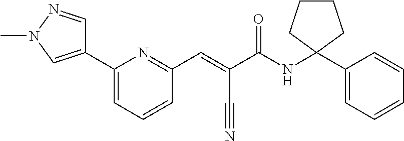 [SMILES: Cn1cc(-c2cccc(/C=C(\C#N)C(=O)NC3(c4ccccc4)CCCC3)n2)cn1]

Following the procedure for Example 30, using (1-methyl-1H-pyrazol-4-yl)boronic acid in place of 3-pyridinylboronic acid, the title compound was prepared. MS (ES⁺) m/z 398 (M+H)⁺.

### Example 64: Preparation of (E)-2-cyano-3-(3,5-dibromopyridin-2-yl)-N-(1-(4-(prop-1-en-2-yl) phenyl)cyclopentyl)acrylamide (Compound 63)

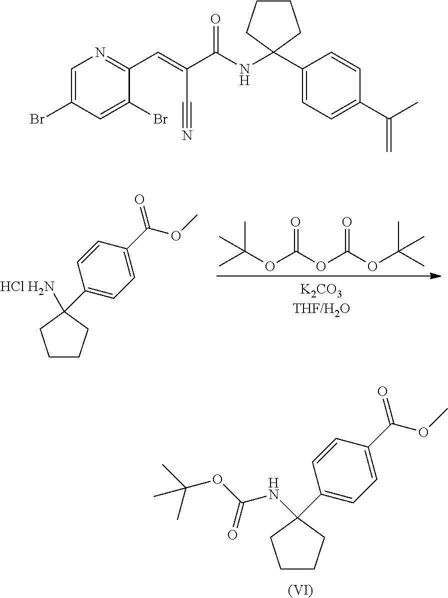

Step 1:

Following produce from Example 32 Step 1 using methyl 3-(1-aminocyclopentyl)benzoate in place of 1-(4-bromophenyl)cyclopentan-1-amine hydrochloride, Intermediate (VI) was prepared. MS (ES⁺) m/z 203 (M-NHBoc)⁺.

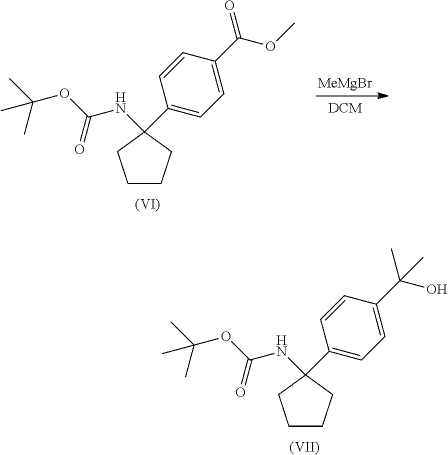

Step 2:

Intermediate (VI) (50 mg, 016 mmol) and DCM (2 mL) in 40 mL vial was degassed with N₂. The vial was cooled in brine water bath. Methylmagnesium bromide (1.4 M in THF:Toluene, 358 μL, 0.50 mmol) was added dropwise. The reaction stirred at −10° C. to RT over 4 hrs. The mixture quenched with sat. NH₄Cl and extracted with DCM (3×). The organic layers were combined, washed with brine, dried over Na₂SO₄, filtered, and concentrated under reduced pressure. The crude material was purified by silica gel chromatography 0-50% EtOAc/Hexane to afford Intermediate (VII) (36 mg, 70%) as a white solid. MS (ES⁺) m/z 342 (M+Na)⁺.

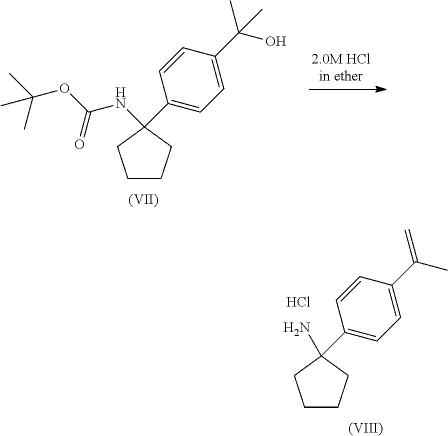

Step 3:

Intermediate (VII) (36 mg, 0.11 mmol) and 2.0 M hydrochloric acid in ether (1.0 mL) in 4 mL vial was stirred overnight. The solvent was removed to afford Intermediate (VIII) as a clear oil (28 mg).

Step 4-5:

Following produce from Example 32 Step 4-5 using the above Intermediate (VIII) in place of Example 32 Intermediate (II), the title compound was prepared. ¹H NMR (DMSO-d6): δ 1.83-1.70 (m, 4H), 2.04-1.94 (m, 2H), 2.08 (s, 3H), 2.48-2.37 (m, 2H), 5.04 (s, 1H), 5.37 (s, 1H), 7.35 (d, 2H), 7.42 (d, 2H), 8.10 (s, 1H), 8.66 (s, 1H), 8.75 (s, 1H), 8.87 (s, 1H).

### Example 65: Preparation of (E)-2-cyano-N-(1-phenylcyclopentyl)-3-(6′-(trifluoromethyl)-[2,3′-bipyridin]-6-yl)acrylamide (Compound 64)

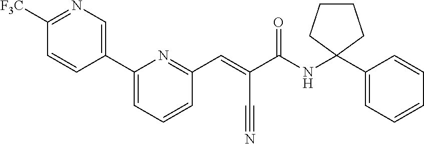 [SMILES: N#C/C(=C\c1cccc(-c2ccc(C(F)(F)F)nc2)n1)C(=O)NC1(c2ccccc2)CCCC1]

Following the procedure for Example 30, using 2-(Trifluoromethyl)pyridin-5-ylboronic acid in place of 3-pyridinylboronic acid, the title compound was prepared. MS (ES⁺) m/z 463 (M+H)⁺.

### Example 66: Preparation of (E)-2-cyano-N-(1-phenylcyclopentyl)-3-(6′-(piperidin-1-yl)-[2,3′-bipyridin]-6-yl)acrylamide (Compound 65)

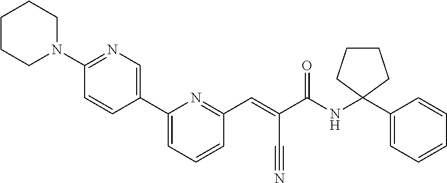 [SMILES: N#C/C(=C\c1cccc(-c2ccc(N3CCCCC3)nc2)n1)C(=O)NC1(c2ccccc2)CCCC1]

Following the procedure for Example 30, using 6-(Piperidin-1-yl)pyridin-3-ylboronic acid in place of 3-pyridinylboronic acid, the title compound was prepared. MS (ES⁺) m/z 478 (M+H)⁺.

### Example 67: Preparation of (E)-4-(1-(2-cyano-3-(3,5-dibromopyridin-2-yl)acrylamido)cyclopentyl)-N,N-dimethylbenzamide (Compound 66)

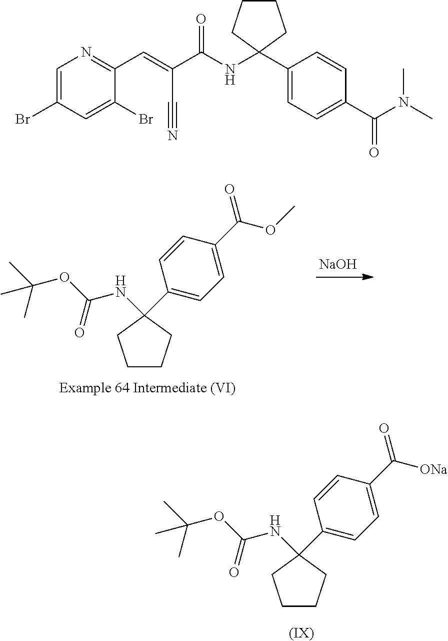

Step 1:

Example 64 Intermediate (VI) (100 mg, 0.31 mmol), sodium hydroxide (1.0 M aq soluntion, 344 μL, 0.34 mmol), and THF:MeOH (3:1) (2 mL) in 4 mL vial was stirred overnight. The next day the reaction heat to 50° C. for 6 hrs. The solvent was removed to afford Intermediate (IX) as a white solid. MS (ES⁺) m/z 189 (M-NHBoc)⁺.

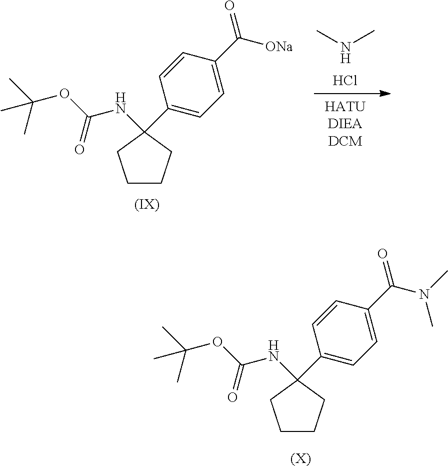

Step 2:

Intermediate (IX) (59 mg, 0.18 mmol), HATU (75 mg, 0.20 mmol), and DCM (2 mL) in 4 mL vial. N,N-diisopropylethylamine (126 μL, 0.72 mmol) and dimethylamine hydrochloride (15 mg, 0.18 mmol) was added to the mixture and stirred at RT overnight. The reaction was washed with water, washed with brine, dried over Na₂SO₄, filtered, and concentrated under reduced pressure. The crude material was purified by silica gel chromatography 0-100% EtOAc/Hexane to afford Intermediate (X) (46 mg, 76%) as white foam. MS (ES⁺) m/z 355 (M+Na)⁺

Step 3-5:

Following produce from Example 32 Step 3-5 using the above Intermediate (X) in place of Example 32 Intermediate (II), the title compound was prepared. MS (ES⁺) m/z 547 (M+H)⁺.

### Example 68: Preparation of (E)-2-cyano-3-(6-cyclopropylpyridin-2-yl)-N-(1-(4-(pyridin-3-yl)phenyl) cyclopentyl)acrylamide (Compound 67)

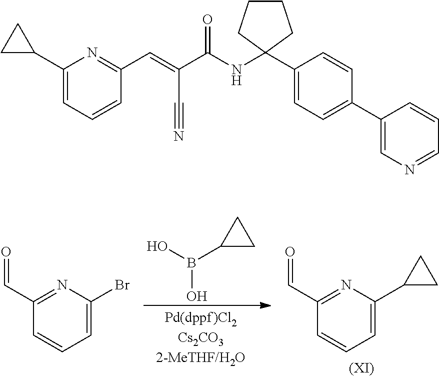

Step 1:

6-Bromopyridine-2-carbaldehyde (1.0 g, 5.38 mmol), cyclopropylboronic acid (693 mg, 8.06 mmol), Pd(dppf)Cl₂ (393 mg, 0.54 mmol), cesium carbonate (5.25 g, 16.1 mmol), 2-methyltetrahydrofuran (8.0 mL), and water (2.0 mL) in a 40 mL vial was degassed 3 times with nitrogen. The reaction was heated at 90° C. overnight. The mixture filtered through a pad of Celite and washed with EtOAc. The filtrate diluted with EtOAc, washed with water, washed with brine, filtered through pad of celite, dried over Na₂SO₄, filtered, and concentrated under reduced pressure. The crude material was purified by silica gel chromatography 0-5% EtOAc/Hexane to afford the Intermediate (XI) (190 mg, 24%) as a yellow liquid. ¹HNMR (DMSO-d6): δ 1.00 (m, 4H), 2.23-2.19 (m, 1H), 7.67-7.56 (m, 2H), 7.88-7.84 (m, 1H), 9.85 (s, 1H).

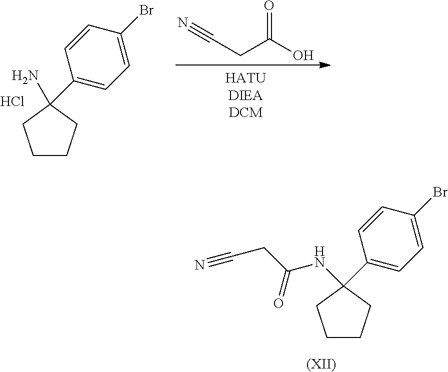

Step 2:

Cyanoacetic acid (1.54 g, 18.1 mmol), HATU (7.2 g, 19.0 mmol), and DCM (90 mL) in 250 mL round bottom flask was cooled in brine water bath. N,N-diisopropylethylamine (3.15 mL, 18.1 mmol) was added and stirred −10° C. for 10 min. 1-(4-bromophenyl)cyclopentan-1-amine hydrochloride (5.0 g, 18.1 mmol) and N,N-diisopropyl ethylamine (6.3 mL, 36.2 mmol) was added to the mixture and stirred at −10° C. to RT overnight. The reaction was washed with water, washed with brine, dried over Na₂SO₄, filtered, and concentrated under reduced pressure. The crude material was flashed on a silica gel chromatography 0-10% EtOAc/DCM. The product fractions were combined and concentrated under reduced pressure. Solids crashed out and filtered to afford Intermediate (XII) (4.5 g, 81%) as a white solid. ¹HNMR (DMSO-d6): δ 1.86-1.66 (m, 6H), 2.24-2.19 (m, 2H), 3.62 (s, 2H), 7.28 (d, 1H), 7.44 (d, 2H), 8.45 (s, 1H).

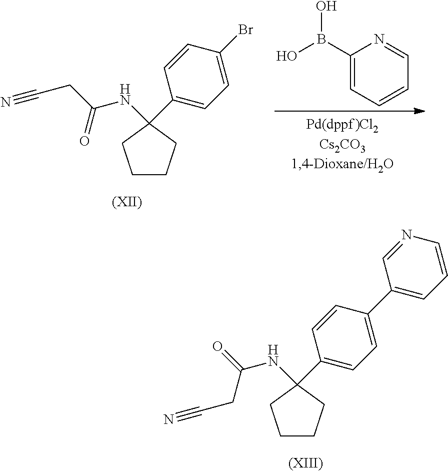

Step 3:

Intermediate (XII) (1.0 g, 3.26 mmol), pyridine-3-boronic acid (480 mg, 3.91 mmol), Pd(dppf)Cl₂ (238 mg, 0.33 mmol), cesium carbonate (3.18 g, 9.77 mmol), 1,4-dioxane (10.0 mL), and water (2.5 mL) in a 40 mL vial was degassed 3 times with nitrogen. The reaction was heated at 80° C. for 4 hrs. The mixture was diluted with water and filtered through a pad of Celite. The filtrate extracted with EtOAc (2×). The organic layers were combined, washed with brine, dried over Na₂SO₄, filtered, and concentrated under reduced pressure. The crude material was purified by silica gel chromatography 0-100% EtOAc/Hexane to afford Intermediate (XIII) (678 mg, 68%) as an off white solid. MS (ES⁺) m/z 306 (M+H)⁺.

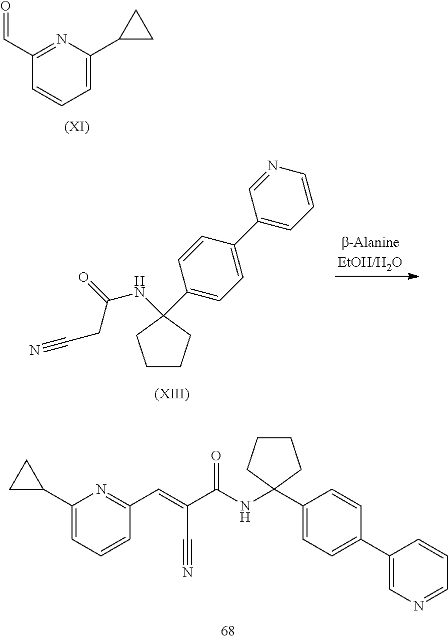

Step 4:

Intermediate (XI) (31 mg, 0.21 mmol), Intermediate (XIII) (43 mg, 0.14 mmol), β-alanine (100 mg, 1.13 mmol), and EtOH:water (1:1) (2 mL) in 4 mL vial. The reaction stirred at RT for 5 days. The mixture was diluted with water and extracted with DCM (3×). The organic layers were combined, washed with brine, dried over Na₂SO₄, filtered, and concentrated under reduced pressure. The crude material was purified by silica gel chromatography 0-2% MeOH/DCM to afford 68 (43 mg, 47%) as white foam. ¹HNMR (DMSO-d6): δ 0.98-0.92 (m, 2H), 1.21-1.14 (m, 2H), 1.85-1.72 (m, 4H), 2.06-1.97 (m, 2H), 2.20-2.10 (m, 1H), 2.50-2.39 (m, 2H), 7.52-7.43 (m, 5H), 7.64 (dd, 2H), 7.78 (t, 1H), 7.90 (s, 1H), 8.05 (dt, 1H), 8.54 (dd, 1H), 8.63 (s, 1H), 8.87 (d, 1H). MS (ES⁺) m/z 436 (M+H)⁺.

### Example 69: Preparation of (E)-2-cyano-3-(3,5-dibromopyridin-2-yl)-N-(1-(4-(6-(trifluoromethyl) pyridin-3-yl)phenyl)cyclopentyl)acrylamide (Compound 68)

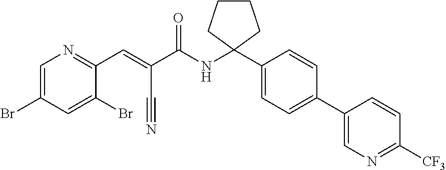 [SMILES: N#C/C(=C\c1ncc(Br)cc1Br)C(=O)NC1(c2ccc(-c3ccc(C(F)(F)F)nc3)cc2)CCCC1]

Following the procedure for Example 38, but using 2-(Trifluoromethyl)pyridine-5-boronic acid in place of (6-(Methoxycarbonyl)pyridin-3-yl)boronic acid, the title compound was prepared. MS (ES+) m/z 621 (M+H)⁺.

### Example 70: Preparation of (E)-2-cyano-3-(3,5-dibromopyridin-2-yl)-N-(1-(4-(6-(piperidin-1-yl) pyridin-3-yl)phenyl)cyclopentyl)acrylamide (Compound 69)

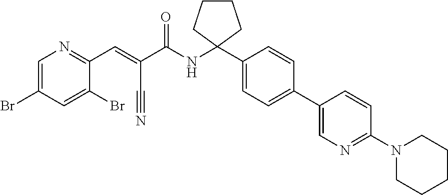 [SMILES: N#C/C(=C\c1ncc(Br)cc1Br)C(=O)NC1(c2ccc(-c3ccc(N4CCCCC4)nc3)cc2)CCCC1]

Following the procedure for Example 38, but using 6-(Piperidin-1-yl)pyridin-3-ylboronic acid in place of (6-(Methoxycarbonyl)pyridin-3-yl)boronic acid, the title compound was prepared. MS (ES+) m/z 636 (M+H)⁺.

### Example 71: Preparation of (E)-3-(6-(1H-indazol-5-yl)pyridin-2-yl)-2-cyano-N-(1-phenylcyclopentyl) acrylamide (Compound 70)

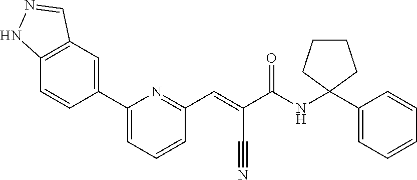 [SMILES: N#C/C(=C\c1cccc(-c2ccc3[nH]ncc3c2)n1)C(=O)NC1(c2ccccc2)CCCC1]

Following the procedure for Example 30, using 1H-Indazole-5-boronic acid in place of 3-pyridinylboronic acid, the title compound was prepared. MS (ES⁺) m/z 434 (M+H)⁺.

### Example 72: Preparation of (E)-3-(6-(2-cyano-3-oxo-3-((1-phenylcyclopentyl)amino)prop-1-en-1-yl)pyridin-2-yl)benzoic acid (Compound 71)

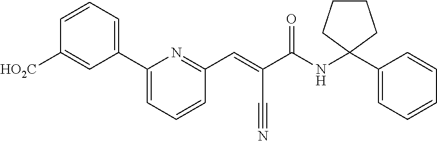 [SMILES: N#C/C(=C\c1cccc(-c2cccc(C(=O)O)c2)n1)C(=O)NC1(c2ccccc2)CCCC1]

Following the procedure for Example 30, using 3-Carboxybenzeneboronic acid in place of 3-pyridinylboronic acid, the title compound was prepared. MS (ES⁺) m/z 438 (M+H)⁺.

### Example 73: Preparation of (E)-3-(6-bromopyridin-2-yl)-2-cyano-N-(1-(4-((4-methoxybenzyl) oxy)phenyl)cyclopentyl)acrylamide (Compound 72)

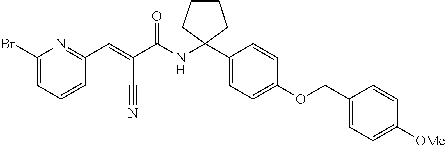 [SMILES: COc1ccc(COc2ccc(C3(NC(=O)/C(C#N)=C/c4cccc(Br)n4)CCCC3)cc2)cc1]

Following the procedure for Example 1, but using 1-{4-[(4-methoxyphenyl)methoxy]phenyl}cyclopentan-1-amine in place of 1-phenylcyclopentan-1-amine, the title compound was prepared. MS (ES+) m/z 532 (M+H)⁺.

### Example 74: Preparation of (E)-3-(6-bromopyridin-2-yl)-2-cyano-N-(1-(4-(pyridin-3-yl)phenyl) cyclopentyl)acrylamide (Compound 73)

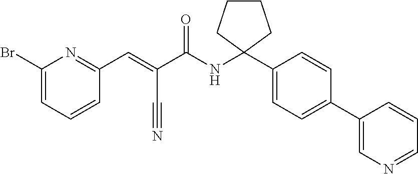 [SMILES: N#C/C(=C\c1cccc(Br)n1)C(=O)NC1(c2ccc(-c3cccnc3)cc2)CCCC1]

Following the procedure for Example 68, using 6-bromopyridine-2-carbaldehyde in place of Example 68 Intermediate (XI), the title compound was prepared. MS (ES⁺) m/z 434 (M+H)⁺.

### Example 75: Preparation of (E)-4′-(1-(3-(6-bromopyridin-2-yl)-2-cyanoacrylamido)cyclopentyl)-[1,1′-biphenyl]-3-carboxylic acid (Compound 74)

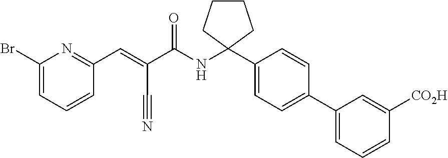 [SMILES: N#C/C(=C\c1cccc(Br)n1)C(=O)NC1(c2ccc(-c3cccc(C(=O)O)c3)cc2)CCCC1]

Following the procedure for Example 68, using 3-carboxyphenylboronic acid in place of pyridine-3-boronic acid and using 6-bromopyridine-2-carbaldehyde in place of Example 68 Intermediate (XI), the title compound was prepared. ¹HNMR (DMSO-d6): δ 1.90-1.74 (m, 4H), 2.09-1.96 (m, 2H), 2.50-2.42 (m, 2H), 7.65-7.49 (m, 5H), 7.77 (dd, 1H), 7.97-7.85 (m, 5H), 8.15 (s, 1H), 8.74 (s, 1H), 13.06 (s, 1H). MS (ES⁺) m/z 538 (M+Na)⁺.

### Example 76: Preparation of (E)-3-(6-bromopyridin-2-yl)-2-cyano-N-(1-(4-((3-hydroxyoxetan-3-yl) ethynyl)phenyl)cyclopentyl)acrylamide (Compound 75)

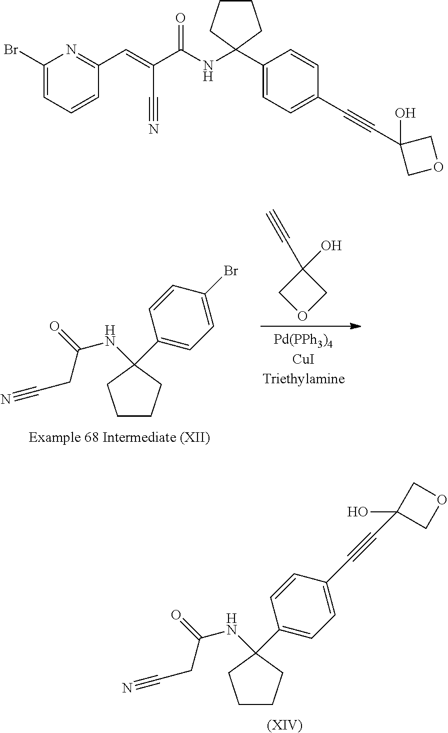

Step 1:

Following procedure for Example 45 Step 1, using Example 68 Intermediate (XII) in place of Example 32 Intermediate (I) and using 3-ethnyloxetan-3-ol in place of 1-(prop-2-yn-1-yl)-1H-pyrazole, Intermediate (XIV) was prepared. MS (ES⁺) m/z 325 (M+H)⁺.

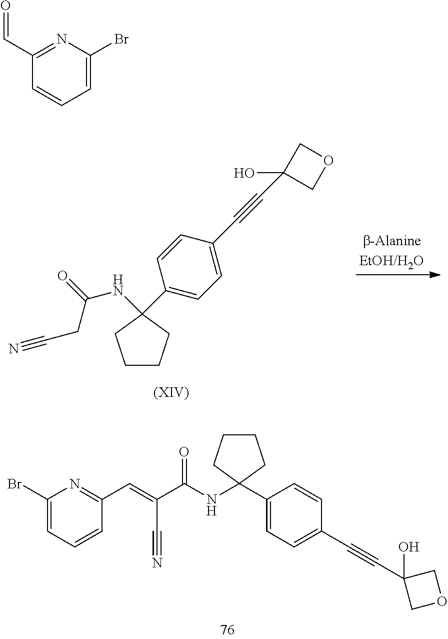

Step 2:

Following the procedure for Example 68 Step 4, using 6-bromopyridine-2-carbaldehyde in place of Example 68 Intermediate (XI) and using Intermediate (XIV) in place of Example 68 Intermediate (XIII), the title compound was prepared. ¹HNMR (DMSO-d6): δ 1.95-1.72 (m, 4H), 2.05-1.96 (m, 2H), 2.50-2.38 (m, 2H), 4.57 (d, 2H), 4.72 (d, 2H), 6.59 (s, 1H), 7.40-7.39 (m, 4H), 7.96-7.79 (m, 4H), 8.73 (s, 1H).

### Example 77: Preparation of (E)-3-(6-bromopyridin-2-yl)-2-cyano-N-(1-(4-((1-hydroxycyclopentyl) ethynyl)phenyl)cyclopentyl)acrylamide (Compound 76)

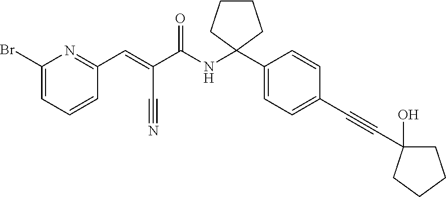 [SMILES: N#C/C(=C\c1cccc(Br)n1)C(=O)NC1(c2ccc(C#CC3(O)CCCC3)cc2)CCCC1]

Following the procedure for Example 76, 1-ethynylcyclopentanol in place of 3-ethnyloxetan-3-ol, the title compound was prepared. ¹HNMR (DMSO-d6): δ 1.97-1.70 (m, 14H), 2.40-2.36 (m, 2H), 5.28 (s, 1H), 7.38-7.28 (m, 4H), 7.78 (dd, 1H), 7.95-7.84 (m, 3H), 8.70 (s, 1H).

### Example 78: Preparation of (E)-2-cyano-3-(3,5-dibromopyridin-2-yl)-N-(1-(4-(1-methyl-1H-pyrazol-4-yl)phenyl)cyclopentyl)acrylamide (Compound 77)

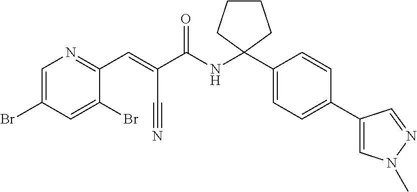 [SMILES: Cn1cc(-c2ccc(C3(NC(=O)/C(C#N)=C/c4ncc(Br)cc4Br)CCCC3)cc2)cn1]

Following the procedure for Example 68, using (1-methyl-1H-pyrazol-4-yl) boronic acid in place of pyridine-3-boronic acid and using 3,5-dibromopyridine-2-carbaldehyde in place of Example 68 Intermediate (XI), the title compound was prepared. MS (ES⁺) m/z 556 (M+H)⁺.

### Example 79: Preparation of (E)-3-(6-bromopyridin-2-yl)-2-cyano-N-(1-(4-(3-((2-hydroxyethyl) amino)prop-1-yn-1-yl)phenyl)cyclopentyl)acrylamide (Compound 78)

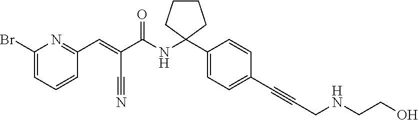 [SMILES: N#C/C(=C\c1cccc(Br)n1)C(=O)NC1(c2ccc(C#CCNCCO)cc2)CCCC1]

Following the procedure for Example 76, 2-(prop-2-yn-1-ylamino)ethan-1-ol hydrochloride in place of 3-ethnyloxetan-3-ol, the title compound was prepared. MS (ES⁺) m/z 494 (M+H)⁺.

### Example 80: Preparation of (E)-3-(6-bromopyridin-2-yl)-2-cyano-N-(1-(4-hydroxyphenyl) cyclopentyl)acrylamide (Compound 79)

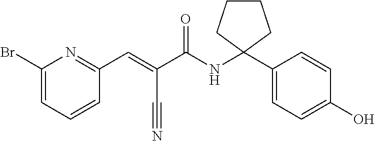 [SMILES: N#C/C(=C\c1cccc(Br)n1)C(=O)NC1(c2ccc(O)cc2)CCCC1]

Cyano acetic acid (1 eq) was dissolved in DCM at 0° C., and oxalyl chloride and (5 mL), cat. DMF was added and the mixture was stirred at RT. For 2 hours. The volatiles were evaporated and DCM, Et₃N (3 eq) and 1-{4-[(4-methoxyphenyl)methoxy]phenyl}cyclopentan-1-amine was added at 0° C. (4.5 g, 15.1 mmol), and stirred at RT for 4 h. 6-Bromopicolinaldehyde (4 eq), EtOH/H₂O, and β-alanine (16 eq) was added and stirred at 60° C., for 4 h. DDQ (3 eq) was added and the mixture was stirred in DCM at room temperature. The material was purified to give the final product. MS (ES+) m/z 412 (M+H)

### Example 81: Preparation of (E)-3-(6-bromopyridin-2-yl)-2-cyano-N-(3-phenyltetrahydrofuran-3-yl)acrylamide (Compound 80)

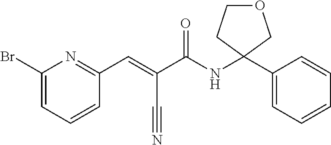 [SMILES: N#C/C(=C\c1cccc(Br)n1)C(=O)NC1(c2ccccc2)CCOC1]

Following the procedure for Example 1, but using 3-phenyloxolan-3-amine in place of 1-phenylcyclopentan-1-amine, the title compound was prepared. MS (ES+) m/z 398 (M+H)⁺.

### Example 82: Preparation of (E)-3-(6-bromopyridin-2-yl)-2-cyano-N-(1-(3-(hydroxymethyl)phenyl) cyclopentyl)acrylamide (Compound 81)

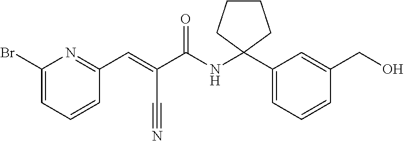 [SMILES: N#C/C(=C\c1cccc(Br)n1)C(=O)NC1(c2cccc(CO)c2)CCCC1]

Following the procedure for Example 1, but using [3-(1-aminocyclopentyl)phenyl]methanol in place of 1-phenylcyclopentan-1-amine, the title compound was prepared. MS (ES+) m/z 426 (M+H)⁺.

### Example 83: Preparation of (E)-4′-(1-(2-cyano-3-(6-cyclopropylpyridin-2-yl)acrylamido)cyclopentyl)-[1,1′-biphenyl]-3-carboxylic acid (Compound 82)

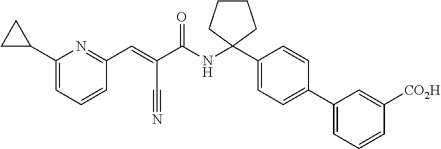 [SMILES: N#C/C(=C\c1cccc(C2CC2)n1)C(=O)NC1(c2ccc(-c3cccc(C(=O)O)c3)cc2)CCCC1]

Following the procedure for Example 68, using 3-carboxyphenylboronic acid in place of pyridine-3-boronic acid the title compound was prepared. MS (ES⁺) m/z 478 (M+H)⁺.

### Example 84: Preparation of (E)-2-cyano-N-(1-phenylcyclopentyl)-3-(6-(pyrrolidin-1-yl)pyridin-2-yl) acrylamide (Compound 83)

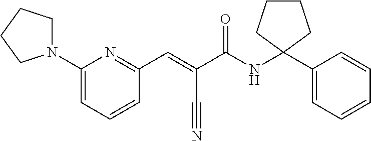 [SMILES: N#C/C(=C\c1cccc(N2CCCC2)n1)C(=O)NC1(c2ccccc2)CCCC1]

Following the procedure for Example 1, but using 6-Pyrrolidin-1-ylpyridine-2-carbaldehyde in place of 6-bromo-2-pyridinecarboxaldehyde, the title compound was prepared. MS (ES⁺) m/z 387 (M+H)⁺.

### Example 85: Preparation of (E)-4-(1-(3-(6-bromopyridin-2-yl)-2-cyanoacrylamido)cyclopentyl)benzyl tetradecanoate (Compound 84)

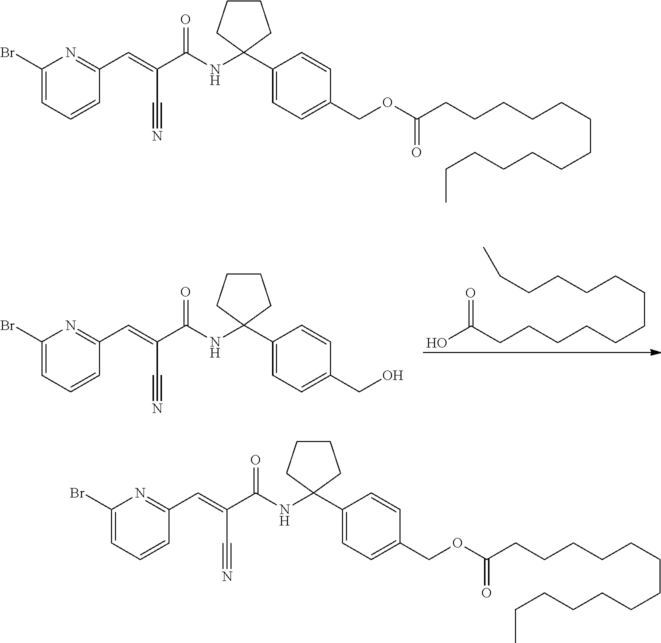 [SMILES: CCCCCCCCCCCCCC(=O)OCc1ccc(C2(NC(=O)/C(C#N)=C/c3cccc(Br)n3)CCCC2)cc1 ; N#C/C(=C\c1cccc(Br)n1)C(=O)NC1(c2ccc(CO)cc2)CCCC1 ; CCCCCCCCCCCCCC(=O)O ; CCCCCCCCCCCCCC(=O)OCc1ccc(C2(NC(=O)/C(C#N)=C/c3cccc(Br)n3)CCCC2)cc1]

In a 20 mL vial, a mixture of myristic acid (16 mg, 0.07 mmol), the starting benzyl alcohol (Compound 86) (30 mg, 0.07 mmol), EDCI (17 mg, 0.09 mmol), DMAP (0.8 mg, 0.007 mmol) was suspended in DCM (2 mL). The mixture was then stirred at room temperature for 2˜3 hours. After completion, the mixture was purified by silica gel chromatography 0-60% EtOAc/Hexane to afford 23 mg of title compound as a colorless oil. MS (ES⁺) m/z 658 (M+Na)⁺.

### Example 86: Preparation of (E)-4-(1-(3-(6-bromopyridin-2-yl)-2-cyanoacrylamido)cyclopentyl)benzyl 6-(2,5-dioxo-2,5-dihydro-1H-pyrrol-1-yl)hexanoate (Compound 85)

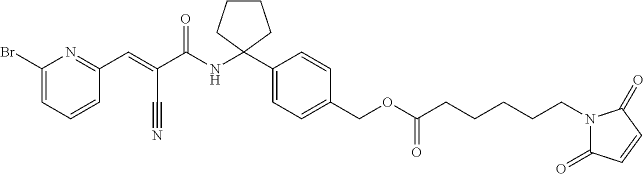 [SMILES: N#C/C(=C\c1cccc(Br)n1)C(=O)NC1(c2ccc(COC(=O)CCCCCN3C(=O)C=CC3=O)cc2)CCCC1]

Following the procedure for Example 85, using 6-Maleimidocaproic acid in place of myristic acid, the title compound was prepared. MS (ES⁺) m/z 641 (M+Na)⁺.

### Example 87: Preparation of (E)-3-(6-bromopyridin-2-yl)-2-cyano-N-(1-(4-(hydroxymethyl) phenyl)cyclopentyl)acrylamide (Compound 86)

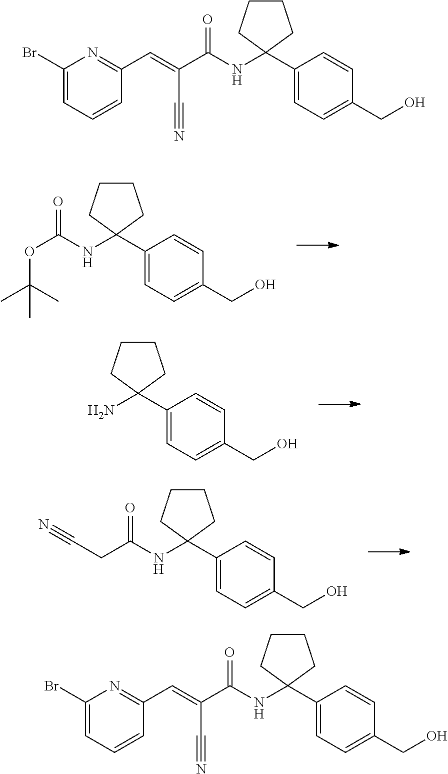

Step 1:

In a 20 mL vial, tert-butyl N-{1-[4-(hydroxymethyl)phenyl]cyclopentyl}carbamate (200 mg, 0.69 mmol) was suspended in 5 mL of 4 N HCl in dioxane. The mixture was then stirred at room temperature for overnight. The mixture was then evaporated to dryness. The product was titurated from cold ether to afford 100 mg of the desired amine.

Step 2:

Following the procedure for Example 1, but using amine from step 1 in place of 1-phenylcyclopentan-1-amine, the title compound was prepared. MS (ES⁺) m/z 448 (M+Na)⁺.

### Example 88: Preparation of (E)-4-(1-(3-(6-bromopyridin-2-yl)-2-cyanoacrylamido)cyclopentyl)benzyl acetate (Compound 87)

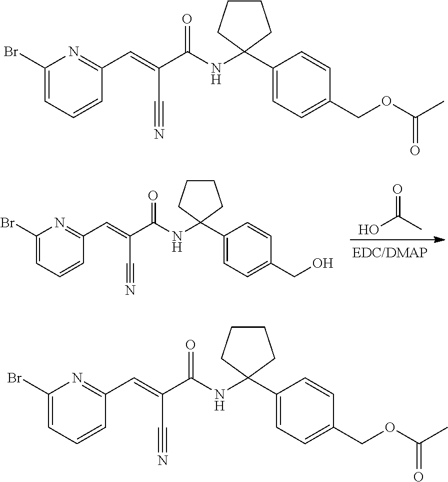

Following the procedure for Example 85, using acetic acid in place of myristic acid, the title compound was prepared. MS (ES⁺) m/z 490 (M+Na)⁺.

### Example 89: Preparation of (E)-tert-butyl 2-(4-(1-(3-(6-bromopyridin-2-yl)-2-cyanoacrylamido) cyclopentyl)phenoxy)acetate (Compound 88)

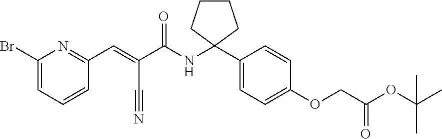 [SMILES: CC(C)(C)OC(=O)COc1ccc(C2(NC(=O)/C(C#N)=C/c3cccc(Br)n3)CCCC2)cc1]

Cyano aceticacid (1 eq) was dissolved in DCM at 0° C., and oxalyl chloride and (5 mL), cat. DMF was added and the mixture was stirred at RT. For 2 hours. The volatiles were evaporated and DCM, Et₃N (3 eq) and 1-{4-[(4-methoxyphenyl)methoxy]phenyl}cyclopentan-1-amine was added at 0° C. (4.5 g, 15.1 mmol), and stirred at RT for 4 h. 6-Bromopicolinaldehyde (4 eq), EtOH/H₂O, and β-alanine (16 eq) was added and stirred at 60° C., for 4 h. DDQ (3 eq0 was added and the mixture was stirred in DCM, RT. The material was purified and reacted with t-butylbromoacetate to give the final product after purification. MS (ES+) m/z 526 (M+H).

### Example 90: Preparation of (E)-3-(6-(2-cyano-3-oxo-3-((1-phenylcyclopentyl)amino)prop-1-en-1-yl) pyridin-2-yl)-2-fluorobenzoic acid (Compound 89)

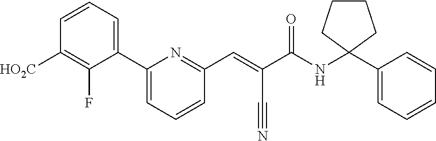 [SMILES: N#C/C(=C\c1cccc(-c2cccc(C(=O)O)c2F)n1)C(=O)NC1(c2ccccc2)CCCC1]

Following the procedure for Example 30, using 2-fluoro-3-(tetramethyl-1,3,2-dioxaborolan-2-yl)benzoic acid in place of 3-pyridinylboronic acid, the title compound was prepared. MS (ES⁺) m/z 456 (M+H)⁺.

### Example 91: Preparation of (E)-2-(4′-(1-(3-(6-bromopyridin-2-yl)-2-cyanoacrylamido)cyclopentyl)-[1,1′-biphenyl]-4-yl)acetic acid (Compound 90)

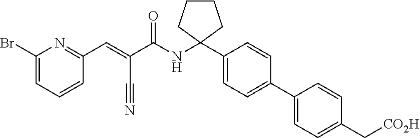 [SMILES: N#C/C(=C\c1cccc(Br)n1)C(=O)NC1(c2ccc(-c3ccc(CC(=O)O)cc3)cc2)CCCC1]

Following the procedure for Example 38, but using 6-bromo-2-pyridinecarboxaldehyde in place of 3,5-dibromopicolinaldehyde and using 2-[4-(tetramethyl-1,3,2-dioxaborolan-2-yl)phenyl]acetic acid in place of (6-(Methoxycarbonyl)pyridin-3-yl)boronic acid, the title compound was prepared. MS (ES+) m/z 552 (M+Na)⁺.

### Example 92: Preparation of (E)-4′-(1-(3-(6-bromopyridin-2-yl)-2-cyanoacrylamido)cyclopentyl)-[1,1′-biphenyl]-4-carboxylic acid (Compound 91)

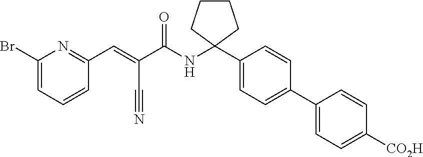 [SMILES: N#C/C(=C\c1cccc(Br)n1)C(=O)NC1(c2ccc(-c3ccc(C(=O)O)cc3)cc2)CCCC1]

Following the procedure for Example 38, but using 6-bromo-2-pyridinecarboxaldehyde in place of 3,5-dibromopicolinaldehyde and using 4-Carboxylphenylboronic acid pinacol ester in place of (6-(Methoxycarbonyl)pyridin-3-yl)boronic acid, the title compound was prepared. ¹H NMR (DMSO-d6): δ 1.75 (m, 4H), 1.96 (m, 2H), 2.38 (m, 2H), 7.50 (d, 2H), 7.66 (d, 2H), 7.78 (m, 3H), 7.85-8.01 (m, 5H), 8.75 (s, 1H), 12.95 (s, 1H)

### Example 93: Preparation of (E)-4′-(1-(2-cyano-3-(6-cyclopropylpyridin-2-yl)acrylamido)cyclopentyl)-[1,1′-biphenyl]-4-carboxylic acid (Compound 92)

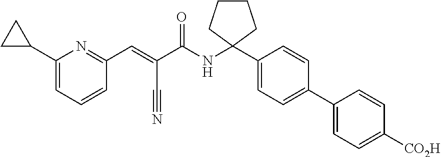 [SMILES: N#C/C(=C\c1cccc(C2CC2)n1)C(=O)NC1(c2ccc(-c3ccc(C(=O)O)cc3)cc2)CCCC1]

Following the procedure for Example 68, using 4-carboxyphenylboronic acid in place of pyridine-3-boronic acid the title compound was prepared. MS (ES⁺) m/z 478 (M+H)⁺.

### Example 94: Preparation of (E)-2-(4′-(1-(2-cyano-3-(6-cyclopropylpyridin-2-yl)acrylamido) cyclopentyl)-[1,1′-biphenyl]-4-yl)acetic acid (Compound 93)

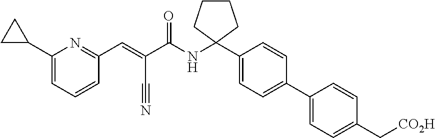 [SMILES: N#C/C(=C\c1cccc(C2CC2)n1)C(=O)NC1(c2ccc(-c3ccc(CC(=O)O)cc3)cc2)CCCC1]

Following the procedure for Example 68, using 4-(carboxymethyl)phenylboronic acid pinacol ester in place of pyridine-3-boronic acid the title compound was prepared. MS (ES⁺) m/z 492 (M+H)⁺.

### Example 95: Preparation of (E)-4′-(1-(3-(6-bromopyridin-2-yl)-2-cyanoacrylamido)cyclopentyl)-N-(phenylsulfonyl)-[1,1′-biphenyl]-3-carboxamide (Compound 94)

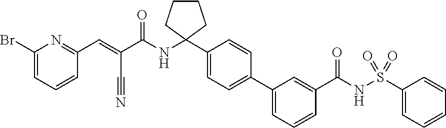 [SMILES: N#C/C(=C\c1cccc(Br)n1)C(=O)NC1(c2ccc(-c3cccc(C(=O)NS(=O)(=O)c4ccccc4)c3)cc2)CCCC1]

Example 75 (52 mg, 0.10 mmol), HATU (42 mg, 0.11 mmol), N,N-diisopropylethylamine (17.5 μL, 0.10 mmol), and DCM (2 mL) in 4 mL vial stirred at RT for 10 min. Benzenesulfonamide (17 mg, 0.11 mmol) and N,N-diisopropylethylamine (22.5 μL, 0.12 mmol) was added to the mixture and stirred at RT for 2 hrs. The reaction was washed with water, washed with brine, dried over Na₂SO₄, filtered, and concentrated under reduced pressure. The crude material was purified by silica gel chromatography 0-10% EtOAc/DCM to afford the title compound (39 mg, 59%) as an off white solid. MS (ES⁺) m/z 656 (M+H)⁺

### Example 96: Preparation of (E)-N-(1-(4-(1H-indazol-5-yl)phenyl)cyclopentyl)-3-(6-bromopyridin-2-yl)-2-cyanoacrylamide (Compound 95)

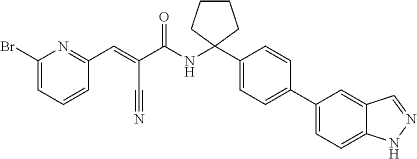 [SMILES: N#C/C(=C\c1cccc(Br)n1)C(=O)NC1(c2ccc(-c3ccc4[nH]ncc4c3)cc2)CCCC1]

Following the procedure for Example 38, but using 6-bromo-2-pyridinecarboxaldehyde in place of 3,5-dibromopicolinaldehyde and using 1H-Indazole-5-boronic acid in place of (6-(Methoxycarbonyl)pyridin-3-yl)boronic acid, the title compound was prepared. MS (ES+) m/z 534 (M+Na)⁺.

### Example 97: Preparation of (E)-N-(1-(4-(1H-indol-5-yl)phenyl)cyclopentyl)-3-(6-bromopyridin-2-yl)-2-cyanoacrylamide (Compound 96)

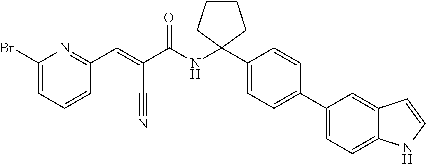 [SMILES: N#C/C(=C\c1cccc(Br)n1)C(=O)NC1(c2ccc(-c3ccc4[nH]ccc4c3)cc2)CCCC1]

Following the procedure for Example 38, but using 6-bromo-2-pyridinecarboxaldehyde in place of 3,5-dibromopicolinaldehyde and using 1H-Indole-5-boronic acid in place of (6-(Methoxycarbonyl)pyridin-3-yl)boronic acid, the title compound was prepared. MS (ES+) m/z 533 (M+Na)⁺.

### Example 98 Preparation of (E)-ethyl 2-(7-(6-(2-cyano-3-oxo-3-((1-phenylcyclopentyl)amino)prop-1-en-1-yl)pyridin-2-yl)-3,4-dihydroisoquinolin-2(1H)-yl)acetate (Compound 97)

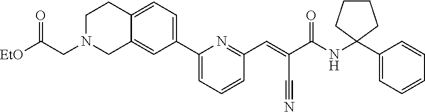 [SMILES: CCOC(=O)CN1CCc2ccc(-c3cccc(/C=C(\C#N)C(=O)NC4(c5ccccc5)CCCC4)n3)cc2C1]

Following the procedure for Example 30, using ethyl 2-[6-(tetramethyl-1,3,2-dioxaborolan-2-yl)-1,2,3,4-tetrahydroisoquinolin-2-yl]acetate in place of 3-pyridinylboronic acid, the title compound was prepared. MS (ES⁺) m/z 535 (M+H)⁺.

### Example 99: Preparation of (E)-2-(4′-(1-(3-(6-bromopyridin-2-yl)-2-cyanoacrylamido)cyclopentyl)-4-methyl-[1,1′-biphenyl]-3-yl)acetic acid (Compound 98)

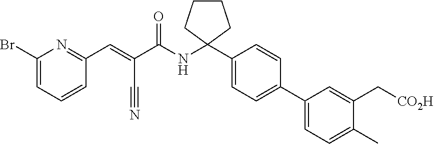 [SMILES: Cc1ccc(-c2ccc(C3(NC(=O)/C(C#N)=C/c4cccc(Br)n4)CCCC3)cc2)cc1CC(=O)O]

Following the procedure for Example 1, but using 2-{5-[4-(1-aminocyclopentyl)phenyl]-2-methylphenyl}acetic acid in place of 1-phenylcyclopentan-1-amine, the title compound was prepared. MS (ES+) m/z 544 (M+H).

### Example 100: Preparation of (E)-N-(1-(4-(1H-indol-5-yl)phenyl)cyclopentyl)-2-cyano-3-(6-cyclopropylpyridin-2-yl)acrylamide (Compound 99)

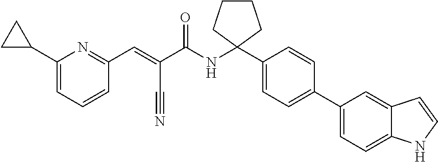 [SMILES: N#C/C(=C\c1cccc(C2CC2)n1)C(=O)NC1(c2ccc(-c3ccc4[nH]ccc4c3)cc2)CCCC1]

Following the procedure for Example 68, but using 1H-Indole-5-boronic acid in place of 2-Pyridineboronic acid, the title compound was prepared. MS (ES+) m/z 473 (M+H).

### Example 101: Preparation of (E)-3-(6-bromopyridin-2-yl)-2-cyano-N-(1-(pyridin-3-yl)cyclopentyl) acrylamide (Compound 3-1)

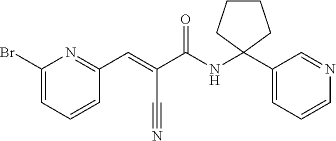 [SMILES: N#C/C(=C\c1cccc(Br)n1)C(=O)NC1(c2cccnc2)CCCC1]

Following the procedure of Example 1, but using 1-(pyridin-3-yl) cyclopentan-1-amine in place of 1-phenylcyclopentan-1-amine, title compound was prepared. MS (ES+) m/z 397 (M+H)⁺.

### Example 102: Preparation of (E)-3-(6-bromopyridin-2-yl)-2-cyano-N-(1-(pyridin-4-yl)cyclopentyl) acrylamide (Compound 3-2)

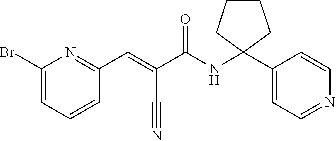 [SMILES: N#C/C(=C\c1cccc(Br)n1)C(=O)NC1(c2ccncc2)CCCC1]

Following the procedure for Example 1, but using 1-(pyridin-4-yl) cyclopentan-1-amine in place of 1-phenylcyclopentan-1-amine, the title compound was prepared. MS (ES+) m/z 397 (M+H)⁺.

### Example 103: Preparation of (E)-2-cyano-3-(3,5-dibromopyridin-2-yl)-N-(1-(pyridin-4-yl)cyclopentyl)acrylamide (Compound 3-3)

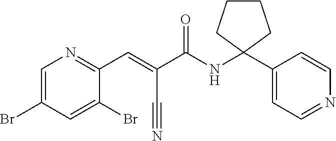 [SMILES: N#C/C(=C\c1ncc(Br)cc1Br)C(=O)NC1(c2ccncc2)CCCC1]

Following the procedure for Example 1, but using 3,5-dibromopicolinaldehyde in place of 6-bromo-2-pyridinecarboxaldehyde and using 1-(pyridin-4-yl) cyclopentan-1-amine in place of 1-phenylcyclopentan-1-amine, the title compound was prepared. MS (ES+) m/z 477 (M+H)⁺.

### Example 104: Preparation of (E)-3-(6-(3-(1H-pyrazol-1-yl)prop-1-yn-1-yl)pyridin-2-yl)-2-cyano-N-(1-(pyridin-4-yl)cyclopentyl)acrylamide (Compound 3-4)

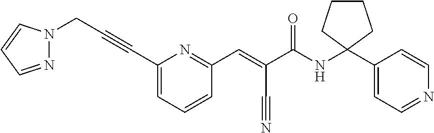 [SMILES: N#C/C(=C\c1cccc(C#CCn2cccn2)n1)C(=O)NC1(c2ccncc2)CCCC1]

Following the procedure for Example 44, but using 1-(pyridin-4-yl) cyclopentan-1-amine in place of 1-phenylcyclopentan-1-amine, the title compound was prepared. MS (ES+) m/z 423 (M+H)⁺.

### Example 105: Preparation of (E)-2-cyano-3-(64(1-hydroxycyclopentyl)ethynyl)pyridin-2-yl)-N-(1-(pyridin-4-yl)cyclopentyl)acrylamide (Compound 3-5)

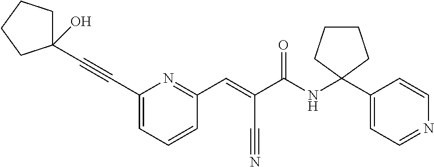 [SMILES: N#C/C(=C\c1cccc(C#CC2(O)CCCC2)n1)C(=O)NC1(c2ccncc2)CCCC1]

Following the procedure for Example 44, but using 1-ethynylcyclopentanol in place of 1-(prop-2-yn-1-yl)-1H-pyrazole and using 1-(pyridin-4-yl) cyclopentan-1-amine in place of 1-phenylcyclopentan-1-amine, the title compound was prepared. MS (ES+) m/z 427 (M+H)⁺.

### Example 106: Preparation of (E)-3-(6-bromopyridin-2-yl)-N-(1-(2-chloropyridin-4-yl)cyclopentyl)-2-cyanoacrylamide (Compound 3-6)

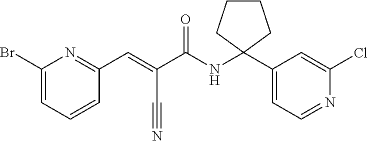 [SMILES: N#C/C(=C\c1cccc(Br)n1)C(=O)NC1(c2ccnc(Cl)c2)CCCC1]

Following the procedure for Example 1, but using 1-(2-chloro-4-yl) cyclopentan-1-amine in place of 1-phenylcyclopentan-1-amine, the title compound (Compound 3-7) was prepared. MS (ES+) m/z 431 (M+H)⁺.

### Example 107: Preparation of (E)-3-(6-bromopyridin-2-yl)-2-cyano-N-(1-(2-fluoropyridin-4-yl)cyclopentyl)acrylamide (Compound 3-7)

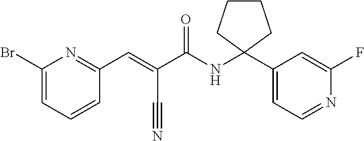 [SMILES: N#C/C(=C\c1cccc(Br)n1)C(=O)NC1(c2ccnc(F)c2)CCCC1]

Following the procedure for Example 1, but using 1-(2-fluoropyridin-4-yl) cyclopentan-1-amine in place of 1-phenylcyclopentan-1-amine, the title compound (Compound 3-7) was prepared. MS (ES+) m/z 415 (M+H)⁺.

### Biology Examples

### Example B-1: HA-Ubiqutin-Vinylsulfone (HA-Ub-VS) Cell Labeling

Inhibitition of DUB activity is measured in cells (NCI-H929, Z138, MM.1S, A375) using a hemaglutinin-ubiquitin-vinylsulfone (HA-Ub-VS) labeling assay. Briefly, 1-2×10⁶ cells are plated in 12-well plates in 1 ml complete growth media and 250 μl of test compound in 0.5% dimethylsulfoxide (or DMSO control) is added to each well. Plates are incubated at 37° C., 5% CO₂ for 2-6 hours before cells are harvested into eppendorf tubes, washed once in PBS and resuspended in 100 μl cold lysis buffer (50 mM Tris pH 8, 150 mM NaCl, 0.5% NP-40, 2 mM phenylmethylsulfonyl fluoride and complete protease inhibitor tablet). Resupended cells are frozen on dry ice, then thawed and incubated on ice for 15 minutes. Tubes are centrifuged for 10 minutes at 14,000 rpm at 4° C. and the cleared supernatants transferred to clean tubes. To assay for inhibition of DUB activity, 20-45 μl of the cleared supernatant is removed to a clean tube containing 2-5 μl of 2.5 μM HA-Ubiquitin-Vinylsulfone and mixed. The samples are incubated 15-60 min at 37° C. before the addition of SDS gel loading buffer. Proteins are separated by SDS-PAGE, transferred to nitrocellulose and the blot probed with anti-HA antibody and anti-β-actin antibodies. The HA and β-actin antibodies are detected using secondary antibodies coupled to IRdye 800 or IRdye 680 and visualized and quantitated using the LiCor Odyssey machine.

### Example B-2: Analysis of Md-1 Expression and PARP Cleavage

The ability of test compounds to decrease expression of the tumor promoting protein, Mcl-1, and to induce the inactivation/cleavage of the PARP protein is evaluated in cells (NCI-H929, Z138, MM.1S, A-375) by Western blotting. Cells are plated and treated as described in the HA-Ub-VS cell labeling assay (see above). A portion of the cleared lysate (15-50 μl) is run on an SDS-polyacrylamide gel to separate proteins and then transferred to nitrocellulose. After transfer to nitrocellulose, the proteins are detected by probing the blot with anti-Mcl-1 and anti-PARP antibodies followed by secondary antibodies coupled to IRdye 800 and IRdye 680 and visualization and quantitation using a LiCor Odyssey machine. Activity of the test compound is identified as a significant decrease in Mcl-1 expression relative to the vehicle treated cells and to the appearance of a cleaved fragment of PARP relative to the vehicle-treated cells.

The following compounds were tested in the above assays at 5 μM:

| | Labeling USP9x | PARP cleavage | Mcl-1 expression |
|---|---|---|---|
| Compound no. | % Inhibition | % Inhibition | % Inhibition |
| 1 | A | A | A |
| 2 | D | C | D |
| 3 | D | B | D |
| 4 | A | A | A |
| 5 | A | A | A |
| 6 | A | A | A |
| 7 | B | A | A |
| 8 | D | D | D |
| 9 | A | A | A |
| 10 | B | B | A |
| 11 | C | B | C |
| 12 | C | C | D |
| 13 | D | D | D |
| 14 | D | D | D |
| 15 | A | A | A |
| 16 | B | A | B |
| 17 | C | A | B |
| 18 | B | B | C |
| 19 | A | A | A |
| 20 | A | A | A |
| 21 | A | A | A |
| 22 | A | A | A |
| 23 | A | A | A |
| 24 | A | A | B |
| 25 | A | A | A |
| 26 | A | A | A |
| 27 | A | A | A |
| 28 | D | D | D |
| 29 | B | A | D |
| 30 | C | A | C |
| 31 | C | B | D |
| 32 | D | D | D |
| 33 | B | A | A |
| 34 | B | A | C |
| 35 | C | A | C |
| 36 | D | D | D |
| 37 | B | B | B |
| 38 | B | A | B |
| 39 | B | A | A |
| 40 | D | C | D |
| 41 | B | A | A |
| 42 | B | A | A |
| 43 | A | A | A |
| 44 | A | A | A |
| 45 | B | A | A |
| 46 | B | A | B |
| 47 | C | A | B |
| 48 | B | A | D |
| 49 | A | A | A |
| 50 | A | A | A |
| 51 | C | A | C |
| 52 | D | D | D |
| 53 | D | A | B |
| 54 | D | C | D |
| 55 | C | A | D |
| 56 | B | A | A |
| 57 | A | A | A |
| 58 | A | A | A |
| 59 | A | A | A |
| 60 | B | A | A |
| 61 | D | B | D |
| 62 | B | A | A |
| 63 | A | A | A |
| 64 | B | A | A |
| 65 | C | B | D |
| 66 | B | A | A |
| 67 | B | A | A |
| 68 | A | A | A |
| 69 | D | D | D |
| 70 | B | A | A |
| 71 | D | D | D |
| 72 | B | A | A |
| 73 | A | A | A |
| 74 | B | A | B |
| 75 | B | A | A |
| 76 | A | A | A |
| 77 | B | B | B |
| 78 | B | B | D |
| 79 | A | A | A |
| 80 | D | D | D |
| 81 | B | A | C |
| 82 | D | A | B |
| 83 | D | D | D |
| 84 | D | D | D |
| 85 | C | C | D |
| 86 | C | B | D |
| 87 | C | A | B |
| 88 | A | A | A |
| 89 | D | C | D |
| 90 | D | D | C |
| 91 | C | A | A |
| 92 | C | A | A |
| 93 | C | A | A |
| 94 | D | A | C |
| 95 | D | D | D |
| 96 | B | A | B |
| 97 | C | B | D |
| 98 | D | D | D |
| 99 | C | C | D |
| 2-82 | B | A | A |
| 3-1 | D | B | D |
| 3-2 | C | A | B |
| 3-3 | B | A | A |
| 3-4 | D | B | D |
| 3-5 | D | D | D |
| 3-6 | B | A | B |
| 3-7 | C | B | D |

A is ≧75% inhibition;
B is 50% to 75% inhibition;
C is 25% to 50% inhibition
D is <25% inhibition

### Example B-3: In Vivo Tumor Growth Assay

A xenograft model is used to test efficacy of compounds in inhibiting tumor growth. Briefly, 1×10⁶-2×10⁷ tumor cell suspensions (Z138, A375, MM.1S, VCaP) are injected subcutaneously into the flank regions of mice (SCID, NSG, nude) in an equal volume of matrigel. Tumor volumes are measured using calipers and when the tumor volumes are ˜100 mm³, test compound is administered every 1-3 days by oral gavage or by injection (intraperitoneal, intravenous, subcutaneous) until tumor volumes of the vehicle treated animals are ˜1000-2000 mm³. Inhibition of primary tumor growth is evident as a significant decrease in tumor volume relative to the vehicle treated mice.

Following the above protocol using SCID Beige mice injected with MM.1S myeloma tumor cells and dosing with Compound 74 at 15 mpk three times per week, an approximately 50% reduction in tumor volume was observed

The examples and embodiments described herein are for illustrative purposes only and various modifications or changes suggested to persons skilled in the art are to be included within the spirit and purview of this application and scope of the appended claims.

What is claimed is:

1. A compound that has the structure of Formula (I), or a pharmaceutically acceptable salt, or solvate thereof: 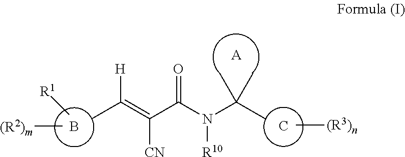
wherein,
ring A is a substituted or unsubstituted cycloalkylene, or substituted or unsubstituted heterocycloalkylene;
ring B is phenyl, naphthyl, or heteroaryl;
ring C is phenyl, naphthyl, or heteroaryl;
R¹ is selected from H, halogen, —OR⁴, —SR⁴, —N(R⁴)₂, —CN, substituted or unsubstituted alkyl, substituted or unsubstituted haloalkyl, substituted or unsubstituted phenyl, and substituted or unsubstituted heteroaryl;
each R² is independently selected from H, halogen, —OR⁴, —SR⁴, —N(R⁴)₂, —CN, —NO₂, substituted or unsubstituted alkyl, substituted or unsubstituted alkenyl, substituted or unsubstituted alkynyl, substituted or unsubstituted haloalkyl, —CO₂R⁴, —C(═O)N(R⁴)₂, —NHC(═O)R⁵, substituted or unsubstituted phenyl, and substituted or unsubstituted heteroaryl;
each R³ is independently selected from H, halogen, —CN, —NO₂, —OR⁴, —SR⁴, —S(═O)R⁵, —S(═O)₂R⁵, —S(═O)₂N(R⁴)₂, —NR⁴S(═O)₂R⁵, —C(═O)R⁵, —OC(═O)R⁵, —CO₂R⁴, —OCO₂R⁵, —N(R⁴)₂, —C(═O)N(R⁴)₂, —OC(═O)N(R⁴)₂, —NHC(═O)R⁵, —NHC(═O)OR⁵, unsubstituted or substituted alkyl, unsubstituted or substituted fluoroalkyl, unsubstituted or substituted alkenyl, unsubstituted or substituted alkynyl, unsubstituted or substituted heteroalkyl, unsubstituted or substituted cycloalkyl, unsubstituted or substituted aryl, unsubstituted or substituted heteroaryl, and -L³-L⁴-L⁵-L⁶-R⁹;
L³ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ᵣ—, or —(OCH₂CH₂)ᵣ—, r is 1, 2, 3, or 4;
L⁴ is absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, or —(OCH₂CH₂)ₛ—, s is 1, 2, 3, or 4;
L⁵ is absent, —O—, —S—, —S(O)—, —S(O)₂₋, —NR⁴—, —CH(OH)—, —C(═O)—, —C(═O)NH—, —NHC(═O)—, —C(═O)O—, —OC(═O)—, —OC(═O)NH—, —NHC(═O)NH—, —NHC(═O)O—, or —C(═O)NHS(O)₂—;
L⁶ is absent, unsubstituted or substituted alkylene, unsubstituted or substituted heteroalkylene;
R⁹ is H, halogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, or unsubstituted or substituted heteroaryl;
each R⁴ is independently selected from H, C₁-C₆alkyl, C₁-C₆fluoroalkyl, C₁-C₆deuteroalkyl, C₃-C₆cycloalkyl, a substituted or unsubstituted phenyl, or a substituted or unsubstituted monocyclic heteroaryl; or two R⁴ groups attached to the same N atom are taken together with the N atom to which they are attached to form a substituted or unsubstituted heterocycle;
R⁵ is C₁-C₆alkyl, C₁-C₆fluoroalkyl, C₁-C₆deuteroalkyl, C₃-C₆cycloalkyl, a substituted or unsubstituted phenyl, a substituted or unsubstituted monocyclic heteroaryl, or a substituted or unsubstituted bicyclic heteroaryl;
R¹⁰ is H, C₁-C₆alkyl, or C₁-C₆haloalkyl;
m is 0, 1, 2, or 3;
n is 0, 1, 2 or 3.

2. The compound, or pharmaceutically acceptable salt or solvate thereof, of claim 1, wherein:
ring A is a substituted or unsubstituted cycloalkylene that is a substituted or unsubstituted cyclopropylene, substituted or unsubstituted cyclobutylene, substituted or unsubstituted cyclopentylene, substituted or unsubstituted cyclohexylene, or substituted or unsubstituted cycloheptylene.

3. The compound, or pharmaceutically acceptable salt or solvate thereof, of claim 1 or claim 2, wherein the compound of Formula (I) has the structure of Formula (II): 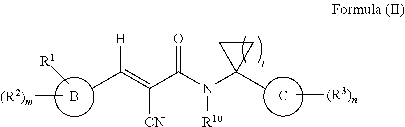
wherein,
t is 1, 2, 3, 4, or 5.

4. The compound, or pharmaceutically acceptable salt or solvate thereof, of claim 1, wherein:
ring A is a substituted or unsubstituted heterocycloalkylene that is a substituted or unsubstituted pyrrolidinyl, substituted or unsubstituted pyrrolidinonyl, substituted or unsubstituted tetrahydrofuranyl, substituted or unsubstituted tetrahydrofuranonyl, substituted or unsubstituted dihydrofuranonyl, substituted or unsubstituted dihydrofuranyl, substituted or unsubstituted tetrahydrothienyl, substituted or unsubstituted oxazolidinonyl, substituted or unsubstituted tetrahydropyranyl, substituted or unsubstituted dihydropyranyl, substituted or unsubstituted tetrahydrothiopyranyl, substituted or unsubstituted piperidinyl, substituted or unsubstituted morpholinyl, substituted or unsubstituted thiomorpholinyl, substituted or unsubstituted piperazinyl, substituted or unsubstituted aziridinyl, azetidinyl, substituted or unsubstituted oxetanyl, substituted or unsubstituted thietanyl, substituted or unsubstituted homopiperidinyl, substituted or unsubstituted oxepanyl, substituted or unsubstituted thiepanyl, substituted or unsubstituted oxazepinyl, substituted or unsubstituted diazepinyl, substituted or unsubstituted thiazepinyl, substituted or unsubstituted 1,2,3,6-tetrahydropyridinyl, substituted or unsubstituted indolinyl, or substituted or unsubstituted indolinonyl.

5. The compound, or pharmaceutically acceptable salt or solvate thereof, of claim 1, wherein the compound of Formula (I) has the structure of Formula (III): 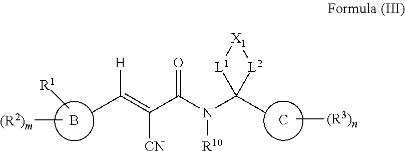
L¹ is —(C(R⁶)₂)ₚ—; p is 1, 2, 3, or 4;
L² is —(C(R⁶)₂)_{q}—; q is 0, 1, 2, 3, or 4;
X¹ is absent, —(C(R⁷)₂—, —O—, —S—, —S(O)—, —S(O)₂₋, —NR⁸—, —C(═O)—, —C(═O)NR⁴—, —C(═O)O—, —OC(═O)NR⁴—, or —NR⁴C(═O)NR⁴—;
each R⁶ is independently selected from H, halogen, —OR⁴, substituted or unsubstituted alkyl, substituted or unsubstituted haloalkyl, substituted or unsubstituted phenyl, and substituted or unsubstituted heteroaryl;
or two R⁶ on adjacent carbon atoms are taken together with the intervening atoms to form a monocyclic carbocycle or monocyclic heterocycle;
each R⁷ is independently selected from H, halogen, —OR⁴, substituted or unsubstituted alkyl, substituted or unsubstituted haloalkyl, substituted or unsubstituted phenyl, and substituted or unsubstituted heteroaryl;
or both R⁷ are taken together with the carbon atom to which they are attached to form a monocyclic carbocycle or monocyclic heterocycle;
each R⁸ is independently selected from H, —S(═O)R⁵, —S(═O)₂R⁵, —S(═O)₂N(R⁴)₂, —C(═O)R⁵, —CO₂R⁵, or —C(═O)N(R⁴)₂.

6. The compound, or pharmaceutically acceptable salt or solvate thereof, of claim 5, wherein:
X¹ is absent, —C(R⁷)₂—, —O—, —S—, —NR⁸—, or —C(═O)—.

7. The compound, or pharmaceutically acceptable salt or solvate thereof, of claim 5, wherein:
X¹ is absent.

8. The compound, or pharmaceutically acceptable salt or solvate thereof, of claim 5, wherein:
X¹ is —(C(R⁷)₂—;
each R⁷ is independently selected from H, halogen, —O—C₁-C₆alkyl, —O—C₁-C₆haloalkyl, C₁-C₆alkyl, or C₁-C₆haloalkyl;
or both R⁷ are taken together with the carbon atom to which they are attached to form a monocyclic carbocycle or monocyclic heterocycle.

9. The compound, or pharmaceutically acceptable salt or solvate thereof, of claim 5, wherein:
X¹ is —O— or —S.

10. The compound, or pharmaceutically acceptable salt or solvate thereof, of claim 5, wherein:
X¹ is —NR⁸—;
each R⁸ is independently selected from H, —S(═O)₂R⁵, or —C(═O)R⁵.

11. The compound, or pharmaceutically acceptable salt or solvate thereof, of any one of claims 5 to 10, wherein:
each R⁶ is H;
p is 1 or 2; and
q is 1 or 2.

12. The compound, or pharmaceutically acceptable salt or solvate thereof, of any one of claims 1 to 11, wherein:
ring B is phenyl.

13. The compound, or pharmaceutically acceptable salt or solvate thereof, of any one of claims 1 to 11, wherein:
ring B is monocyclic heteroaryl that is furanyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, or triazinyl.

14. The compound, or pharmaceutically acceptable salt or solvate thereof, of any one of claims 1 to 11, wherein:
ring B is pyridinyl, pyrimidinyl, pyrazinyl, pyridazinyl, or triazinyl.

15. The compound, or pharmaceutically acceptable salt or solvate thereof, of any one of claims 1 to 11, wherein:
ring B is pyridinyl.

16. The compound, or pharmaceutically acceptable salt or solvate thereof, of any one of claims 1 to 15, wherein:

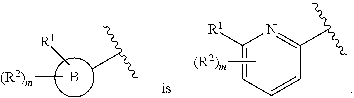

17. The compound, or pharmaceutically acceptable salt or solvate thereof, of any one of claims 1 to 16, wherein:
R¹ is selected from H, F, Cl, Br, —CN, substituted or unsubstituted alkyl, substituted or unsubstituted haloalkyl;
each R² is independently selected from H, halogen, —OR⁴, —SR⁴, —N(R⁴)₂, —CN, —NO₂, substituted or unsubstituted alkyl, substituted or unsubstituted haloalkyl.

18. The compound, or pharmaceutically acceptable salt or solvate thereof, of any one of claims 1 to 17, wherein:
R¹ is selected from H, F, Cl, or Br.

19. The compound, or pharmaceutically acceptable salt or solvate thereof, of any one of claims 1 to 18, wherein:
ring C is phenyl, naphthyl, monocyclic heteroaryl or bicyclic heteroaryl.

20. The compound, or pharmaceutically acceptable salt or solvate thereof, of any one of claims 1 to 19, wherein:
ring C is phenyl.

21. The compound, or pharmaceutically acceptable salt or solvate thereof, of any one of claims 1 to 19, wherein:
ring C is monocyclic heteroaryl that is furanyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, or triazinyl.

22. The compound, or pharmaceutically acceptable salt or solvate thereof, of any one of claim 1 to 19, or 21, wherein:
ring C is pyridinyl, pyrimidinyl, pyrazinyl, pyridazinyl, or triazinyl.

23. The compound, or pharmaceutically acceptable salt or solvate thereof, of any one of claims 1 to 19, wherein:
ring C is bicyclic heteroaryl that is quinolinyl, isoquinolinyl, quinazolinyl, quinoxalinyl, naphthyridinyl, indolyl, indazolyl, benzoxazolyl, benzisoxazolyl, benzofuranyl, benzothienyl, benzothiazolyl, benzimidazolyl, purinyl, cinnolinyl, phthalazinyl, pteridinyl, pyridopyrimidinyl, pyrazolopyrimidinyl, or azaindolyl.

24. The compound, or pharmaceutically acceptable salt or solvate thereof, of any one of claims 1 to 23, wherein:
each R³ is independently selected from H, halogen, —CN, —NO₂, —OR⁴, —SR⁴, —S(═O)R⁵, —S(═O)₂R⁵, —S(═O)₂N(R⁴)₂, —NR⁴S(═O)₂R⁵, —C(═O)R⁵, —OC(═O)R⁵, —CO₂R⁴, —OCO₂R⁵, —N(R⁴)₂, —C(═O)N(R⁴)₂, —OC(═O)N(R⁴)₂, —NHC(═O)R⁵, —NHC(═O)OR⁵, unsubstituted or substituted alkyl, unsubstituted or substituted fluroroalkyl, unsubstituted or substituted alkenyl, unsubstituted or substituted alkynyl, unsubstituted or substituted heteroalkyl, unsubstituted or substituted cycloalkyl, unsubstituted or substituted aryl, and unsubstituted or substituted heteroaryl.

25. The compound, or pharmaceutically acceptable salt or solvate thereof, of any one of claims 1 to 24, wherein:
each R³ is independently selected from H, halogen, —CN, —NO₂, —OR⁴, —SR⁴, —S(═O)R⁵, —S(═O)₂R⁵, —S(═O)₂N(R⁴)₂, —NR⁴S(═O)₂R⁵, —C(═O)R⁵, —OC(═O)R⁵, —CO₂R⁴, —OCO₂R⁵, —N(R⁴)₂, —C(═O)N(R⁴)₂, —OC(═O)N(R⁴)₂, —NHC(═O)R⁵, —NHC(═O)OR⁵, C₁-C₆alkyl and C₁-C₆fluoroalkyl.

26. The compound, or pharmaceutically acceptable salt or solvate thereof, of any one of claims 1 to 25, wherein:
each R³ is independently selected from H, halogen, —CN, —OR⁴, C₁-C₆alkyl and C₁-C₆fluroroalkyl.

27. The compound, or pharmaceutically acceptable salt or solvate thereof, of any one of claims 1 to 23, wherein:
R³ is -L³-L⁴-L⁵-L⁶-R⁹;
n is 1.

28. The compound, or pharmaceutically acceptable salt or solvate thereof, of claim 27, wherein:
-L³-L⁴-L⁵-L⁶- is C₁-C₆alkylene, —O—C₁-C₆alkylene, —NH—C₁-C₆alkylene, —(OCH₂CH₂)ᵣ—, —C₁-C₆alkylene-OC(═O)—C₁-C₆alkylene-, —C₁-C₆alkylene-NHC(═O)—C₁-C₆alkylene-, —C(═O)—C₁-C₆alkylene-C(═O)—, —(CH₂)ᵣ—OC(═O)—C₁-C₆alkylene-, —(CH₂)ᵣ—NHC(═O)—C₁-C₆alkylene-, —CH═N—NH—C(═O)—C₁-C₆alkylene-, or —C(CH₃)═N—NH—C(═O)—C₁-C₆alkylene-.

29. The compound, or pharmaceutically acceptable salt or solvate thereof, of claim 27 or claim 28, wherein:
R⁹ is unsubstituted or substituted monocyclic heterocycloalkyl or unsubstituted or substituted bicyclic heterocycloalkyl.

30. The compound, or pharmaceutically acceptable salt or solvate thereof, of any one of claims 27 to 29, wherein:
R⁹ is unsubstituted or substituted aziridinyl, unsubstituted or substituted azetidinyl, unsubstituted or substituted pyrrolidinyl, unsubstituted or substituted pyrrolidinonyl, unsubstituted or substituted oxazolidinonyl, unsubstituted or substituted piperidinyl, unsubstituted or substituted morpholinyl, unsubstituted or substituted thiomorpholinyl, unsubstituted or substituted piperazinyl, unsubstituted or substituted maleimidyl, or unsubstituted or substituted biotinyl.

31. The compound, or pharmaceutically acceptable salt or solvate thereof, of any one of claims 1 to 30, wherein:
R¹⁰ is H or C₁-C₆alkyl.

32. The compound, or pharmaceutically acceptable salt or solvate thereof, of any one of claims 1 to 31, wherein:
R¹⁰ is H.

33. The compound of claim 1, wherein the compound that has the structure of Formula (I) is:
(E)-3-(6-Bromopyridin-2-yl)-2-cyano-N-(1-phenylcyclopentyl)acrylamide;
(E)-3-(6-Bromopyridin-2-yl)-2-cyano-N-(1-phenylcyclopropyl)acrylamide;
(E)-3-(6-Bromopyridin-2-yl)-2-cyano-N-(1-phenylcyclobutyl)acrylamide;
(E)-3-(3,5-Dibromopyridin-2-yl)-2-cyano-N-(1-phenylcyclopentyl)acrylamide;
(E)-3-(3-Fluoro-5-chloropyridin-2-yl)-2-cyano-N-(1-phenylcyclopentyl)acrylamide;
(E)-3-(3-Fluoro-6-bromopyridin-2-yl)-2-cyano-N-(1-phenylcyclopentyl)acrylamide;
(E)-3-(3,6-Dichloropyridin-2-yl)-2-cyano-N-(1-phenylcyclopentyl)acrylamide;
(E)-3-(3-Fluoropyridin-2-yl)-2-cyano-N-(1-phenylcyclopentyl)acrylamide;
(E)-3-(5-Bromo-3-fluoropyridin-2-yl)-2-cyano-N-(1-phenylcyclopentyl)acrylamide;
(E)-3-(3-Bromo-6-chloropyridin-2-yl)-2-cyano-N-(1-phenylcyclopentyl)acrylamide;
(E)-3-(3,4-Dichlorophenyl)-2-cyano-N-(1-phenylcyclopentyl)acrylamide;
(E)-3-(4-Chloro-3-fluorophenyl)-2-cyano-N-(1-phenylcyclopentyl)acrylamide;
(E)-3-(4-Hydroxyphenyl)-2-cyano-N-(1-phenylcyclopentyl)acrylamide;
(E)-3-(3-Hydroxy-4-methoxyphenyl)-2-cyano-N-(1-phenylcyclopentyl)acrylamide;
(E)-N-(1-(4-bromophenyl)cyclopentyl)-3-(6-bromopyridin-2-yl)-2-cyanoacrylamide;
(E)-3-(6-Bromopyridin-2-yl)-2-cyano-N-(1-(4-methoxyphenyl)cyclopentyl)acrylamide;
(E)-3-(6-Bromopyridin-2-yl)-2-cyano-N-(1-phenylcyclohexyl)acrylamide;
(E)-3-(6-Bromopyridin-2-yl)-2-cyano-N-(4-phenyltetrahydro-2H-pyran-4-yl)acrylamide;
(E)-2-Cyano-3-(3,5-dibromopyridin-2-yl)-N-(1-(4-(trifluoromethyl)phenyl) cyclopentyl) acrylamide;
(E)-N-(1-(3-Chlorophenyl)cyclopentyl)-2-cyano-3-(3,5-dibromopyridin-2-yl)acrylamide;
(E)-2-Cyano-3-(3,5-dibromopyridin-2-yl)-N-(1-(4-fluorophenyl)cyclopentyl)acrylamide;
(E)-N-(1-(4-Chlorophenyl)cyclopentyl)-2-cyano-3-(3,5-dibromopyridin-2-yl)acrylamide;
(E)-2-Cyano-3-(3,5-dibromopyridin-2-yl)-N-(1-phenylcyclohexyl)acrylamide;
(E)-2-Cyano-3-(3,5-dibromopyridin-2-yl)-N-(4-phenyltetrahydro-2H-pyran-4-yl)acrylamide;
(E)-2-Cyano-3-(3,5-dibromopyridin-2-yl)-N-(1-phenylcycloheptyl)acrylamide;
(E)-2-cyano-3-(3,5-dibromopyridin-2-yl)-N-(1-(m-tolyl)cyclopentyl)acrylamide;
(E)-N-(1-(4-(tert-butyl)phenyl)cyclopentyl)-2-cyano-3-(3,5-dibromopyridin-2-yl)acrylamide;
(E)-3-(4-bromopyridin-2-yl)-2-cyano-N-(1-phenylcyclopentyl)acrylamide;
(E)-3-(5-bromopyridin-2-yl)-2-cyano-N-(1-phenylcyclopentyl)acrylamide;
(E)-3-([2,3′-bipyridin]-6-yl)-2-cyano-N-(1-phenylcyclopentyl)acrylamide;
(E)-ethyl 4-(6-(2-cyano-3-oxo-3-((1-phenylcyclopentyl)amino)prop-1-en-1-yl)pyridin-2-yl)benzoate;
(E)-ethyl 4′-(1-(2-cyano-3-(3,5-dibromopyridin-2-yl)acrylamido)cyclopentyl)-[1,1′-biphenyl]-4-carboxylate;
(E)-methyl 6-(2-cyano-3-oxo-3-((1-phenylcyclopentyl)amino)prop-1-en-1-yl)-[2,3′-bipyridine]-5′-carboxylate;
(E)-methyl 3-(6-(2-cyano-3-oxo-3-((1-phenylcyclopentyl)amino)prop-1-en-1-yl)pyridin-2-yl)benzoate;
(E)-2-cyano-3-(3,5-dibromopyridin-2-yl)-N-(1-phenylcyclopentyl)-N-(pyridin-3-ylmethyl)acrylamide;
(E)-2-(4-(6-(2-cyano-3-oxo-3-((1-phenylcyclopentyl)amino)prop-1-en-1-yl)pyridin-2-yl)phenoxy)acetic acid;
(E)-methyl 5-(4-(1-(2-cyano-3-(3,5-dibromopyridin-2-yl)acrylamido) cyclopentyl)phenyl)picolinate;
(E)-methyl 4′-(1-(2-cyano-3-(3,5-dibromopyridin-2-yl)acrylamido) cyclopentyl)-[1,1′-biphenyl]-3-carboxylate;
(E)-2-cyano-3-(3,5-dibromopyridin-2-yl)-N-(1-(4-(pyridin-3-yl)phenyl) cyclopentyl)acrylamide;
(E)-2-(4-(6-(2-cyano-3-oxo-3-((1-phenylcyclopentyl)amino)prop-1-en-1-yl)pyridin-2-yl)phenyl)acetic acid;
(E)-methyl 4-(1-(3-(6-bromopyridin-2-yl)-2-cyanoacrylamido) cyclopentyl)benzoate;
(E)-methyl 3-(6-((E)-2-cyano-3-oxo-3-((1-phenylcyclopentyl)amino)prop-1-en-1-yl)pyridin-2-yl)acrylate;
(E)-3-(6-(3-(1H-pyrazol-1-yl)prop-1-yn-1-yl)pyridin-2-yl)-2-cyano-N-(1-phenylcyclopentyl)acrylamide;
(E)-N-(1-(4-(3-(1H-pyrazol-1-yl)prop-1-yn-1-yl)phenyl)cyclopentyl)-2-cyano-3-(3,5-dibromopyridin-2-yl)-N-methylacrylamide;
(E)-2-cyano-3-(6-((1-hydroxycyclopentyl)ethynyl)pyridin-2-yl)-N-(1-phenylcyclopentyl)acrylamide;
(E)-3-(3-bromopyridin-2-yl)-2-cyano-N-(1-phenylcyclopentyl)acrylamide;
(E)-2-cyano-3-(2′-ethoxy-5′-(trifluoromethyl)-[2,3′-bipyridin]-6-yl)-N-(1-phenylcyclopentyl)acrylamide;
(E)-methyl 3-(1-(3-(6-bromopyridin-2-yl)-2-cyanoacrylamido) cyclopentyl)benzoate;
(E)-tert-butyl 6′-chloro-6-(2-cyano-3-oxo-3-((1-phenylcyclopentyl) amino)prop-1-en-1-yl)-[2,3′-bipyridine]-5′-carboxylate;
(E)-2-cyano-3-(3,5-dibromopyridin-2-yl)-N-(1-(4-(3-morpholinoprop-1-yn-1-yl)phenyl)cyclopentyl)acrylamide;
(E)-2-cyano-3-(3,5-dibromopyridin-2-yl)-N-(1-(4-(6-morpholinopyridin-3-yl)phenyl)cyclopentyl)acrylamide;
(E)-tert-butyl 3-(1-(2-cyano-3-(3,5-dibromopyridin-2-yl)acrylamido) cyclopentyl)benzoate;
(E)-2-cyano-3-(3,5-dibromopyridin-2-yl)-N-(1-(4-(2-methoxypyridin-3-yl)phenyl)cyclopentyl)acrylamide;
(E)-2-cyano-3-(3,5-dibromopyridin-2-yl)-N-(1-(4-(2-ethoxy-5-(trifluoromethyl)pyridin-3-yl)phenyl)cyclopentyl)acrylamide;
(E)-2-cyano-3-(6-(3-morpholinoprop-1-yn-1-yl)pyridin-2-yl)-N-(1-phenyl cyclopentyl)acrylamide;
(E)-tert-butyl 3-(6-(2-cyano-3-oxo-3-((1-phenylcyclopentyl)amino)prop-1-en-1-yl)pyridin-2-yl)benzoate;
(E)-2-cyano-3-(6′-morpholino-[2,3′-bipyridin]-6-yl)-N-(1-phenylcyclopentyl)acrylamide;
(E)-N-(1-(3-bromophenyl)cyclopentyl)-3-(6-bromopyridin-2-yl)-2-cyanoacrylamide;
(E)-N-(1-(4-(1H-pyrrolo[2,3-b]pyridin-5-yl)phenyl)cyclopentyl)-2-cyano-3-(3,5-dibromopyridin-2-yl)acrylamide;
(E)-ethyl 3-(6-(2-cyano-3-oxo-3-((1-phenylcyclopentyl)amino)prop-1-en-1-yl)pyridin-2-yl)-2-fluorobenzoate;
(E)-3-(6-(1H-pyrrolo[2,3-b]pyridin-5-yl)pyridin-2-yl)-2-cyano-N-(1-phenylcyclopentyl)acrylamide;
(E)-2-cyano-3-(6-(1-methyl-1H-pyrazol-4-yl)pyridin-2-yl)-N-(1-phenylcyclopentyl)acrylamide;
(E)-2-cyano-3-(3,5-dibromopyridin-2-yl)-N-(1-(4-(prop-1-en-2-yl) phenyl)cyclopentyl)acrylamide;
(E)-2-cyano-N-(1-phenylcyclopentyl)-3-(6′-(trifluoromethyl)-[2,3′-bipyridin]-6-yl)acrylamide;
(E)-2-cyano-N-(1-phenylcyclopentyl)-3-(6′-(piperidin-1-yl)-[2,3′-bipyridin]-6-yl)acrylamide;
(E)-4-(1-(2-cyano-3-(3,5-dibromopyridin-2-yl)acrylamido)cyclopentyl)-N,N-dimethylbenzamide;
(E)-2-cyano-3-(6-cyclopropylpyridin-2-yl)-N-(1-(4-(pyridin-3-yl)phenyl) cyclopentyl)acrylamide;
(E)-2-cyano-3-(3,5-dibromopyridin-2-yl)-N-(1-(4-(6-(trifluoromethyl) pyridin-3-yl)phenyl)cyclopentyl)acrylamide;
(E)-2-cyano-3-(3,5-dibromopyridin-2-yl)-N-(1-(4-(6-(piperidin-1-yl) pyridin-3-yl)phenyl)cyclopentyl)acrylamide;
(E)-3-(6-(1H-indazol-5-yl)pyridin-2-yl)-2-cyano-N-(1-phenylcyclopentyl) acrylamide;
(E)-3-(6-(2-cyano-3-oxo-3-((1-phenylcyclopentyl)amino)prop-1-en-1-yl)pyridin-2-yl)benzoic acid;
(E)-3-(6-bromopyridin-2-yl)-2-cyano-N-(1-(4-((4-methoxybenzyl) oxy)phenyl)cyclopentyl)acrylamide;
(E)-3-(6-bromopyridin-2-yl)-2-cyano-N-(1-(4-(pyridin-3-yl)phenyl) cyclopentyl)acrylamide;
(E)-4′-(1-(3-(6-bromopyridin-2-yl)-2-cyanoacrylamido)cyclopentyl)-[1,1′-biphenyl]-3-carboxylic acid;
(E)-3-(6-bromopyridin-2-yl)-2-cyano-N-(1-(4-((3-hydroxyoxetan-3-yl) ethynyl)phenyl)cyclopentyl)acrylamide;
(E)-3-(6-bromopyridin-2-yl)-2-cyano-N-(1-(4-((1-hydroxycyclopentyl) ethynyl)phenyl)cyclopentyl)acrylamide;
(E)-2-cyano-3-(3,5-dibromopyridin-2-yl)-N-(1-(4-(1-methyl-1H-pyrazol-4-yl)phenyl)cyclopentyl)acrylamide;
(E)-3-(6-bromopyridin-2-yl)-2-cyano-N-(1-(4-(3-((2-hydroxyethyl) amino)prop-1-yn-1-yl)phenyl)cyclopentyl)acrylamide;
(E)-3-(6-bromopyridin-2-yl)-2-cyano-N-(1-(4-hydroxyphenyl) cyclopentyl)acrylamide;
(E)-3-(6-bromopyridin-2-yl)-2-cyano-N-(3-phenyltetrahydrofuran-3-yl)acrylamide;
(E)-3-(6-bromopyridin-2-yl)-2-cyano-N-(1-(3-(hydroxymethyl)phenyl) cyclopentyl)acrylamide;
(E)-4′-(1-(2-cyano-3-(6-cyclopropylpyridin-2-yl)acrylamido)cyclopentyl)-[1,1′-biphenyl]-3-carboxylic acid;
(E)-2-cyano-N-(1-phenylcyclopentyl)-3-(6-(pyrrolidin-1-yl)pyridin-2-yl) acrylamide;
(E)-4-(1-(3-(6-bromopyridin-2-yl)-2-cyanoacrylamido)cyclopentyl)benzyl tetradecanoate;
(E)-4-(1-(3-(6-bromopyridin-2-yl)-2-cyanoacrylamido)cyclopentyl)benzyl 6-(2,5-dioxo-2,5-dihydro-1H-pyrrol-1-yl)hexanoate;
(E)-3-(6-bromopyridin-2-yl)-2-cyano-N-(1-(4-(hydroxymethyl) phenyl)cyclopentyl)acrylamide;
(E)-4-(1-(3-(6-bromopyridin-2-yl)-2-cyanoacrylamido)cyclopentyl)benzyl acetate;
(E)-tert-butyl 2-(4-(1-(3-(6-bromopyridin-2-yl)-2-cyanoacrylamido) cyclopentyl)phenoxy)acetate;
(E)-3-(6-(2-cyano-3-oxo-3-((1-phenylcyclopentyl)amino)prop-1-en-1-yl) pyridin-2-yl)-2-fluorobenzoic acid;
(E)-2-(4′-(1-(3-(6-bromopyridin-2-yl)-2-cyanoacrylamido)cyclopentyl)-[1,1′-biphenyl]-4-yl)acetic acid;
(E)-4′-(1-(3-(6-bromopyridin-2-yl)-2-cyanoacrylamido)cyclopentyl)-[1,1′-biphenyl]-4-carboxylic acid;
(E)-4′-(1-(2-cyano-3-(6-cyclopropylpyridin-2-yl)acrylamido)cyclopentyl)-[1,1′-biphenyl]-4-carboxylic acid;
(E)-2-(4′-(1-(2-cyano-3-(6-cyclopropylpyridin-2-yl)acrylamido) cyclopentyl)-[1,1′-biphenyl]-4-yl)acetic acid;
(E)-4′-(1-(3-(6-bromopyridin-2-yl)-2-cyanoacrylamido)cyclopentyl)-N-(phenylsulfonyl)-[1,1′-biphenyl]-3-carboxamide;
(E)-N-(1-(4-(1H-indazol-5-yl)phenyl)cyclopentyl)-3-(6-bromopyridin-2-yl)-2-cyanoacrylamide;
(E)-N-(1-(4-(1H-indol-5-yl)phenyl)cyclopentyl)-3-(6-bromopyridin-2-yl)-2-cyanoacrylamide;
(E)-ethyl 2-(7-(6-(2-cyano-3-oxo-3-((1-phenylcyclopentyl)amino)prop-1-en-1-yl)pyridin-2-yl)-3,4-dihydroisoquinolin-2(1H)-yl)acetate;
(E)-2-(4′-(1-(3-(6-bromopyridin-2-yl)-2-cyanoacrylamido)cyclopentyl)-4-methyl-[1,1′-biphenyl]-3-yl)acetic acid;
(E)-N-(1-(4-(1H-indol-5-yl)phenyl)cyclopentyl)-2-cyano-3-(6-cyclopropylpyridin-2-yl)acrylamide;
(E)-3-(6-bromopyridin-2-yl)-2-cyano-N-methyl-N-(1-phenylcyclopentyl)acrylamide;
(E)-3-(6-bromopyridin-2-yl)-2-cyano-N-(1-(pyridin-3-yl)cyclopentyl) acrylamide;
(E)-3-(6-bromopyridin-2-yl)-2-cyano-N-(1-(pyridin-4-yl)cyclopentyl) acrylamide;
(E)-2-cyano-3-(3,5-dibromopyridin-2-yl)-N-(1-(pyridin-4-yl)cyclopentyl)acrylamide;
(E)-3-(6-(3-(1H-pyrazol-1-yl)prop-1-yn-1-yl)pyridin-2-yl)-2-cyano-N-(1-(pyridin-4-yl)cyclopentyl)acrylamide;
(E)-2-cyano-3-(6-((1-hydroxycyclopentyl)ethynyl)pyridin-2-yl)-N-(1-(pyridin-4-yl)cyclopentyl)acrylamide;
(E)-3-(6-bromopyridin-2-yl)-N-(1-(2-chloropyridin-4-yl)cyclopentyl)-2-cyanoacrylamide;
(E)-3-(6-bromopyridin-2-yl)-2-cyano-N-(1-(2-fluoropyridin-4-yl)cyclopentyl)acrylamide;
or pharmaceutically acceptable salt or solvate thereof.

34. A pharmaceutical composition comprising a compound, or a pharmaceutically acceptable salt, or solvate thereof, of any one of claims 1 to 33, and at least one pharmaceutically acceptable excipient.

35. The pharmaceutical composition of claim 34, wherein the pharmaceutical composition is formulated for administration to a mammal by intravenous administration, subcutaneous administration, oral administration, inhalation, nasal administration, dermal administration, or ophthalmic administration.

36. The pharmaceutical composition of claim 34, wherein the pharmaceutical composition is in the form of a tablet, a pill, a capsule, a liquid, a suspension, a gel, a dispersion, a solution, an emulsion, an ointment, or a lotion.

37. A method of treating a disease or condition in a mammal that would benefit from the inhibition of the activity of at least one deubiquitinating enzyme comprising administering to the mammal a compound, or pharmaceutically acceptable salt or solvate thereof, of any one of claims 1 to 33.

38. The method of claim 37, wherein the disease or condition is cancer, fibrosis, an autoimmune disease or condition, an inflammatory disease or condition, a neurodegenerative disease or condition or an infection.

39. A method of treating cancer in a mammal comprising administering to the mammal a compound, or pharmaceutically acceptable salt or solvate thereof, of any one of claims 1 to 33.

40. The method of claim 39, wherein the cancer is a solid tumor.

41. The method of claim 39, wherein the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, or skin cancer.
